(19) United States
(12) Patent Application Publication
(10) Pub. No.: US 2024/0377415 A1
(43) Pub. Date: Nov. 14, 2024

(54) EXTRAMAMMARY PAGET DISEASE BIOMARKERS AND USES THEREOF

(71) Applicant: THE CATHOLIC UNIVERSITY OF KOREA INDUSTRY-ACADEMIC COOPERATION FOUNDATION, Seoul (KR)

(72) Inventor: Young-Bok LEE, Seoul (KR)

(21) Appl. No.: 18/658,276

(22) Filed: May 8, 2024

(30) Foreign Application Priority Data
May 8, 2023 (KR) 10-2023-0058921
May 3, 2024 (KR) 10-2024-0059150

(51) Int. Cl. G01N 33/68; A61K 45/06; G01N 33/50

(52) U.S. Cl. CPC G01N 33/6893; A61K 45/06; G01N 33/5023; G01N 2333/978; G01N 2800/365

(57) ABSTRACT

The present invention relates to extramammary Paget disease biomarkers, such as SPDEF, ARG2, and ABEP1, and uses thereof that were discovered by exploring the molecular profile as well as performing a comprehensive genetic analysis of EMPD. The present invention investigates how EMPD evolves in the context of treatment and may elucidate a potential mechanism of progression to invasion. Furthermore, the biomarkers of the present invention may be usefully used in the diagnosis or treatment of extramammary Paget disease in that the tissue microenvironments and cell types associated with EMPD may be further characterized using spatial transcriptomics.

### CROSS-REFERENCE TO RELATED APPLICATION

This application claims priority to Korean Patent Application No. 10-2023-0058921, filed on May 8, 2023, and Korean Patent Application No. 10-2024-0059150, filed on May 3, 2024, the disclosures of each of which are incorporated by reference herein in their entireties.

### BACKGROUND

### 1. Field of the Invention

The present invention relates to an extramammary Paget disease biomarker and a use thereof.

### 2. Discussion of Related Art

Extramammary Paget disease (hereinafter also referred to as “EMPD”) is a rare dermatological disease that generally develops in the skin containing apocrine glands, such as the vulva, perineum, scrotum, perianal area, and penile skin.

Treatment of EMPD is quite difficult due to the lack of established treatment guidelines, and it is also known that surgical management is difficult when the margins are unclear. Also, similar recurrence rates are noted among patients undergoing surgery with positive margins, which means that tumors are found at the edges of tumor areas that have been removed by local treatment or surgery.

Among topical agents, imiquimod stands out for its excellent response rate and improved recurrence-free survival results. Ingenol mebutate gel derived from Euphorbia peplus, also known as ground bug or milkweed, has shown potential clinical effectiveness in the treatment of EMPD. However, the lack of EMPD-dedicated clinical trials and the absence of specific treatments designed for its management pose distinct challenges.

As with many other diseases, the recent utilization of innovative technology based on next-generation sequencing has led to the discovery of new biomarkers and pathways associated with the pathology of EMPD. In one study, using single-cell RNA sequencing, the overexpression of MSII was detected in epithelial cells of EMPD, and mutations in genes involved in the RAS/RAF and PI3K/AKT pathways were also observed. Also, in EMPD, immune profiling using flow cytometry revealed an increase in exhausted CD8+ T cells.

Nevertheless, no markers for diagnosis or treatment of extramammary Paget disease have yet been reported.

### RELATED ART DOCUMENT

### Patent Document

Korean Patent Publication No. 10-2009-0005842

### SUMMARY OF THE INVENTION

Accordingly, it is an object of the present invention to provide a method of providing information for diagnosing or predicting the prognosis of extramammary Paget disease, which includes the following steps:

measuring the level of one or more selected from the group consisting of an SAM pointed domain containing ETS transcription factor (SPDEF), arginase 2 (ARG2), and adipocyte enhancer-binding protein 1 (ABEP1) from a biological sample isolated from a subject; and comparing the measured level of one or more selected from the group consisting of SPDEF, ARG2, and ABEP1 with the level in a biological sample isolated from the control.

It is another object of the present invention to provide a method of screening for an agent for preventing or treating extramammary Paget disease, which comprises the following steps:

measuring the level of any one or more selected from the group consisting of an SAM pointed domain containing ETS transcription factor (SPDEF), arginase 2 (ARG2), and adipocyte enhancer-binding protein 1 (ABEP1) from a biological sample isolated from an animal model of extramammary Paget disease administered a candidate material.

It is still another object of the present invention to provide a kit for diagnosing or predicting the prognosis of extramammary Paget disease, which comprises the following:
i) a composition including, as an active ingredient, an agent for measuring the level of one or more selected from the group consisting of an SAM pointed domain containing ETS transcription factor (SPDEF), arginase 2 (ARG2), and adipocyte enhancer-binding protein 1 (ABEP1); and
ii) instructions.

It is yet another object of the present invention to provide a kit for screening for an agent for preventing or treating extramammary Paget disease, which comprises the following:
i) a composition including, as an active ingredient, an agent for measuring the level of one or more selected from the group consisting of an SAM pointed domain containing ETS transcription factor (S PDEF), arginase 2 (ARG2), and adipocyte enhancer-binding protein 1 (ABEP1); and
ii) instructions.

However, the technical objects to be achieved by the present invention are not limited to the above-described technical objects, and other objects which are not mentioned above will be clearly understood from the following detailed description by those skilled in the art to which the present invention pertains.

According to an aspect of the present invention, there is provided a method of providing information for diagnosing or predicting the prognosis of extramammary Paget disease, which comprises the following steps:

measuring the level of one or more selected from the group consisting of an SAM pointed domain containing ETS transcription factor (SPDEF), arginase 2 (ARG2), and adipocyte enhancer-binding protein 1 (ABEP1) from a biological sample isolated from a subject; and

comparing the measured level of one or more selected from the group consisting of SPDEF, ARG2, and ABEP1 with the level in a biological sample isolated from the control.

According to one embodiment of the present invention, the method of providing information may further comprise:

when the level of any one or more selected from the group consisting of SPDEF, ARG2, and ABEP1 in the biological sample isolated from the subject is higher than the level in the biological sample isolated from the control, determining this case to be extramammary Paget disease, but the present invention is not limited thereto.

According to one embodiment of the present invention, wherein the extramammary Paget's disease is non-invasive extramammary Paget's disease, or invasive extramammary Paget's disease, but the present invention is not limited thereto.

According to one embodiment of the present invention, wherein the non-invasive extramammary Paget's disease is early extramammary Paget's disease, or non-early extramammary Paget's disease, but the present invention is not limited thereto.

According to one embodiment of the present invention, the method of providing information may further comprise:

when the level of ARG2 in the biological sample isolated from the subject is higher than the level in the biological sample isolated from the control, determining this case to be non-invasive extramammary Paget disease; or

when the level of ABEP1 in the biological sample isolated from the subject is higher than the level in the biological sample isolated from the control, determining this case to be invasive extramammary Paget disease, but the present invention is not limited thereto.

According to one embodiment of the present invention, When measuring the level of ARG2, the control is a normal control group; or

When measuring the level of ABEP1, the control is a normal control group, or an individual with non-invasive extramammary Paget's disease, but the present invention is not limited thereto.

According to one embodiment of the present invention, the biological sample may be any one selected from the group consisting of tissue, blood, serum, whole blood, plasma, urine, saliva, cells, organs, bone marrow, a fine needle aspiration specimen, a core needle biopsy specimen, and a vacuum-assisted suction biopsy specimen, but the present invention is not limited thereto.

According to one embodiment of the present invention, the method of providing information may further comprise:

When the biological sample isolated from the subject is a pre-Paget cell,

when the level of any one or more selected from the group consisting of SPDEF and AGR2 in the biological sample isolated from the subject is higher than the level in the biological sample isolated from the control, determining this case to early extramammary Paget's disease, but the present invention is not limited thereto.

According to one embodiment of the present invention, the invasive extramammary Paget disease may be extramammary Paget disease that persists despite the administration of an extramammary Paget disease drug, but the present invention is not limited thereto.

According to another aspect of the present invention, there is provided a method of screening for an agent for preventing or treating extramammary Paget disease, which comprises the following steps:

measuring the level of any one or more selected from the group consisting of an SAM pointed domain containing ETS transcription factor (SPDEF), arginase 2 (ARG2), and adipocyte enhancer-binding protein 1 (ABEP1) from a biological sample isolated from an animal model of extramammary Paget disease administered a candidate material; and

when the level of any one or more selected from the group consisting of SPDEF, ARG2, and AEBP1 in the biological sample isolated from the animal model decreases, selecting the candidate material as an agent for preventing or treating extramammary Paget disease.

According to one embodiment of the present invention, the screening method may further comprise:

when the level of AGR2 in the biological sample isolated from the animal model decreases, selecting the candidate material as an agent for preventing or treating non-invasive extramammary Paget disease; or

when the level of ABEP1 in the biological sample isolated from the animal model decreases, selecting the candidate material as an agent for preventing or treating invasive extramammary Paget disease, but the present invention is not limited thereto.

According to still another aspect of the present invention, there is provided a kit for diagnosing or predicting the prognosis of extramammary Paget disease, which comprises the following:
i) a composition comprising, as an active ingredient, an agent for measuring the level of one or more selected from the group consisting of an SAM pointed domain containing ETS transcription factor (SPDEF), arginase 2 (ARG2), and adipocyte enhancer-binding protein 1 (ABEP1); and
ii) instructions.

According to yet another aspect of the present invention, there is provided a kit for screening for an agent for preventing or treating extramammary Paget disease, which comprises the following:
i) a composition comprising, as an active ingredient, an agent for measuring the level of one or more selected from the group consisting of an SAM pointed domain containing ETS transcription factor (SPDEF), arginase 2 (ARG2), and adipocyte enhancer-binding protein 1 (ABEP1); and
ii) instructions.

According to yet another aspect of the present invention, there is provided a method of preventing or treating extramammary Paget disease, which comprises the following steps:

measuring the level of one or more selected from the group consisting of an SAM pointed domain containing ETS transcription factor (SPDEF), arginase 2 (ARG2), and adipocyte enhancer-binding protein 1 (ABEP1) from a biological sample isolated from a subject;

comparing the measured level of one or more selected from the group consisting of SPDEF, ARG2, and ABEP1 with the level in a biological sample isolated from the control;

when the level of any one or more selected from the group consisting of SPDEF, ARG2, and ABEP1 in the biological sample isolated from the subject is higher than the level in the biological sample isolated from the control, determining this case to be non-invasive extramammary Paget disease; and

administering an agent for preventing or treating extramammary Paget disease to a subject determined to have extramammary Paget disease.

According to yet another aspect of the present invention, there is provided a marker for treating non-invasive extramammary Paget disease or invasive extramammary Paget disease, which comprises any one or more selected from the group consisting of an SAM pointed domain containing ETS transcription factor (SPDEF), arginase 2 (ARG2), and adipocyte enhancer-binding protein 1 (ABEP1).

In addition, the present invention provides a marker for treating extramammary Paget's disease, comprising at least one selected from the group consisting of SPDEF, ARG2, and ABEP1.

Additionally, the present invention provides a marker for treating extramammary Paget's disease, comprising SPDEF. At this time, extramammary Paget's disease is non-invasive or invasive extramammary Paget's disease.

Additionally, the present invention provides a marker for treating non-invasive extramammary Paget's disease, comprising ARG2.

Additionally, the present invention provides a marker for treating invasive extramammary Paget's disease, comprising SPDEF, and ABEP1.

Additionally, the present invention provides a marker for preventing extramammary Paget's disease, comprising SPDEF, and ARG2.

According to yet another aspect of the present invention, there is provided a composition for diagnosing or predicting prognosis of extramammary Paget disease, non-invasive extramammary Paget disease or invasive extramammary Paget disease, which comprises, as an active ingredient, an agent for measuring the level of any one or more selected from the group consisting of an SAM pointed domain containing ETS transcription factor (SPDEF), arginase 2 (ARG2), and adipocyte enhancer-binding protein 1 (ABEP1).

According to yet another aspect of the present invention, there is provided a composition for screening for a material for preventing or treating extramammary Paget disease, in particular, non-invasive extramammary Paget disease or invasive extramammary Paget disease, which comprises, as an active ingredient, an agent for measuring the level of any one or more selected from the group consisting of an SAM pointed domain containing ETS transcription factor (SPDEF), arginase 2 (ARG2), and adipocyte enhancer-binding protein 1 (ABEP1).

The present invention provides a use for diagnosing or predicting prognosis of extramammary Paget's disease using a composition comprising as an active ingredient an agent that measures the level of one or more selected from the group consisting of SPDEF, ARG2, and ABEP1.

The present invention provides the use of a composition comprising as an active ingredient an agent that measures the level of one or more selected from the group consisting of SPDEF, ARG2, and ABEP1 for preparing an agent for diagnosing or predicting prognosis of extramammary Paget's disease.

### BRIEF DESCRIPTION OF THE DRAWINGS

FIGS. 1A to 1H show the digital spatial profiling of normal, EMPD, and invasive EMPD.

FIG. 1A is a schematic diagram of a graphical description of a method used in this study.

FIG. 1B is a representative H&E staining and immunofluorescence (IF) image of EMPD tissue. Here, the selected area is indicated by a polygonal shape (Bar=300 μm).

FIG. 1C is an alluvial diagram showing the sample analysis throughout the study. In the diagram, the width of the connecting lines corresponds to the number of samples at each stage.

FIG. 1D is a 2D plot showing the distribution of treatment patterns over time for three different groups and a correlation plot to evaluate relationships between samples. Disease groups are coded in three different colors, with symbols representing samples corresponding to different treatment time points. The sidebar legend indicates the color of each sample. Each data point in the figure corresponds to a pair of samples, and the color intensity indicates the degree to which the characteristics of the samples are similar or different. The box highlighted in red indicates the area showing the most positive correlation with normal epidermis.

FIG. 1E is a comprehensive table of analyzed regions of interest (ROIs), presenting histological classification and various pathological parameters.

FIG. 1F is a graphical representation showing the correlation of FIG. 1D in numbers.

FIG. 1G shows a dot plot displaying the ssGSEA score of each sample using known Paget Cell Markers 1 and 2.

FIGS. 2A to 2E show the temporal dynamics of histone-related gene expression and pathway activity in EMPD after treatment.

FIG. 2A is a PCA polygon plot examining changes in EMPD during various time periods after treatment. Here, colors corresponding to the duration are indicated in the legend bar.

FIG. 2B is a volcano plot created to identify DEGs between samples at baseline EMPD and one year after treatment. Here, both green and gray dots show non-significant results. The green dots meet the log₂ fold change criteria, but do not meet the FDR, while the gray dots do not meet both criteria.

FIG. 2C is a Venn diagram created to examine genes having significant differences in both log₂ fold change and FDR when compared to normal samples at four time points. For color coding for each time point, refer to the legend provided next to this diagram.

FIG. 2D shows the top 20 major pathways based on DEGs identified at a 4-month time point compared to baseline EMPD.

FIG. 2E is a heatmap showing the expression levels of histone genes and genes that regulate histones in the G1 to S cell cycle of EMPD at various time points (PCA: principal component).

FIGS. 2F to 2I show the immune cell profiles of EMPD at different time points.

FIG. 2F shows the relative fraction of immune cells in each sample using CIBERSORTx. Color coding for different cell types is shown in the row legend bar, and group classification is shown in the column legend bar.

FIG. 2G shows the distribution of lymphoid and myeloid cells depicted in dot plots.

FIG. 2H shows bar plots (upper graphs) of the expression of PD-1 (PDCD1) and PD-L1 (CD274) genes, and dot plots (lower graphs) of markers associated with exhausted CD8+ T cells.

FIG. 2I shows genes associated with MHC1. Here, annotations for different time periods are indicated in the legend bars.

FIGS. 3A to 3I shows the results of confirming that EMPD includes an mTOR pathway and is regulated by an SPDEF gene.

FIG. 3A is a volcano plot comparing baseline EMPD to normal and invasive samples. Genes marked with red dots are up-regulated, while genes marked with blue dots are down-regulated. Gray genes do not meet the criteria for the log₂ fold change and FDR value. Labeled genes represent those of particular note in each sample.

FIGS. 3B and 3C show the GO analysis results for baseline EMPD vs. normal samples and invasive samples vs. baseline EMPD. The size of the dots represents the counts associated with each process, and the color represents the p-value.

FIG. 3D is a graph comparing overall EMPD with the normal group. Here, genes up-regulated in overall EMPD were used as inputs to Metascape. From the top, bar groups of enrichment of up-regulated genes colored by p-value, bar graphs of enrichment analysis of DisGeNet and PaGenBase, and bar graphs of enrichment analysis of TRRUST are shown.

FIG. 3E shows a significant mTORC1 pathway and top 10 leading-edge genes in baseline EMPD and invasive EMPD.

FIG. 3F shows the results of mTORC1 pathway-related analysis. The top diagram shows the results of heatmap analysis of mTORC1-related downstream gene expression in normal, EMPD, and invasive samples. The bottom diagram shows the results of heatmap analysis of EMT genes related to the mTORC1 pathway and myc gene association in invasive EMPD.

FIG. 3G shows the results of comparative analysis of gene expression affected by HIF1A.

FIG. 3H shows invasive EMPD showing high normalized enrichment score (NES) values, and shows the results observed in invasive EMPD samples showing a significant decrease in the activity of myc and p53 pathways.

FIG. 3I is a correlation graph showing the association between SPDEF and known Paget cell markers. The x-axis represents SPDEF and the y-axis represents known Paget cell markers. P-values and correlation coefficient values (Cor) are shown in the upper left corner of each graph (NES: normalized enrichment score; FDR: false discovery rate).

FIG. 3J shows a list of AEBP1 regulatory transcription factors in invasive EMPD in order from the lowest value to highest value. The heatmap shows the lack of consistent patterns in EMPD by visualizing target genes regulated by AEBP1. Column colors represent groups as detailed in the bar legend.

FIG. 3k shows the results of heatmap analysis to confirm the relationship between SPDEF, and baseline EMPD and invasive EMPD. Expression of genes related to SPDEF was confirmed at a high level in both EMPD and invasive EMPD, and SPDEF was identified as EMPD, And it indicates that it is a major marker of invasive EMPD.

FIG. 4A shows the results of UMAP visualization analysis of EMPD cell types and perilesional skin samples.

FIG. 4B shows a bar graph of cell type ratios shown in FIG. 4A.

FIG. 4C shows a dot plot of genetic markers.

FIG. 4D shows a volcano plot of differential gene expression between Paget cells and pre-Paget cells.

FIG. 4E shows the UMAP of AQP5 (pre-Paget marker) and EIF4EBP1 (Paget marker) co-expression patterns.

FIGS. 4F and 4G show the cell types of immune subpopulations and genetic markers of immune subpopulation cell types, respectively.

FIGS. 4H and 4I show the subclusters for fibroblasts and genetic markers for fibroblast subtypes, respectively.

FIG. 5A shows the InferCNV heatmap of cell types divided by sample source.

FIG. 5B shows the copy number variations displayed in UMAP.

FIG. 5C shows the copy number variation scores displayed as violin plots by cell type and sample source.

FIG. 6A shows a random walk plot.

FIG. 6B shows the UMAP of a PAGA speed graph.

FIG. 6C shows a circular projection plot according to the fate probability for pre-Paget cells leading to Paget cells.

FIG. 6D shows a heatmap of fate probabilities.

FIG. 7A shows the top 20 lineage driver genes in Paget cells.

FIG. 7B shows lineage driver gene expression according to latent time.

FIG. 8A shows important differential signaling pathways between sample conditions.

FIG. 8B shows the overall signal patterns regarding the state.

FIG. 8C shows integration2 with public EMPD data.

FIG. 8D shows the analysis of the top 15 influential receptor-ligands of the integrated EMPD atlas for EMPD cell types.

FIG. 9A shows canonical pathways within cell types.

FIG. 9B shows the prediction of potential drug efficacy.

### DETAILED DESCRIPTION OF EXEMPLARY EMBODIMENTS

The present inventors discovered for the time that several pathways, including an ROS pathway and an EMT pathway, are characteristically observed in EMPD subjects, and that the tendency of these pathways increases as invasive EMPD progresses. In particular, the mTOR pathway was confirmed to be an important mechanism in both non-invasive and invasive EMPD.

It was confirmed that SPDEF in a downstream stage of the mTOR pathway is a significant marker observed throughout the disease after the diagnosis of EMPD because a gene transcript in which SPDEF is significantly up-regulated includes non-invasive EMPD, invasive EMPD, and even early-stage EMPD that can be identified by pre-Paget cells.

Meanwhile, as described above, as non-invasive EMPD progresses into invasive EMPD, genes related to ROS and EMT pathways are up-regulated, and in particular, HIF1α appears to increase significantly. In addition, among the genes affected by HIF1α, AEBP1 was confirmed to show a distinct up-expression pattern in invasive EMPD.

In addition, the present inventors discovered pre-Paget cells as potential lesional tissues destined to differentiate into Paget cells.

In the present invention, SPDEF and AGR2 were identified in both pre-Paget cells and Paget cells. In particular, AGR2 was discovered as a new marker along with known markers among the top 12 lineage driving genes of Paget cells.

The present invention provides a method of providing information for diagnosing or predicting the prognosis of extramammary Paget disease, comprising the following steps:

measuring the level of one or more selected from the group consisting of an SAM pointed domain containing ETS transcription factor (SPDEF), arginase 2 (ARG2), and adipocyte enhancer-binding protein 1 (ABEP1) from a biological sample isolated from a subject; and

comparing the measured level of one or more selected from the group consisting of SPDEF, ARG2, and ABEP1 with the level in a biological sample isolated from the control.

According to one embodiment of the present invention, the method of providing information may further comprise:

when the level of any one or more selected from the group consisting of SPDEF, ARG2, and ABEP1 in the biological sample isolated from the subject is higher than the level in the biological sample isolated from the control, determining this case to be extramammary Paget disease.

In one embodiment of the present invention, wherein the extramammary Paget's disease is non-invasive extramammary Paget's disease, or invasive extramammary Paget's disease, but is not limited thereto.

In one embodiment of the present invention, wherein the non-invasive extramammary Paget's disease is early extramammary Paget's disease, or non-early extramammary Paget's disease, but is not limited thereto.

According to one embodiment of the present invention, the method of providing information may further comprise:

when the level of ARG2 in the biological sample isolated from the subject is higher than the level in the biological sample isolated from the control, determining this case to be non-invasive extramammary Paget disease; or

when the level of ABEP1 in the biological sample isolated from the subject is higher than the level in the biological sample isolated from the control, determining this case to be invasive extramammary Paget disease, but the present invention is not limited thereto.

In the present invention, the term “non-invasive extramammary Paget disease” refers to extramammary Paget disease that is not invasive, and includes all of “extramammary Paget disease,” “baseline extramammary Paget disease,” and “early-stage extramammary Paget disease.” In this case, non-invasive extramammary Paget disease may be used interchangeably with “extramammary Paget disease” and “baseline extramammary Paget disease.” For example, in the present invention, the EMPD group may refer to a “non-invasive” EMPD group.

Therefore, in one embodiment of the present invention, the non-invasive extramammary Paget's disease may be early extramammary Paget's disease or non-early extramammary Paget's disease, but is not limited thereto.

In the present invention, the term “invasive extramammary Paget disease” may refer to extramammary Paget disease that is not in a normal state and is not non-invasive extramammary Paget disease. In particular, when confirmed histologically, invasive extramammary Paget disease is a form of extramammary Paget disease that has escaped the epidermis and invaded the dermis, and may refer to extramammary Paget disease that has become malignant. In addition, in one embodiment of the present invention, the invasive extramammary Paget's disease may be extramammary Paget's disease that persists even after extramammary Paget's disease drug administration, but is not limited thereto. In addition, it is clear that this refers to a general case diagnosed as invasive extramammary Paget's disease in the art. In the present invention, the drug administration may be performed for one year, but is not limited thereto. In the present invention, subjects treated with imiquimod for 4 months and then with ingenol mebutate for 7 months were used, but are not limited thereto.

In the present invention, “invasive” can be used interchangeably with “malignant,” but is not limited thereto.

In the present invention, “early extramammary Paget's disease” may mean extramammary Paget's disease in which pre (progenitor)-Paget cells, as defined in the present invention, are identified.

In one embodiment of the present invention, when measuring the level of one or more selected from the group consisting of SPDEF and ARG2, the control group is a normal control group; or

When measuring the level of ABEP1, the control group may be a normal control group or an individual with non-invasive extramammary Paget's disease, but is not limited thereto.

That is, in the present invention, it is confirmed that SPDEF, ARG2, and ABEP1 are each markers for diagnosis or prognosis of non-invasive extramammary Paget's disease, especially early or non-early extramammary Paget's disease, or invasive extramammary Paget's disease. Therefore, each non-invasive/invasive extramammary Paget's disease may be distinguished as an individual disease, but is not limited thereto.

According to one embodiment of the present invention, the method of providing information may further comprise:

When the biological sample isolated from the subject is a pre-Paget cell,

when the level of any one or more selected from the group consisting of SPDEF and AGR2 in the biological sample isolated from the subject is higher than the level in the biological sample isolated from the control, determining this case to early extramammary Paget's disease among non-invasive extramammary Paget's disease, but the present invention is not limited thereto.

In the present invention, pre-Paget cells are cells of the secretory system and may refer to cells that do not exhibit Paget cell lesions but express specific markers and are differentiated from normal cells. Additionally, in the present invention, pre-Paget cells may refer to cells destined to develop into Paget cells. Markers expressed in progenitor-Paget cells may include, NPY1R, AQP5, SCGB1D2, DCD, SLC12A2, and/or MUC1, but are not limited to. In particular, in the present invention, it was confirmed that AGR2 and SPDEF are expressed in pre-Paget cells.

In the present invention, the term “early-stage extramammary Paget disease” may refer to extramammary Paget disease in which pre-Paget cells defined in the present invention are identified.

In the present invention, the term “biological sample” may be included without any limitation as long as it is collected from a subject for the purpose of diagnosing extramammary Paget disease or predicting the risk of developing extramammary Paget disease. Preferably, the biological sample may be a tissue with a lesion. In the present invention, the tissue may be extramammary Paget disease tissue, but the present invention is not limited thereto.

According to one embodiment of the present invention, the biological sample may be any one selected from the group consisting of tissue, blood, serum, whole blood, plasma, urine, saliva, cells, organs, bone marrow, a fine needle aspiration specimen, a core needle biopsy specimen, and a vacuum-assisted suction biopsy specimen, but the present invention is not limited thereto.

The biological sample may be pretreated before use for detection or diagnosis. For example, the pretreatment may include homogenization, filtration, distillation, extraction, concentration, inactivation of interfering components, addition of reagents, and the like. The sample may be prepared to increase the detection sensitivity of a protein marker. For example, a sample obtained from a subject may be pretreated using methods such as anion exchange chromatography, affinity chromatography, size exclusion chromatography, liquid chromatography, sequential extraction, gel electrophoresis, or the like.

In the present invention, a method of measuring a protein level is not particularly limited as long as the method is a protein measurement method as known in the art. In this case, the protein level may be measured using methods such as protein chip analysis, an immunoassay, a ligand binding assay, matrix assisted laser desorption/ionization time of flight mass spectrometry (MALDI-TOF) analysis, surface enhanced laser desorption/ionization time of flight mass spectrometry (SELDI-TOF) analysis, a radioimmunoassay, a radial immunodiffusion method, an Ouchterlony immunodiffusion method, rocket immunoelectrophoresis, tissue immunostaining, a complement fixation assay, two-dimensional electrophoresis analysis, liquid chromatography-mass spectrometry (LC-MS), liquid chromatography-mass spectrometry/mass spectrometry (LC-MS/MS), Western blotting, an enzyme linked immunosorbent assay (ELISA), FACS, and the like.

In the present invention, a method of measuring an mRNA level is not particularly limited as long as it is an mRNA measurement method as known in the art. In this case, the mRNA level may be measured by methods such as PCR, an RNase protection assay, Northern blotting, Southern blotting, in situ hybridization, DNA chips, and/or RNA chips.

In this specification, the term “increased level” means what was not detected is detected, or that the detection amount is relatively higher than the normal level. For example, an “increased” level means that the level in an experimental group is at least 1%, 2%, 3%, 4%, 5%, 10% or higher, such as 5%, 10%, 20%, 30%, 40%, or 50%, 60%, 70%, 80%, 90% or higher, and/or 0.5-fold, 1.1-fold, 1.2-fold, 1.4-fold, 1.6-fold, 1.8-fold or higher compared to that of the control. Specifically, the increased level may mean that the level in an experimental group is 1 to 1.5-fold, 1.5 to 2-fold, 2 to 2.5-fold, 2.5 to 3-fold, 3 to 3.5-fold, 3.5 to 4-fold, 4 to 4.5-fold, 4.5 to 5-fold, 5 to 5.5-fold, 5.5 to 6-fold, 6 to 6.5-fold, 6.5 to 7-fold, 7 to 7.5-fold, 7.5 to 8-fold, 8 to 8.5-fold, 8.5 to 9-fold, 9 to 9.5-fold, 9.5 to 10-fold, or 10-fold or higher compared to that of the control, but the present invention is not limited thereto. In this case, the meaning of the opposite term thereof can be understood by those skilled in the art as having the opposite meaning according to the above definition.

As used in the present invention, the term “method of providing information” refers to a method of providing information regarding the diagnosis of a disease, such as a method of analyzing a biological sample of a subject or checking an increase or decrease in levels of biomarkers according to the present invention to obtain information on the onset of a disease or the possibility (risk) of developing the disease. For example, the method of providing information may include methods of providing information about whether a subject is likely to develop (non-invasive/invasive) extramammary Paget disease, whether a subject has a relatively high likelihood of developing (non-invasive/invasive) extramammary Paget disease, or whether a subject has already developed (non-invasive/invasive) extramammary Paget disease, by measuring the level of the biomarker according to the present invention and comparing the level of the biomarker with that of the control. Furthermore, the above method can be used to predict the risk of developing worsening extramammary Paget disease, that is, a malignant group (high-risk group). Also, the method may be used as a method of providing information on the prevention and treatment of extramammary Paget disease.

According to one embodiment of the present invention, the control may be a normal subject or a subject with extramammary Paget disease, but the present invention is not limited thereto.

The present invention provides a method of screening for an agent for preventing or treating non-invasive or invasive extramammary Paget disease, which comprises the following steps:

measuring the level of any one or more selected from the group consisting of an SAM pointed domain containing ETS transcription factor (SPDEF), arginase 2 (ARG2), and adipocyte enhancer-binding protein 1 (ABEP1) from a biological sample isolated from an animal model of extramammary Paget disease administered a candidate material; and

when the level of any one or more selected from the group consisting of SPDEF, ARG2, and AEBP1 in the biological sample isolated from the animal model decreases, selecting the candidate material as an agent for preventing or treating extramammary Paget disease.

According to one embodiment of the present invention, the screening method may further comprise:

when the level of AGR2 in the biological sample isolated from the animal model decreases, selecting the candidate material as an agent for preventing or treating non-invasive extramammary Paget disease; or

when the level of ABEP1 in the biological sample isolated from the animal model decreases, selecting the candidate material as an agent for preventing or treating invasive extramammary Paget disease, but the present invention is not limited thereto.

In the present invention, the term “screening” may mean selecting a material with any desired specific properties from a candidate group consisting of several materials using a specific manipulation or evaluation method.

In the present invention, the term “candidate material” refers to an unknown material used in screening to measure the increase or decrease in expression of the marker of the present invention by administering the material to an animal model of extramammary Paget disease, and may be any one or more selected from the group consisting of nucleotides, DNA, RNA, amino acids, aptamers, proteins, stem cells, a stem cell culture broth, compounds, a microbial culture broth or extract, natural products, and natural extracts, but the present invention is not limited thereto.

In the present invention, the term “treatment” refers to all actions that improve or beneficially change a target disease and associated metabolic abnormalities thereof. In this case, methods such as chemotherapy, surgery, biological therapy, or the like may be used.

In the present invention, a commonly used treatment method may be used to treat extramammary Paget disease, a commonly used drug for treating extramammary Paget disease may be administered, and the candidate material disclosed in the present invention may be administered, but the present invention is not limited thereto.

The present invention provides a kit for diagnosing or predicting the prognosis of extramammary Paget disease, which comprises the following:
i) a composition comprising, as an active ingredient, an agent for measuring the level of one or more selected from the group consisting of an SAM pointed domain containing ETS transcription factor (SPDEF), arginase 2 (ARG2), and adipocyte enhancer-binding protein 1 (ABEP1); and
ii) instructions.

The present invention provides a kit for screening for an agent for preventing or treating extramammary Paget disease, which comprises the following:
i) a composition comprising, as an active ingredient, an agent for measuring the level of one or more selected from the group consisting of an SAM pointed domain containing ETS transcription factor (SPDEF), arginase 2 (ARG2), and adipocyte enhancer-binding protein 1 (ABEP1); and
ii) instructions.

In the present invention, the “kit” refers to a tool that includes an agent for measuring the levels of SPDEF, ARG2, and ABEP1 to diagnose or predict the prognosis of (non-invasive/invasive) extramammary Paget disease. In the present invention, the “kit” also refers to a tool that includes an agent for measuring the levels of SPDEF, ARG2, and ABEP1 to screen for agents for preventing or treating (non-invasive/invasive) extramammary Paget disease. In addition to the above agent, the kit of the present invention may include other components, compositions, solutions, devices, and the like commonly required for methods of measuring or detecting the levels. As a specific example, because the SPDEF, ARG2, and ABEP1 of the present invention are measured in the biological sample from the subject, the kit of the present invention may further include tools for collecting the subject's sample, components required for blood storage, management, and the like, but the present invention is not limited thereto. At this time, each of the components may be applied one or more times without any limitation, and there is no restriction on the order in which the respective materials are applied. In this case, the application of each material may be carried out simultaneously or at any time point.

In the present invention, the kit may include a container; instructions; and the like. The container may serve to package the agents and may also serve to store and secure the agents. The material of the container may take the form of, for example, a bottle, a tub, a sachet, an envelope, a tube, an ampoule, and the like, which may be partially or entirely formed of plastic, glass, paper, foil, wax, and the like. The container may be equipped with a completely or partially removable cover that may initially be part of the container or may be attached to the container by mechanical, adhesive, or other means and may also be equipped with a stopper which allows access to the contents by a syringe needle. The kit may include an external package, and the external package may include instructions for use of the components.

Also, the present invention may provide a device for diagnosing non-invasive/invasive extramammary Paget disease in a subject. A measurement unit of the diagnostic device of the present invention may be configured to measure the expression level of a protein or gene using an agent for measuring the levels of the biomarkers, SPDEF, ARG2, and ABEP1 according to the present invention in a biological sample (e.g., blood or the like) obtained from the subject. By determining the expression level of the protein or gene using the agents in the measurement unit, (non-invasive/invasive) extramammary Paget disease may be diagnosed, or (non-invasive/invasive) extramammary Paget disease may be diagnosed as having a high risk of development.

The diagnostic device of the present invention may further include a detection unit configured to predict and output the presence, stage, or type of (non-invasive/invasive) extramammary Paget disease of the subject based on the expression level of the protein or gene obtained in the measurement unit.

In the present invention, the detection unit may diagnose (non-invasive/invasive) extramammary Paget disease by generating and classifying information on (non-invasive/invasive) extramammary Paget disease according to the category of expression level of the protein or gene obtained from the measurement unit.

Also, the present invention provides a method of preventing or treating extramammary Paget disease, which comprises the following steps:
measuring the level of one or more selected from the group consisting of an SAM pointed domain containing ETS transcription factor (SPDEF), arginase 2 (ARG2), and adipocyte enhancer-binding protein 1 (ABEP1) from a biological sample isolated from a subject;
comparing the measured level of one or more selected from the group consisting of SPDEF, ARG2, and ABEP1 with the level in a biological sample isolated from the control;
when the level of any one or more selected from the group consisting of SPDEF, ARG2, and ABEP1 in the biological sample isolated from the subject is higher than the level in the biological sample isolated from the control, determining this case to be extramammary Paget disease; and
administering an agent for preventing or treating extramammary Paget disease to a subject determined to have extramammary Paget disease.

According to one embodiment of the present invention, the method of preventing or treating extramammary Paget disease may further comprise:
when the level of ARG2 in the biological sample isolated from the subject is higher than the level in the biological sample isolated from the control, determining this case to be non-invasive extramammary Paget disease; or
when the level of ABEP1 in the biological sample isolated from the subject is higher than the level in the biological sample isolated from the control, determining this case to be invasive extramammary Paget disease,
administering an agent for preventing or treating non-invasive extramammary Paget disease or invasive extramammary Paget disease to a subject determined to have non-invasive extramammary Paget disease or invasive extramammary Paget disease respectively, but the present invention is not limited thereto.

According to one embodiment of the present invention, the method of preventing or treating extramammary Paget disease may further comprise:

When the biological sample isolated from the subject is a pre-Paget cell,
when the level of any one or more selected from the group consisting of SPDEF and AGR2 in the biological sample isolated from the subject is higher than the level in the biological sample isolated from the control, determining this case to early extramammary Paget's disease among non-invasive extramammary Paget's disease;
administering an agent for preventing or treating early extramammary Paget disease to a subject determined to have early extramammary Paget disease, but the present invention is not limited thereto.

In addition, the present invention provides a marker for treating EMPD, in particular, non-invasive extramammary Paget disease or invasive extramammary Paget disease, which comprises any one or more selected from the group consisting of an SAM pointed domain containing ETS transcription factor (SPDEF), arginase 2 (ARG2), and adipocyte enhancer-binding protein 1 (ABEP1).

In the present invention, because the SPDEF, ARG2, and ABEP1 are characterized as being increased in a subject with extramammary Paget disease, this increasing pattern may be used to diagnose or predict the prognosis of (non-invasive/invasive) extramammary Paget disease and may also be used as a target for the treatment of (non-invasive/invasive) extramammary Paget disease.

Furthermore, the present invention provides a composition for diagnosing or predicting the prognosis of normal, EMPD, in particular, non-invasive extramammary Paget disease, or invasive extramammary Paget disease, which includes, as an active ingredient, an agent for measuring the level of any one or more selected from the group consisting of an SAM pointed domain containing ETS transcription factor (SPDEF), arginase 2 (ARG2), and adipocyte enhancer-binding protein 1 (ABEP1).

Further, the present invention provides a composition for screening for a material for treating EMPD, in particular, non-invasive extramammary Paget disease or invasive extramammary Paget disease, which includes, as an active ingredient, an agent for measuring the level of any one or more selected from the group consisting of an SAM pointed domain containing ETS transcription factor (SPDEF), arginase 2 (ARG2), and adipocyte enhancer-binding protein 1 (ABEP1).

That is, any one or more selected from the group consisting of an SAM pointed domain containing ETS transcription factor (SPDEF), arginase 2 (ARG2), and adipocyte enhancer-binding protein 1 (ABEP1); particularly, SPDEF, ARG2, and ABEP1 may be used as biomarkers for diagnosing or predicting the prognosis of (non-invasive/invasive) extramammary Paget disease. More preferably, SPDEF and ARG2 may be used as biomarkers for diagnosing or predicting the prognosis of non-invasive extramammary Paget disease. Also, ABEP1 may be used as a biomarker for diagnosing or predicting the prognosis of invasive extramammary Paget disease.

In the present invention, the term “biomarker” refers to a marker that may distinguish between normal and pathological conditions or predict a treatment response and may be objectively measured. In this case, each of the levels of SPDEF, ARG2, and ABEP1 in the biological sample from the subject with (non-invasive/invasive) extramammary Paget disease according to the present invention was confirmed to be different from the increase or decrease in each level compared to the normal control or (non-invasive/invasive) extramammary Paget disease. Therefore, it has been proven that SPDEF, ARG2, and ABEP1 may be used as biomarkers for diagnosing or predicting the prognosis of (non-invasive/invasive) extramammary Paget disease.

In the present invention, the term “diagnosis” refers to a process of confirming the presence or characteristics of a pathological condition. For the purposes of the present invention, diagnosis is confirming the presence, occurrence, or likelihood of developing (non-invasive/invasive) extramammary Paget disease, but the present invention is not limited thereto. In this case, diagnosis includes all processes of confirming the severity of (non-invasive/invasive) extramammary Paget disease.

More specifically, as used in the present invention, the term “diagnosis” includes determining the susceptibility of a subject to a specific disease or disorder, determining whether a subject currently has a specific disease or disorder, determining the prognosis (e.g., identifying the tumor status, determining the stage of a tumor, or determining the responsiveness of a cancer to treatment) of a subject suffering from a specific disease or disorder, or therametrics (e.g., monitoring the status of the subject to provide information on the treatment efficacy).

In the present invention, the term “measurement” is intended to include both a process of detecting and determining the presence (expression) of a target material, or a process of detecting and determining a change in the presence level (expression level) of a target material. The measurement may be performed without any limitation and includes both qualitative methods (analysis) and quantitative methods. The types of qualitative and quantitative methods for measuring the presence or absence of the material of the present invention are well known in the art, and include the experimental methods described herein.

As used in this specification, the term “analysis” may preferably mean “measurement,” the qualitative analysis may mean a process of measuring and determining the presence of the target material, and the quantitative analysis may mean a process of measuring and determining changes in the presence level (expression level) or amount of the target material. In the present invention, analysis or measurement may be performed without any limitation and includes both qualitative and quantitative methods. Preferably, quantitative measurement may be performed.

In the present invention, the term “prognosis prediction” may refer to a process of predicting the degree of disease progression in a group of patients with (non-invasive/invasive) extramammary Paget disease. This may mean that the probability of progression, worsening, recurrence, maintenance, and the like of symptoms of (non-invasive/invasive) extramammary Paget disease is predicted through the increase or decrease in the levels of the biomarkers of the present invention.

In the present invention, the term “subject” refers to a subject who requires the risk prediction, diagnosis, prognosis prediction, or treatment of a disease, and more specifically, a mammal such as a human or non-human primate, a mouse, a rat, a dog, a cat, a horse, a cow, and the like, but the present invention is not limited thereto.

In the present invention, when the term “including” is used, the term means that other components may be further included rather than excluding other components unless specifically stated to the contrary. As used throughout the present invention, the term “step of” does not refer to “step for.”

Hereinafter, preferred examples of the present invention are presented in order to aid in understanding the present invention. However, it should be understood that the following examples are provided only to make the present invention easier to understand and are not intended to limit the present invention.

### EXAMPLES

### Ethics Statement

The study protocol was approved by the Institutional Review Board of Uijeongbu St. Mary's Hospital (UC17TNSI0078). The present inventors certify that all applicable institutional regulations regarding the ethical use of patients' information and samples were strictly followed in this work.

### Human Sample

Biological samples for this study were provided by the Uijeongbu St. Mary's Hospital Biobank of the Catholic University of Korea. A 63-year-old man had an undefined red spot in his right groin. A skin biopsy showed scattered Paget cells in the epidermis with abundant cytoplasm and clear nuclei. The patient preferred topical treatment because the patient had a small skin lesion and showed only mild erythema. The patient was treated with imiquimod, which was applied 3 times a week at 2-week intervals for 4 months. Thereafter, the patient was treated with ingenol mebutate at 2-week intervals for 7 months. There was clinical improvement as erythema decreased and the size of the lesion decreased during local treatment. Skin biopsies were performed at five different sites along the border of the erythema at baseline, 4 months, 6 months, and one year after starting treatment. EMPD tissues obtained through the skin biopsies at various time points were fixed with formalin and embedded in paraffin for further analysis.

### Digital Spatial Profiling Processing and Analysis

Spatial profiles were obtained using a combination of a GeoMx® digital spatial profiler utilizing digital barcoding technology and a WTA kit (Nanostring, Seattle, WA, USA) according to the manufacturer's instructions. Selection of a region of interest (ROI) was based on the entire slide at 4× magnification and three markers (CD45, pan-cytokeratin (PanCK), and SYTO13, all provided by Nanostring). Thereafter, a sequencing library was prepared by attaching ROI-specific Illumina adapter sequences, and unique i5 and i7 sample indices were attached. Sequencing was performed using Novaseq 6000 with a sequencing depth of 100. FASTQ files were converted to a digital count conversion format using the Digital Spatial Profiler data analysis suite and subjected to quality control. To define a gene filter, the minimum number of nuclei was set to 20, the limit of quantification was set to 2, and the gene detection rate was set to 5%. Raw counts were normalized with the edgeR package. Principal component analysis (PCA) plots and Venn diagrams were generated using the ggplot2 and ggVennDiagram packages. Alluvial diagrams and correlation plots were visualized using the alluvium and coplot packages. Differentially expressed gene (DEG) analysis was performed using the limma package. DEGs were called based on log₂ fold change ≥1.0 and false discovery rate (FDR)<0.05. Heatmaps were generated using the z-scores-based ComplexHeatmap R package. Gene ontology (GO) analysis was performed using the Cluster Profiler Bioconductor package using the gseGO function. Detailed pathway analysis was performed using the web-based portal Metascape, and g: Profiler. Gene and protein networks and enriched pathways were visualized using Gene Set Enrichment Analysis (GSEA) software v4.3.2. EMPD pathway scores were assigned using the ssGSEA function of the GSVA package. Transcription factors were identified using the web-based portal ChEA3. Chea3 is a method of obtaining transcription factors using various library references. Examples of library references include ENCODE, chip data, enricher data, GTEx, etc. Graphic figures are adapted from ‘Epithelial to Mesenchymal Transition (Layout)’ and ‘Spatial Transcriptomic’ by Biorender.com (2024): http://app.biorender.com/biorender-templates.

### Spatial Transcript Deconvolution

Single-cell deconvolution was performed using CIVERSORTx, and a validated leukocyte gene signature matrix consisting of 547 genes that serves to distinguish 22 human hematopoietic subsets present in peripheral blood under in vitro culture conditions was used. Mixed data was presented using human genome symbols, and each piece of data consisted of normalized “transcript per million” values. Normalized data was analyzed with 100 permutations.

### Statistical Analysis

Data was visualized and analyzed as mean±standard deviation using R version 4.3.2 and Prism Graphpad software v.6.0 (Graphpad Software, Boston, MA). The Pearson correlation coefficient was used to determine the relationship between two genes. Each sample was tested using the Shapiro-Wilk test to determine the Gaussian distribution. For samples that met these criteria, one-way analysis of variance (ANOVA) and Tukey's multiple comparisons were used, and for samples that did not meet these criteria, the non-parametric Kruskal-Wallis test and Dunn's multiple comparisons were used. Significance levels were defined as *P<0.05, **P<0.01, ***P<0.001, and ****P<0.0001.

### Single-Cell RNA Transcriptomic Analysis

Excisional biopsy skin samples from the same EMPD patient with tumor site and lesion were surgically collected at the Uijeongbu St. Mary's Hospital. After surgery, tumor tissue was collected from the center of the lesion, and normal tissue around the lesion was collected from a 5 cm surgical margin and delivered fresh to the Seoul National University College of Medicine laboratory for dissociation within 2 hours. After cell dissociation, scRNA-seq was performed using the 10X Genomics 5′ R2-only Chemistry kit aligned with the GRCh38-2020-A reference genome. A count matrix was generated by applying the Cellranger-6.1.2 version pipeline. Peripheral RNA was removed from the raw count matrix before preprocessing through Cellbender. The default settings of scvi-tools were utilized for double filtering (scvi-Solo) and sample integration. Preprocessing, quality control, and visualization were performed in Scanpy. After quality control, 12,185 EMPD cells and 10,255 perilesional cells were obtained. A total of 22,440 cells were classified into 23 cell types.

### Example 1: Spatial Gene Expression Analysis and Classification of EMPD Samples

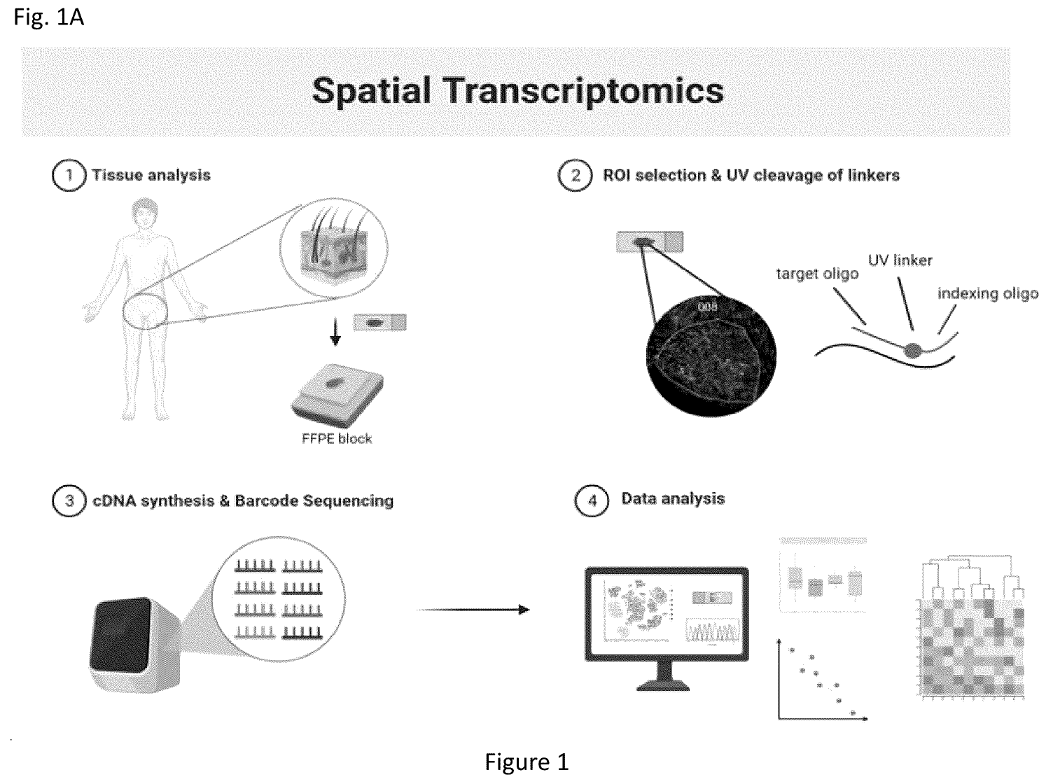

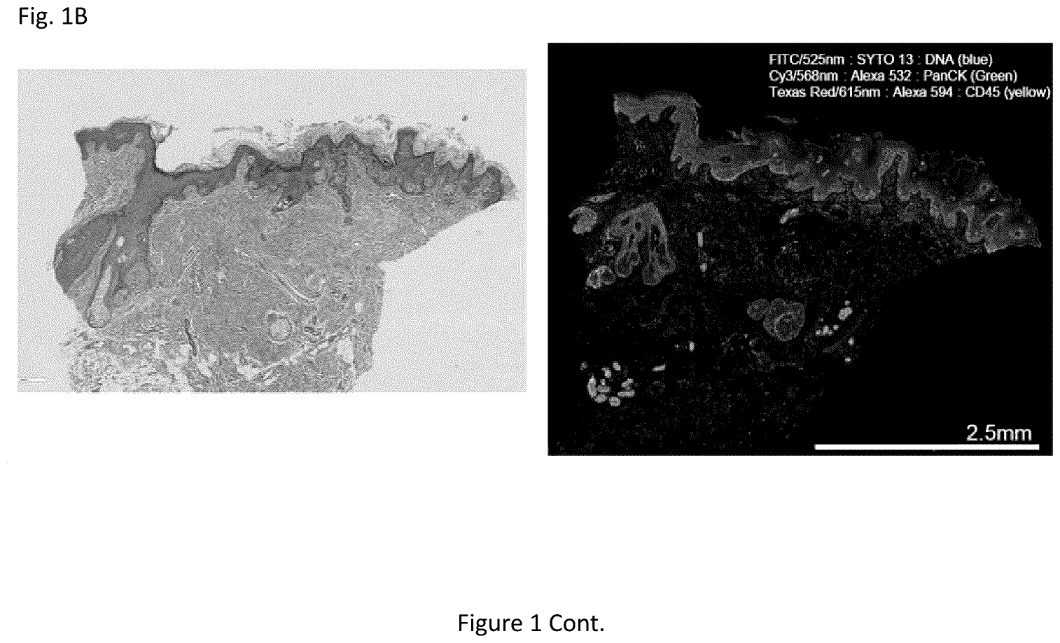

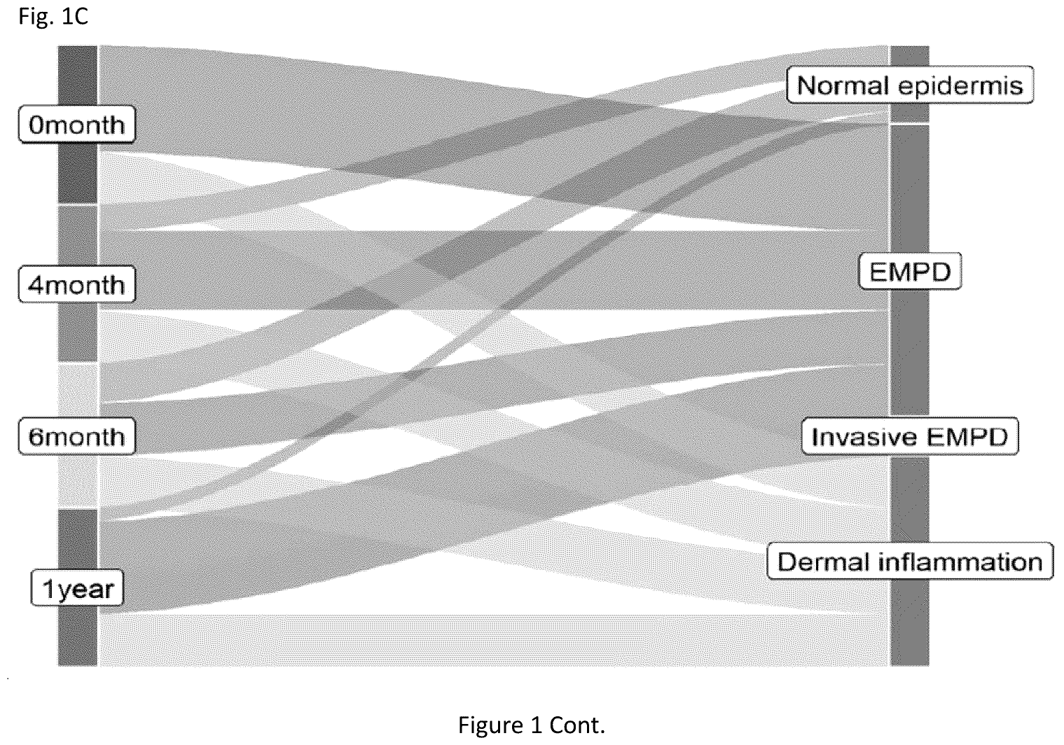

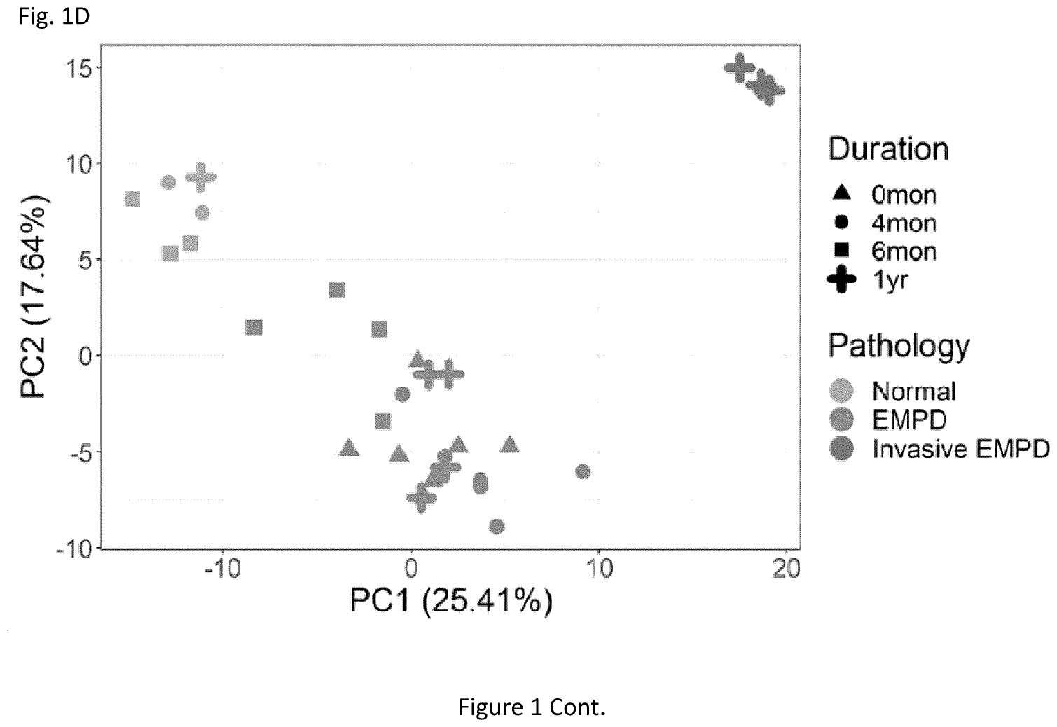

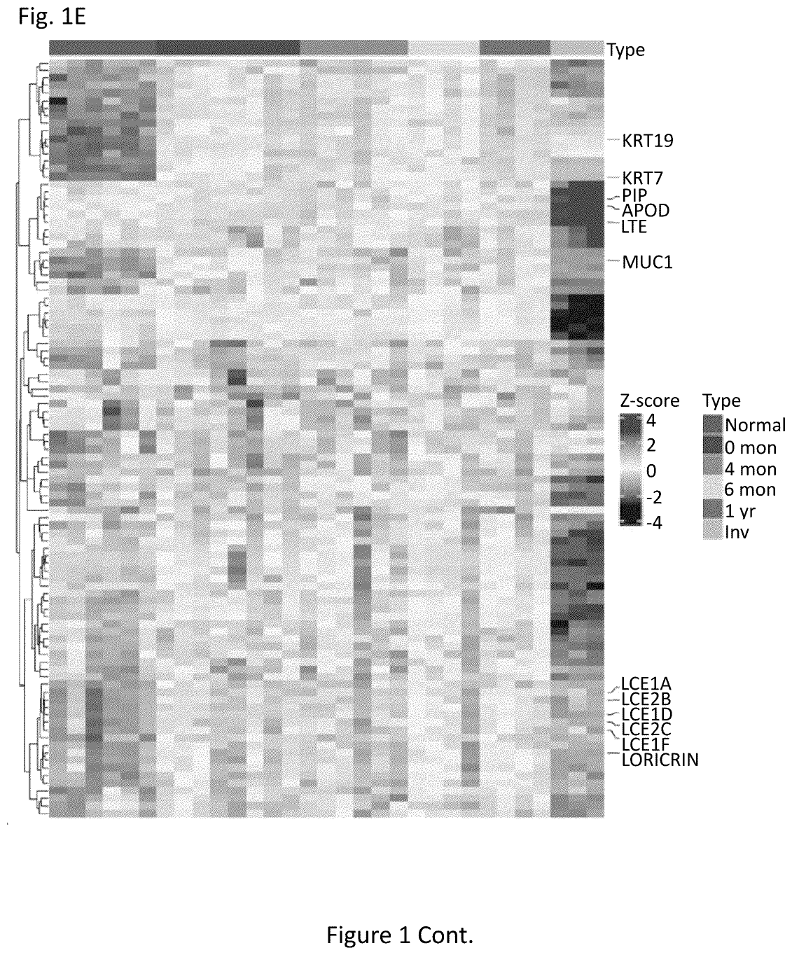

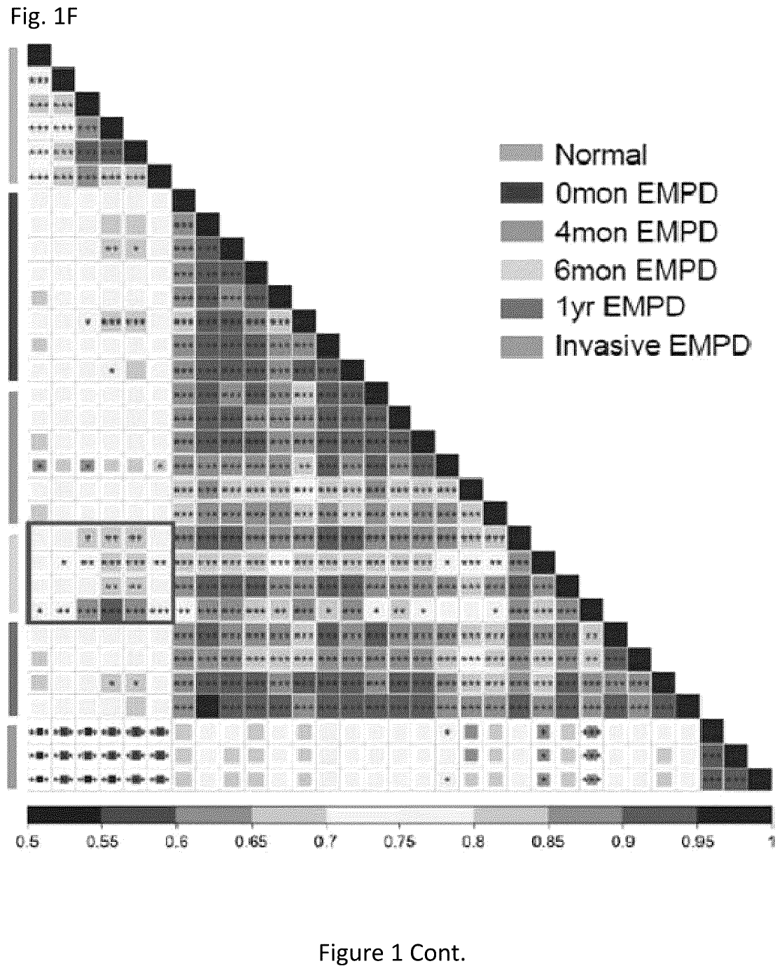

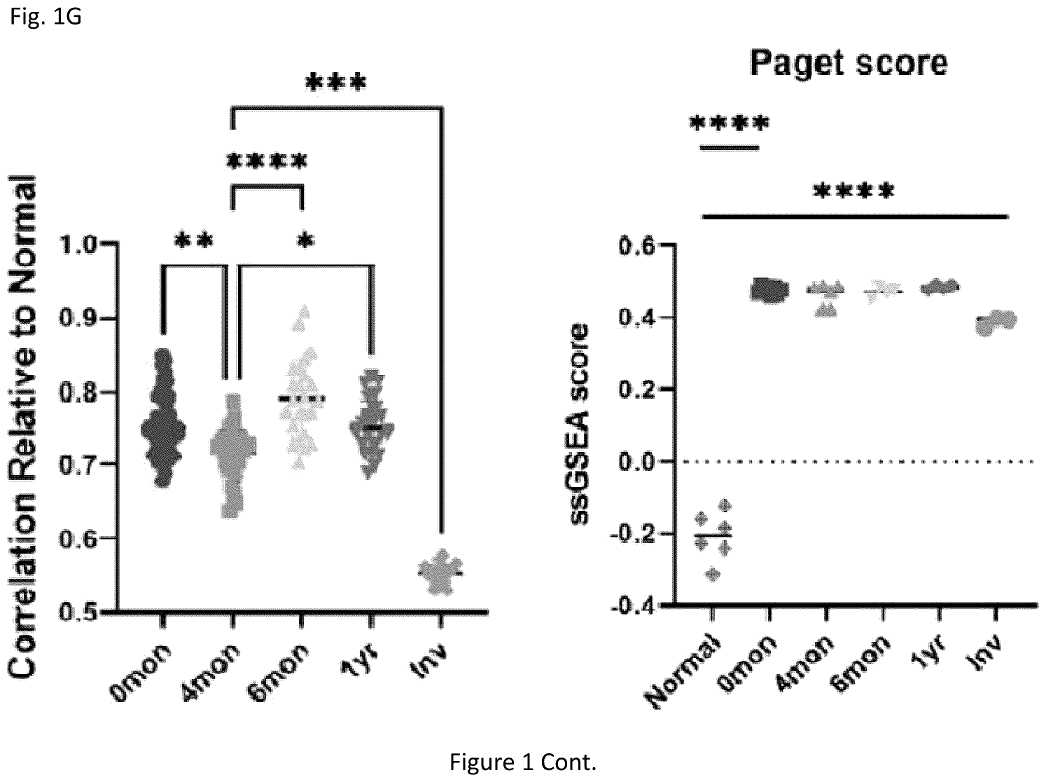

Spatial gene expression analysis at the single cell level in tissue samples has been facilitated by an in situ hybridization platform with probes. This method involves relabeling captured targets with unique barcodes that encode spatial information, which makes it possible to precisely identify RNA molecules and their locations within tissue (FIG. 1A).

The EMPD patient whose samples were used was treated using topical imiquimod for 4 months and ingenol mebutate after 7 months. Although there were some improvement at 4 and 6 months, Paget cells persisted in the epidermis despite the topical treatment. One year after treatment, histological invasion was observed for the first time, leading to discontinuation of local treatment and referral for Mohs surgery. Skin samples from the 63-year-old man were taken from five different sites along the border of the EMPD lesion 4 months, 6 months, and one year after the start of treatment. A total of 47 regions of interest (ROIs) were used in this study. Based on histological evaluation, 31 epithelial ROIs and 16 peripheral skin inflammation ROIs were profiled (FIG. 1B). ROIs with prominent Paget cells observed at each time point were defined as the EMPD group (8 ROIs at baseline, 6 ROIs at 4 months, 4 ROIs at 6 months, and 4 ROIs at one year). ROIs from areas observed to have normal epidermis during treatment were selected as the normal group (2 ROIs at 4 months, 3 ROIs at 6 months, and 1 ROI at one year). Also, three ROIs observed as being invasive at one year were separately designated as invasive EMPD. Areas of skin inflammation around EMPD observed at each time point were included in the inflammation group (4 ROIs at baseline, 4 ROIs at 4 months, 4 ROIs at 6 months, and 4 ROIs at one year) (Table 1, FIG. 1C).

TABLE 1

| ROI ID | Histology Class | Pathology |
|---|---|---|
| Skin 1.A1 | 6 month | Normal |
| Skin 1.A2 | 6 month | Normal |
| Skin 1.A3 | 6 month | Normal |
| Skin 1.A4 | 6 month | EMPD |
| Skin 1.A5 | 6 month | EMPD |
| Skin 1.A6 | 6 month | EMPD |
| Skin 1.A7 | 6 month | EMPD |
| Skin 1.B1 | 1 year | Normal |
| Skin 1.B2 | 1 year | EMPD |
| Skin 1.B3 | 1 year | EMPD |
| Skin 1.B4 | 1 year | EMPD |
| Skin 1.B5 | 1 year | EMPD |
| Skin 1.B6 | 1 year | Invasion |
| Skin 1.B7 | 1 year | Invasion |
| Skin 1.B8 | 1 year | Invasion |
| Skin 1.C1 | 4 month | Normal |
| Skin 1.C2 | 4 month | Normal |
| Skin 1.C3 | 4 month | EMPD |
| Skin 1.C4 | 4 month | EMPD |
| Skin 1.C5 | 4 month | EMPD |
| Skin 1.C6 | 4 month | EMPD |
| Skin 1.C7 | 4 month | EMPD |
| Skin 1.C8 | 4 month | EMPD |
| Skin 1.D1 | 0 month (Baseline) | EMPD |
| Skin 1.D2 | 0 month (Baseline) | EMPD |
| Skin 1.D3 | 0 month (Baseline) | EMPD |
| Skin 1.D4 | 0 month (Baseline) | EMPD |
| Skin 1.D5 | 0 month (Baseline) | EMPD |
| Skin 1.D6 | 0 month (Baseline) | EMPD |
| Skin 1.D7 | 0 month (Baseline) | EMPD |
| Skin 1.D8 | 0 month (Baseline) | EMPD |
| Skin 2.A1 | 6 month | Inflammation |
| Skin 2.A2 | 6 month | Inflammation |
| Skin 2.A3 | 6 month (QC fail) | Inflammation |
| Skin 2.A4 | 6 month | Inflammation |
| Skin 2.A5 | 6 month | Inflammation |
| Skin 2.B1 | 1 year | Inflammation |
| Skin 2.B2 | 1 year | Inflammation |
| Skin 2.B3 | 1 year | Inflammation |
| Skin 2.B4 | 1 year | Inflammation |
| Skin 2.C1 | 4 month | Inflammation |
| Skin 2.C2 | 4 month | Inflammation |
| Skin 2.C3 | 4 month | Inflammation |
| Skin 2.C4 | 4 month | Inflammation |
| Skin 2.D1 | 0 month (Baseline) | Inflammation |
| Skin 2.D2 | 0 month (Baseline) | Inflammation |
| Skin 2.D3 | 0 month (Baseline) | Inflammation |
| Skin 2.D4 | 0 month (Baseline) | Inflammation |

The samples showed distinct clustering patterns, which separated the samples into three different groups (FIG. 1D). This separation was further confirmed by identifying 100 variable genes in each sample (FIG. 1E).

In particular, the 6-month EMPD sample showed a shift toward the normal group in terms of similarity, but showed a shift away from the normal group after 1 year. A high correlation between baseline and 6-month outcomes was also identified in FIG. 1D, as marked by the red boxes. Moreover, the invasive EMPD group had significantly lower similarity to the normal group (FIG. 1F). In this study, the correlation values varied depending on the treatment period. EMPD at 6 months showed a higher correlation value with the normal group than EMPD at 4 months. Invasive EMPD showed the greatest difference with the normal samples, showing a correlation value of approximately 0.5.

To evaluate the EMPD characteristics of the sample based on ‘Known Paget Cell Marker 1’ and ‘Known Paget Cell Marker 2,’ a ‘Paget score’ was set using ssGSEA. ‘Known Paget Cell Marker 1,’ including genes such as KRT19, KRT8, KRT18, PDPN, GATA3, MYD88, FOXA1, and the like, was excluded when differentially expressed in invasive EMPD. ‘Known Paget Cell Marker 2’ such as AR, MUC1, KRT7, VIM, PIP, and the like represents a gene up-regulated in invasive EMPD.

As a result of the analysis, ‘Known Paget Cell Marker 1’ was consistently higher in EMPD compared to normal. On the other hand, the known ‘Known Paget Cell Marker 2’ varied depending on the treatment period, but showed a significant increase in invasive EMPD (FIG. 1G).

In conclusion, there were distinct phenotypes for the normal, EMPD, and invasive EMPD samples. Within EMPD, the 6-month sample showed transcriptomic improvement, which contrasted with the less favorable profile at 4 months and one year. These results suggest that EMPD and invasive EMPD are related but may exhibit unique characteristics based on established biomarkers.

### Example 2: Effect of EMPD Progression on Histone and Cell Cycle Gene Expression and Inflammatory Response

### Example 2-1: Effect of EMPD Progression on Histone and Cell Cycle Gene Expression

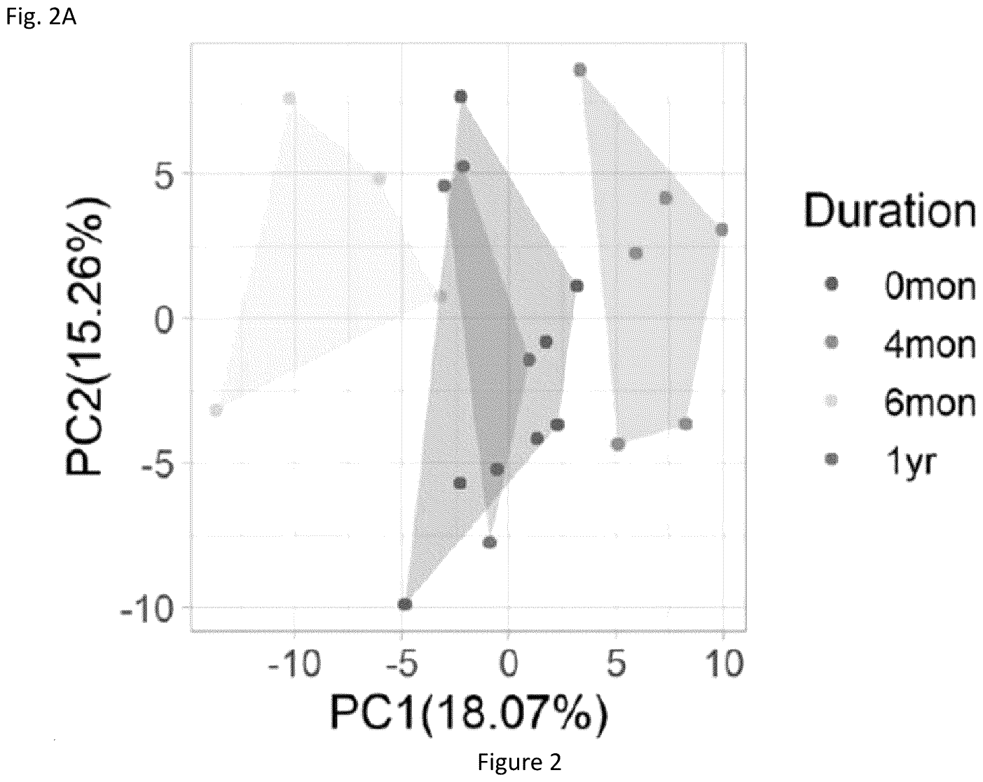

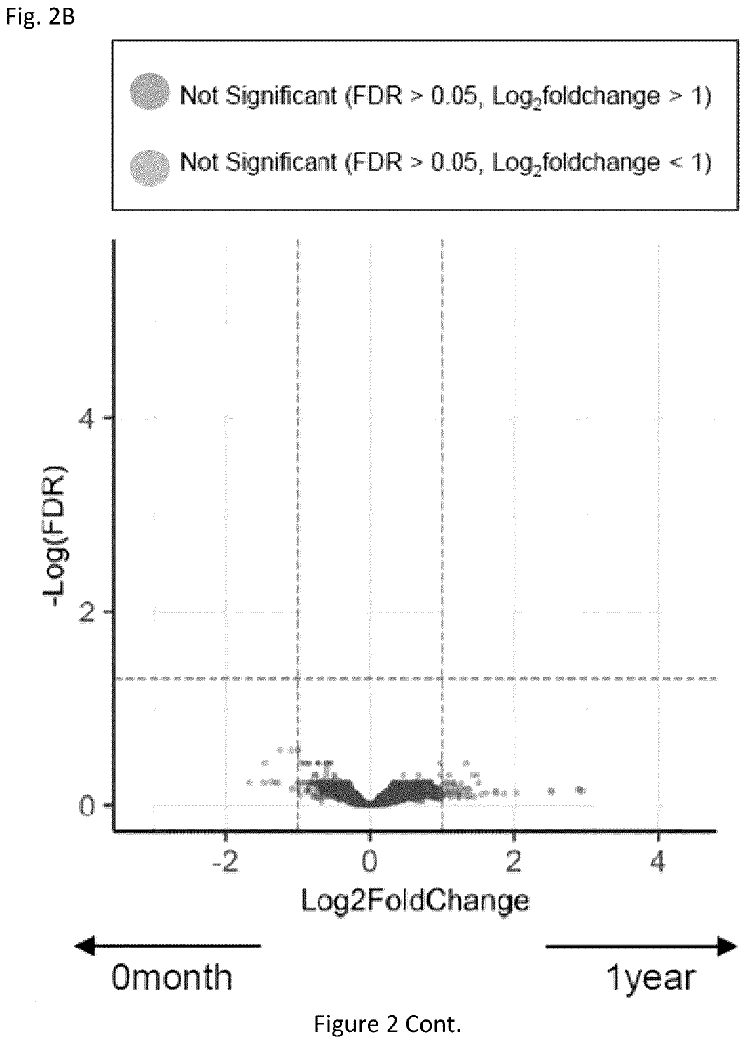

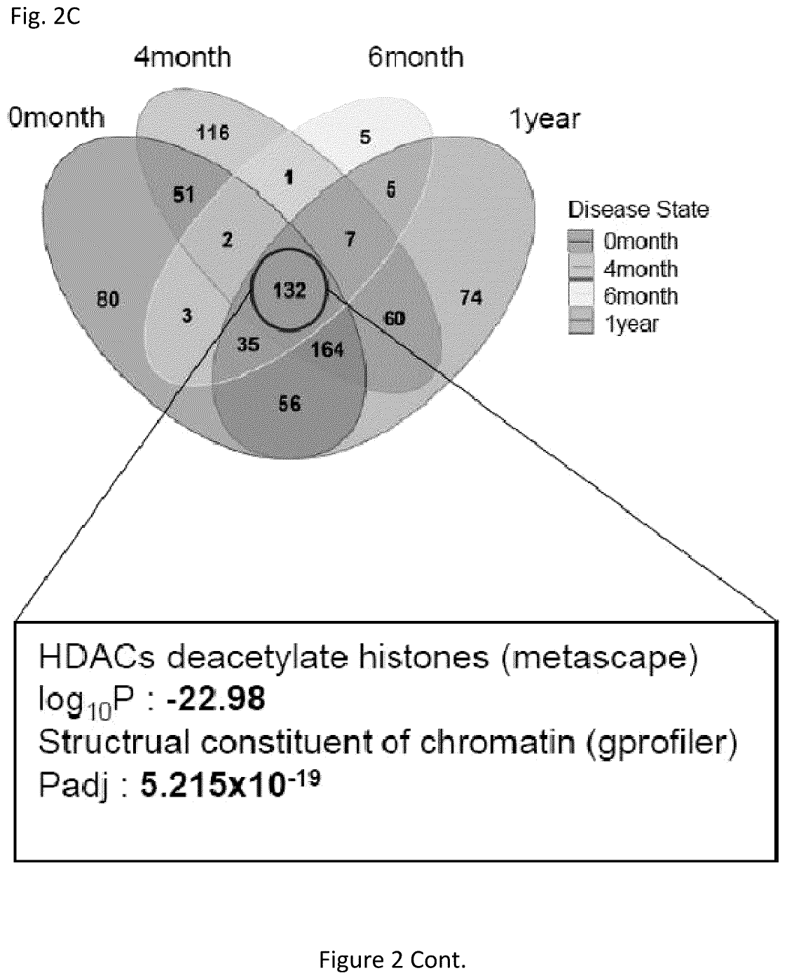

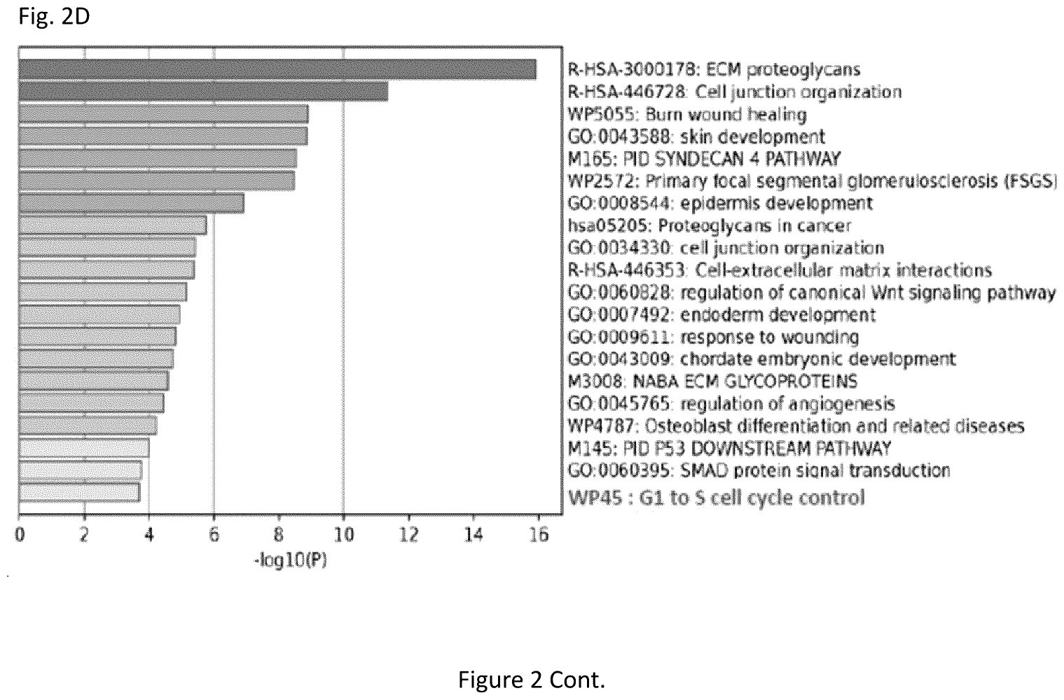

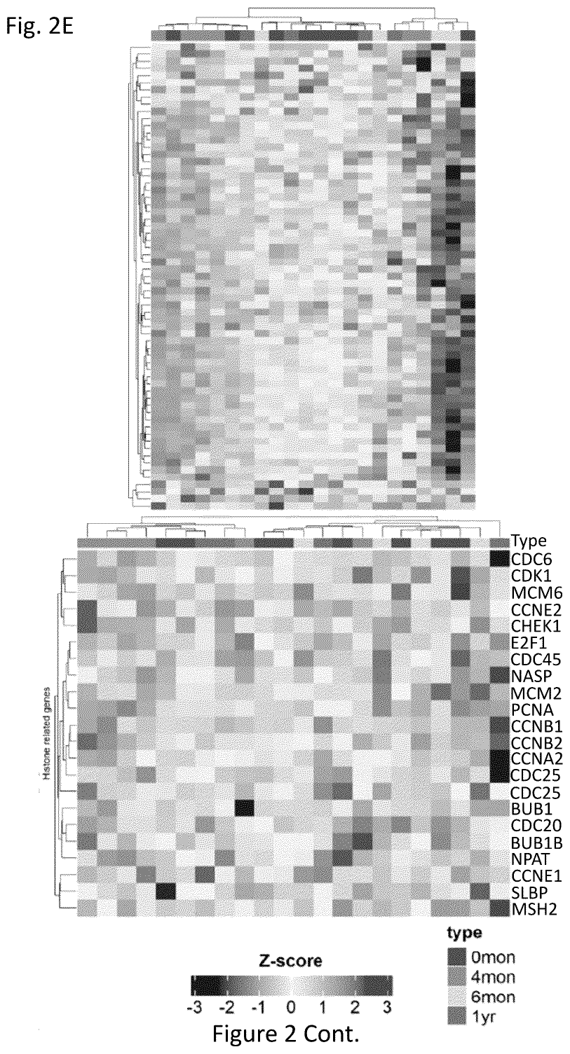

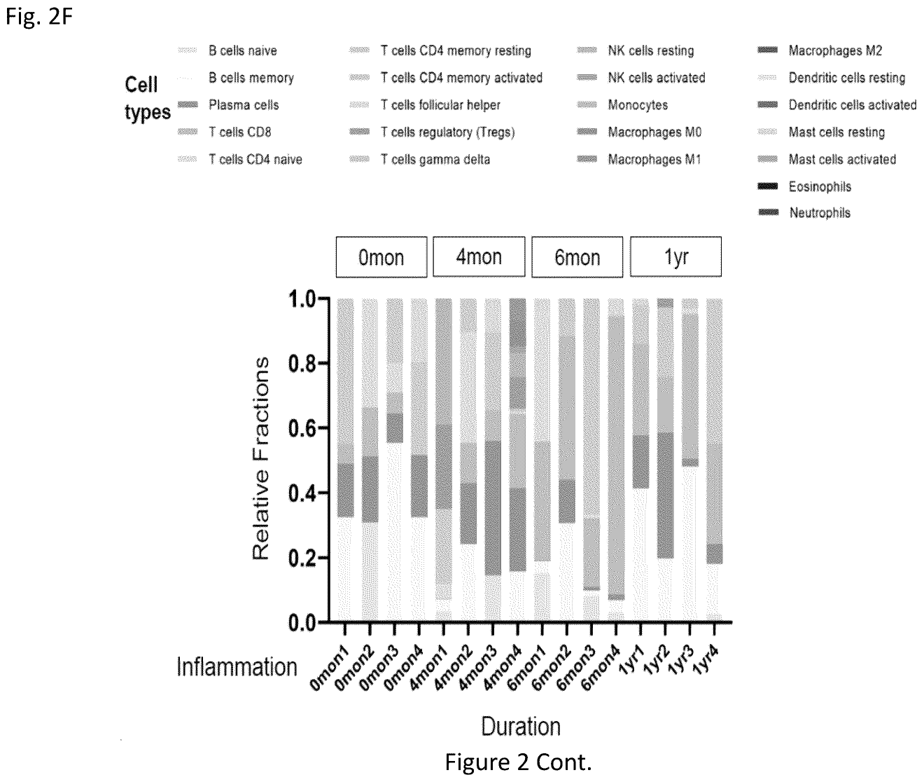

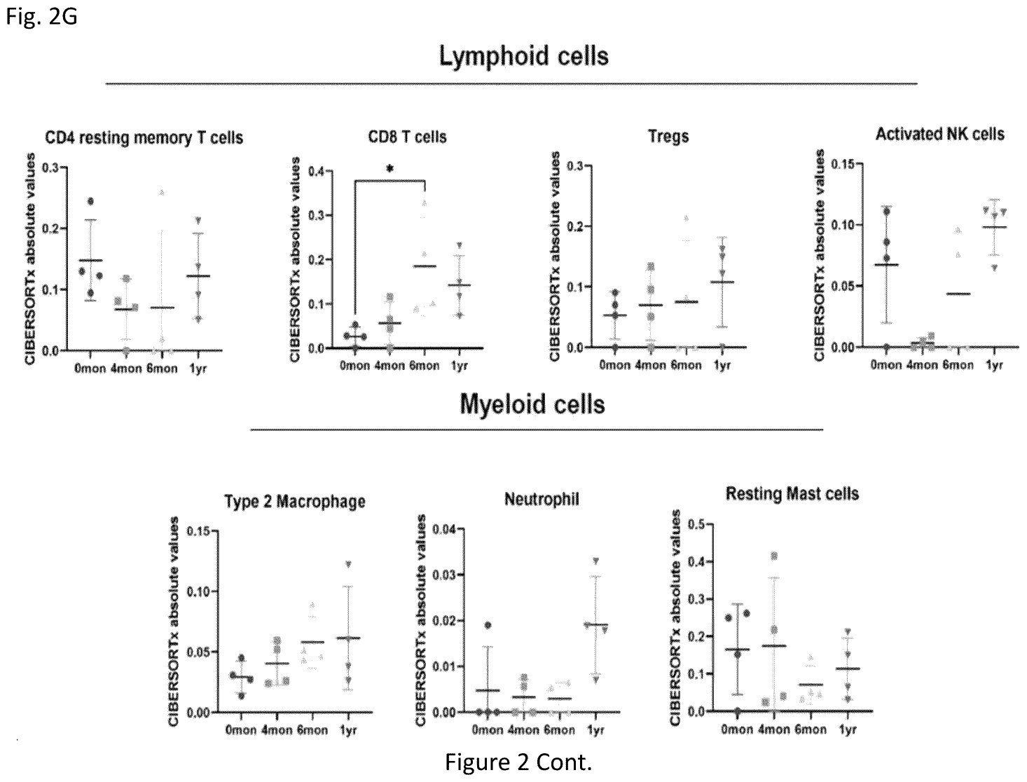

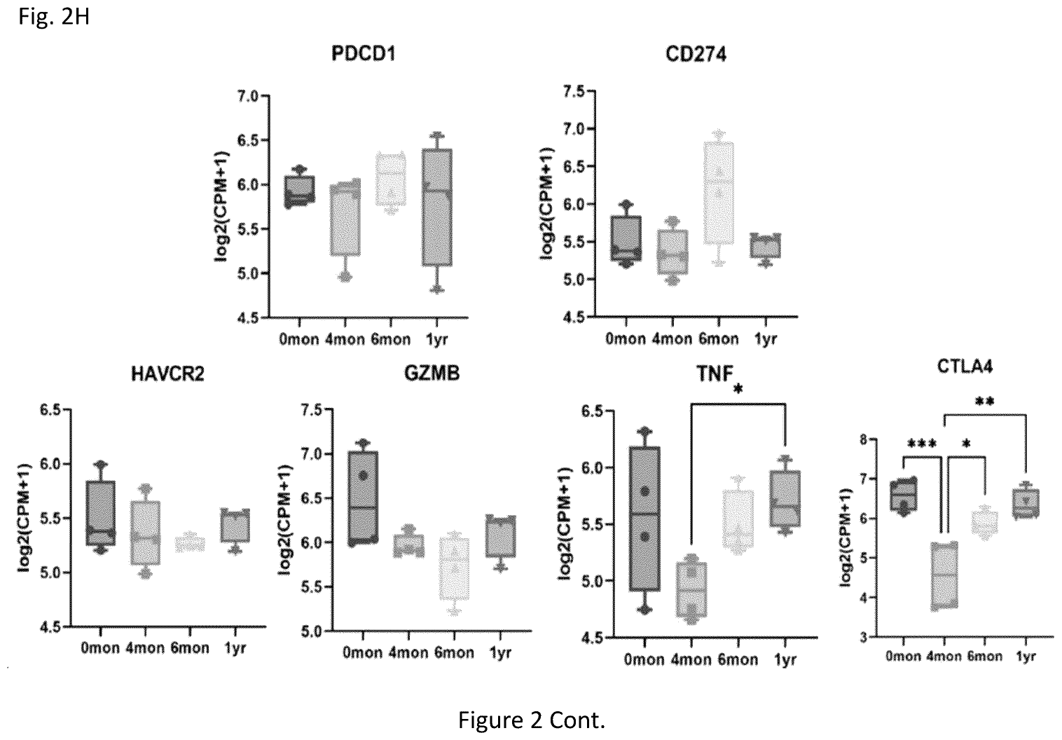

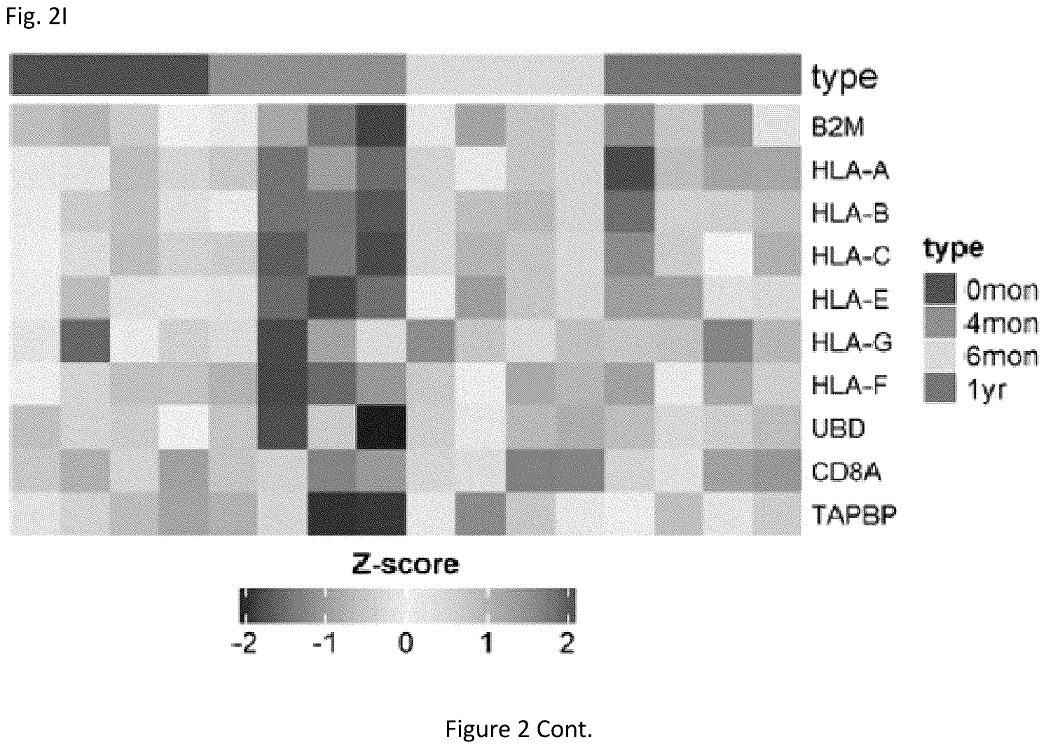

To further examine changes within the EMPD group at various time points, PCA plots were used as a comprehensive framework for differentiation (FIG. 2A). Differences were observed in the markers at 4 months and 6 months based on the results shown in the previous correlation plot (FIG. 1F).

As a result, this difference was also clearly revealed in the volcano plot comparing baseline EMPD with EMPD 1 year after treatment, highlighting major changes (log 2 fold change >1; FDR<0.05) (FIG. 2B).

Also, important genetic mutations were analyzed to distinguish differences in gene expression over time between the normal and EMPD groups.

As a result, when compared to the normal sample, there were 362 DEGs in the baseline EMPD, 609 DEGs in the 4-month EMPD, 172 DEGs in the 6-month EMPD, and 528 DEGs in the 1-year EMPD. A total of 151 genes were common across all time periods. Using Metascape and g: Profiler, the present inventors identified pathways with the two most significant p values for HDACs deacetylating histone (log P=−23.08) and ‘structural components of chromatin’ (padj=1.103×10-20) (FIG. 2C, Table 2). The key observation was the consistent presence of histone-related GOs in all EMPD samples.

TABLE 2

| | | | | | | T cells CD4 |
|---|---|---|---|---|---|---|
| | B cells | B cells | | | T cells CD4 | memory |
| Mixture | naive | memory | Plasma cells | T cells CD8 | naive | resting |
| 0 month_{—} | 0 | 0.135935 | 0.069026 | 0.025815 | 0 | 0.244551 |
| Inflammation1 | | | | | | |
| 0 month_{—} | 0.10903 | 0 | 0.072073 | 0.053298 | 0.180845 | 0.094567 |
| Inflammation2 | | | | | | |
| 0 month_{—} | 0 | 0.244027 | 0.039954 | 0.028284 | 0.03958 | 0.129954 |
| Inflammation3 | | | | | | |
| 0 month_{—} | 0 | 0.138946 | 0.082125 | 0 | 0 | 0.122997 |
| Inflammation4 | | | | | | |
| 4 month_{—} | 0.017195 | 0.018895 | 0.001087 | 0 | 0.023425 | 0.118669 |
| Inflammation1 | | | | | | |
| 4 month_{—} | 0 | 0.125888 | 0.098811 | 0.065034 | 0.177784 | 0.081271 |
| Inflammation2 | | | | | | |
| 4 month_{—} | 0.069265 | 0 | 0.197857 | 0.044504 | 0 | 0.070893 |
| Inflammation3 | | | | | | |
| 4 month_{—} | 0 | 0.08014 | 0.132535 | 0.116438 | 0.003284 | 0 |
| Inflammation4 | | | | | | |
| 6 month_{—} | 0.03745 | 0.009414 | 0.000557 | 0.091163 | 0.099545 | 0 |
| Inflammation4 | | | | | | |
| 6 month_{—} | 0 | 0.149264 | 0.065971 | 0.215805 | 0 | 0.021391 |
| Inflammation1 | | | | | | |
| 6 month_{—} | 0.039509 | 0.008395 | 0.005883 | 0.103796 | 0.004294 | 0.261116 |
| Inflammation2 | | | | | | |
| 6 month_{—} | 0.011604 | 0.01468 | 0.007222 | 0.330388 | 0 | 0 |
| Inflammation3 | | | | | | |
| 1 year_{—} | 0 | 0.171011 | 0.067355 | 0.11694 | 0 | 0.050032 |
| Inflammation1 | | | | | | |
| 1 year_{—} | 0 | 0.083505 | 0.16427 | 0.072135 | 0 | 0.09067 |
| Inflammation2 | | | | | | |
| 1 year_{—} | 0 | 0.248932 | 0.012177 | 0.231435 | 0.009014 | 0.136527 |
| Inflammation3 | | | | | | |
| 1 year_{—} | 0.0122 | 0.073571 | 0.029271 | 0.148189 | 0 | 0.212209 |
| Inflammation4 | | | | | | |

| | T cells CD4 | T cells | T cells | | |
|---|---|---|---|---|---|
| | memory | follicular | regulatory | T cells | NK cells |
| Mixture | activated | helper | (Tregs) | gamma delta | resting |
| 0 month_{—} | 0 | 0 | 0.052904 | 0.029988 | 0 |
| Inflammation1 | | | | | |
| 0 month_{—} | 0.027692 | 0 | 0 | 0.020822 | 0.05917 |
| Inflammation2 | | | | | |
| 0 month_{—} | 0 | 0.028501 | 0.070179 | 0 | 0 |
| Inflammation3 | | | | | |
| 0 month_{—} | 0 | 0.085498 | 0.090646 | 0 | 0 |
| Inflammation4 | | | | | |
| 4 month_{—} | 0 | 0.000282 | 0.134374 | 0 | 0.222666 |
| Inflammation1 | | | | | |
| 4 month_{—} | 0.038786 | 0 | 0 | 0 | 0.202528 |
| Inflammation2 | | | | | |
| 4 month_{—} | 0.043473 | 0.060453 | 0.095458 | 0 | 0.044119 |
| Inflammation3 | | | | | |
| 4 month_{—} | 0.004869 | 0 | 0.050605 | 0 | 0.037781 |
| Inflammation4 | | | | | |
| 6 month_{—} | 0 | 0.052322 | 0.084455 | 0 | 0 |
| Inflammation4 | | | | | |
| 6 month_{—} | 0.067826 | 0.02235 | 0 | 0.121245 | 0 |
| Inflammation1 | | | | | |
| 6 month_{—} | 0.090039 | 0.015137 | 0 | 0 | 0.175154 |
| Inflammation2 | | | | | |
| 6 month_{—} | 0 | 0.077816 | 0.216125 | 0 | 0.018298 |
| Inflammation3 | | | | | |
| 1 year_{—} | 0 | 0.026146 | 0.161504 | 0.004771 | 0 |
| Inflammation1 | | | | | |
| 1 year_{—} | 0 | 0 | 0.121453 | 0 | 0 |
| Inflammation2 | | | | | |
| 1 year_{—} | 0 | 0.011042 | 0 | 0 | 0 |
| Inflammation3 | | | | | |
| 1 year_{—} | 0.000577 | 0.038798 | 0.148691 | 0 | 0 |
| Inflammation4 | | | | | |

| | | | | | | Dendritic |
|---|---|---|---|---|---|---|
| | NK cells | | Macrophage | Macrophages | Macrophages | cells |
| Mixture | activated | Monocytes | M0 | M1 | M2 | resting |
| 0 month_{—} | 0.110886 | 0.014104 | 0 | 0.016206 | 0.013726 | 0.017388 |
| Inflammation1 | | | | | | |
| 0 month_{—} | 0 | 0 | 0.004743 | 0.03364 | 0.045328 | 0 |
| Inflammation2 | | | | | | |
| 0 month_{—} | 0.086112 | 0.026717 | 0.030191 | 0.053085 | 0.030994 | 0.036692 |
| Inflammation3 | | | | | | |
| 0 month_{—} | 0.072966 | 0.024267 | 0 | 0.01638 | 0.027322 | 0 |
| Inflammation4 | | | | | | |
| 4 month_{—} | 0 | 0.049328 | 0 | 0.044795 | 0.024144 | 0.079669 |
| Inflammation1 | | | | | | |
| 4 month_{—} | 0 | 0.02296 | 0 | 0.00994 | 0.059735 | 0.011207 |
| Inflammation2 | | | | | | |
| 4 month_{—} | 0.004468 | 0.024652 | 0 | 0.0106 | 0.051938 | 0.03107 |
| Inflammation3 | | | | | | |
| 4 month_{—} | 0.009288 | 0 | 0.042796 | 0.038223 | 0.025991 | 0.024192 |
| Inflammation4 | | | | | | |
| 6 month_{—} | 0.097112 | 0 | 0.15542 | 0.174046 | 0.051735 | 0 |
| Inflammation4 | | | | | | |
| 6 month_{—} | 0 | 0 | 0 | 0.054661 | 0.089889 | 0.007392 |
| Inflammation1 | | | | | | |
| 6 month_{—} | 0 | 0 | 0.014111 | 0.056441 | 0.046914 | 0.031329 |
| Inflammation2 | | | | | | |
| 6 month_{—} | 0.076403 | 0.00977 | 0.021693 | 0.064765 | 0.043866 | 0.054626 |
| Inflammation3 | | | | | | |
| 1 year_{—} | 0.111522 | 0.017197 | 0 | 0.033421 | 0.122169 | 0.036542 |
| Inflammation1 | | | | | | |
| 1 year_{—} | 0.106705 | 0.033834 | 0 | 0.016553 | 0.05988 | 0.021114 |
| Inflammation2 | | | | | | |
| 1 year_{—} | 0.109995 | 0.030098 | 0 | 0.012888 | 0.025958 | 0.024206 |
| Inflammation3 | | | | | | |
| 1 year_{—} | 0.064389 | 0.020492 | 0.002948 | 0.010702 | 0.037732 | 0.005163 |
| Inflammation4 | | | | | | |

| | Dendritic | Mast | | | |
|---|---|---|---|---|---|
| | cells | cells | Mast cells | | |
| Mixture | activated | resting | activated | Eosinophils | Neutrophils |
| 0 month_{—} | 0.020103 | 0.24937 | 0 | 0 | 0 |
| Inflammation1 | | | | | |
| 0 month_{—} | 0.01785 | 0.26194 | 0 | 0 | 0.019002 |
| Inflammation2 | | | | | |
| 0 month_{—} | 0.002067 | 0.1522 | 0.00147 | 0 | 0 |
| Inflammation3 | | | | | |
| 0 month_{—} | 0 | 0 | 0.30214 | 0.036708 | 0 |
| Inflammation4 | | | | | |
| 4 month_{—} | 0 | 0.04074 | 0.18489 | 0.039843 | 0 |
| Inflammation1 | | | | | |
| 4 month_{—} | 0 | 0.02524 | 0.03889 | 0.03428 | 0.007651 |
| Inflammation2 | | | | | |
| 4 month_{—} | 0 | 0.21813 | 0.03313 | 0 | 0 |
| Inflammation3 | | | | | |
| 4 month_{—} | 0 | 0.41589 | 0.01234 | 0 | 0.005624 |
| Inflammation4 | | | | | |
| 6 month_{—} | 0 | 0.14678 | 0 | 0 | 0 |
| Inflammation4 | | | | | |
| 6 month_{—} | 0 | 0.04731 | 0 | 0.136896 | 0 |
| Inflammation1 | | | | | |
| 6 month_{—} | 0.001883 | 0.053 | 0.07029 | 0.017343 | 0.005369 |
| Inflammation2 | | | | | |
| 6 month_{—} | 0 | 0.03539 | 0.01076 | 0 | 0.006594 |
| Inflammation3 | | | | | |
| 1 year_{—} | 0 | 0.06361 | 0 | 0 | 0.017781 |
| Inflammation1 | | | | | |
| 1 year_{—} | 0 | 0.21111 | 0 | 0 | 0.018767 |
| Inflammation2 | | | | | |
| 1 year_{—} | 0.030593 | 0.03007 | 0.03084 | 0.023333 | 0.032893 |
| Inflammation3 | | | | | |
| 1 year_{—} | 0.010458 | 0.14926 | 0.0252 | 0.003195 | 0.006955 |
| Inflammation4 | | | | | |

To identify basic differences between the baseline and 4-month samples, the present inventors specifically compared these two treatment periods.

As a result, it was confirmed that important pathways such as cell junction tissue and proteoglycans in cancer were significantly enriched at 4 months compared to baseline EMPD. Also, the cell cycle control pathways from G1 to S were significantly correlated (FIG. 2D). Then, a heatmap was created to examine the correlation between S cell cycle genes and histone-related genes in G1 within the WP45 pathway, focusing on the 4-month time point (FIG. 2E). These results supported the hypothesis by revealing significant up-regulation of the S cell cycle genes in G1 at 4 months.

### Example 2-2: Inflammatory Response in EMPD

Transcriptomic data from the immune cell-rich region near the EMPD was analyzed, and the results using the CIBERSORTx algorithm were presented as a stacked bar plot (FIG. 2F). Relative and absolute cell fractions are provided in Supplementary Table S7 (Table 3). At 6 months of EMPD, an increase in CD8 T cells was observed, indicating an inflammatory signal. However, at one year, lymphoid cells such as resting memory CD4 T cells, Tregs, activated NK cells, and the like were recovered to baseline levels or slightly increased. Also, an increase in type 2 macrophages and neutrophils among myeloid cells was also observed (FIG. 2G).

TABLE 3

| | B cells | B cells | | | T cells CD4 | T cells CD4 |
|---|---|---|---|---|---|---|
| Mixture | naive | memory | Plasma cells | T cells CD8 | naive | memory |
| 0 month_{—} | 0 | 0.324804 | 0.164931 | 0.061683 | 0 | 0.584333 |
| Inflammation1 | | | | | | |
| 0 month_{—} | 0.30829 | 0 | 0.203791 | 0.150704 | 0.511354 | 0.267396 |
| Inflammation2 | | | | | | |
| 0 month_{—} | 0 | 0.554378 | 0.090767 | 0.064255 | 0.089916 | 0.295228 |
| Inflammation3 | | | | | | |
| 0 month_{—} | 0 | 0.324526 | 0.191815 | 0 | 0 | 0.287277 |
| Inflammation4 | | | | | | |
| 4 month_{—} | 0.033442 | 0.036749 | 0.002114 | 0 | 0.045559 | 0.2308 |
| Inflammation1 | | | | | | |
| 4 month_{—} | 0 | 0.240938 | 0.189115 | 0.124469 | 0.340262 | 0.155546 |
| Inflammation2 | | | | | | |
| 4 month_{—} | 0.145374 | 0 | 0.415262 | 0.093404 | 0 | 0.148791 |
| Inflammation3 | | | | | | |
| 4 month_{—} | 0 | 0.15664 | 0.259052 | 0.227588 | 0.006419 | 0 |
| Inflammation4 | | | | | | |
| 6 month_{—} | 0.150837 | 0.037918 | 0.002243 | 0.367175 | 0.400935 | 0 |
| Inflammation4 | | | | | | |
| 6 month_{—} | 0 | 0.305893 | 0.135197 | 0.442257 | 0 | 0.043837 |
| Inflammation1 | | | | | | |
| 6 month_{—} | 0.080726 | 0.017153 | 0.012021 | 0.212079 | 0.008773 | 0.533524 |
| Inflammation2 | | | | | | |
| 6 month_{—} | 0.030113 | 0.038095 | 0.018742 | 0.85738 | 0 | 0 |
| Inflammation3 | | | | | | |
| 1 year_{—} | 0 | 0.414023 | 0.163068 | 0.283116 | 0 | 0.121129 |
| Inflammation1 | | | | | | |
| 1 year_{—} | 0 | 0.197477 | 0.388474 | 0.17059 | 0 | 0.214422 |
| Inflammation2 | | | | | | |
| 1 year_{—} | 0 | 0.480573 | 0.023508 | 0.446793 | 0.017402 | 0.263571 |
| Inflammation3 | | | | | | |
| 1 year_{—} | 0.025621 | 0.154508 | 0.061471 | 0.311214 | 0 | 0.445662 |
| Inflammation4 | | | | | | |

| | T cells CD4 | T cells | T cells | T cells | NK cells |
|---|---|---|---|---|---|
| Mixture | memory | follicular | regulatory | gamma delta | resting |
| 0 month_{—} | 0 | 0 | 0.12641 | 0.071654 | 0 |
| Inflammation1 | | | | | |
| 0 month_{—} | 0.078301 | 0 | 0 | 0.058875 | 0.167308 |
| Inflammation2 | | | | | |
| 0 month_{—} | 0 | 0.064749 | 0.159431 | 0 | 0 |
| Inflammation3 | | | | | |
| 0 month_{—} | 0 | 0.199692 | 0.211717 | 0 | 0 |
| Inflammation4 | | | | | |
| 4 month_{—} | 0 | 0.000549 | 0.261345 | 0 | 0.433064 |
| Inflammation1 | | | | | |
| 4 month_{—} | 0.074232 | 0 | 0 | 0 | 0.38762 |
| Inflammation2 | | | | | |
| 4 month_{—} | 0.091241 | 0.126879 | 0.200348 | 0 | 0.092597 |
| Inflammation3 | | | | | |
| 4 month_{—} | 0.009517 | 0 | 0.098911 | 0 | 0.073847 |
| Inflammation4 | | | | | |
| 6 month_{—} | 0 | 0.210735 | 0.340157 | 0 | 0 |
| Inflammation4 | | | | | |
| 6 month_{—} | 0.138999 | 0.045803 | 0 | 0.248472 | 0 |
| Inflammation1 | | | | | |
| 6 month_{—} | 0.183971 | 0.030929 | 0 | 0 | 0.357882 |
| Inflammation2 | | | | | |
| 6 month_{—} | 0 | 0.201939 | 0.560859 | 0 | 0.047486 |
| Inflammation3 | | | | | |
| 1 year_{—} | 0 | 0.063301 | 0.391007 | 0.011551 | 0 |
| Inflammation1 | | | | | |
| 1 year_{—} | 0 | 0 | 0.287218 | 0 | 0 |
| Inflammation2 | | | | | |
| 1 year_{—} | 0 | 0.021318 | 0 | 0 | 0 |
| Inflammation3 | | | | | |
| 1 year_{—} | 0.001212 | 0.081481 | 0.312267 | 0 | 0 |
| Inflammation4 | | | | | |

| | | | | | | Dendritic |
|---|---|---|---|---|---|---|
| | NK cells | | Macrophages | Macrophages | Macrophages | cells |
| Mixture | activated | Monocytes | M0 | M1 | M2 | resting |
| 0 month_{—} | 0.264951 | 0.033701 | 0 | 0.038722 | 0.032796 | 0.041548 |
| Inflammation1 | | | | | | |
| 0 month_{—} | 0 | 0 | 0.013412 | 0.095119 | 0.128169 | 0 |
| Inflammation2 | | | | | | |
| 0 month_{—} | 0.195629 | 0.060695 | 0.068587 | 0.120597 | 0.070413 | 0.083357 |
| Inflammation3 | | | | | | |
| 0 month_{—} | 0.170421 | 0.056678 | 0 | 0.038259 | 0.063815 | 0 |
| Inflammation4 | | | | | | |
| 4 month_{—} | 0 | 0.095939 | 0 | 0.087122 | 0.046958 | 0.154949 |
| Inflammation1 | | | | | | |
| 4 month_{—} | 0 | 0.043942 | 0 | 0.019025 | 0.114327 | 0.02145 |
| Inflammation2 | | | | | | |
| 4 month_{—} | 0.009378 | 0.05174 | 0 | 0.022247 | 0.109007 | 0.06521 |
| Inflammation3 | | | | | | |
| 4 month_{—} | 0.018155 | 0 | 0.083649 | 0.07471 | 0.050803 | 0.047286 |
| Inflammation4 | | | | | | |
| 6 month_{—} | 0.391134 | 0 | 0.625977 | 0.700996 | 0.208371 | 0 |
| Inflammation4 | | | | | | |
| 6 month_{—} | 0 | 0 | 0 | 0.112019 | 0.184214 | 0.015149 |
| Inflammation1 | | | | | | |
| 6 month_{—} | 0 | 0 | 0.028833 | 0.115322 | 0.095857 | 0.064012 |
| Inflammation2 | | | | | | |
| 6 month_{—} | 0.19827 | 0.025355 | 0.056295 | 0.16807 | 0.113836 | 0.141757 |
| Inflammation3 | | | | | | |
| 1 year_{—} | 0.269998 | 0.041634 | 0 | 0.080913 | 0.295775 | 0.08847 |
| Inflammation1 | | | | | | |
| 1 year_{—} | 0.252342 | 0.080014 | 0 | 0.039145 | 0.141608 | 0.049931 |
| Inflammation2 | | | | | | |
| 1 year_{—} | 0.212349 | 0.058105 | 0 | 0.024881 | 0.050113 | 0.04673 |
| Inflammation3 | | | | | | |
| 1 year_{—} | 0.135224 | 0.043035 | 0.006191 | 0.022475 | 0.079242 | 0.010844 |
| Inflammation4 | | | | | | |

| | | Mast | | | |
|---|---|---|---|---|---|
| | Dendritic | cells | Mast cells | | |
| Mixture | cells | resting | activated | Eosinophils | Neutrophils |
| 0 month_{—} | 0.048034 | 0.59584 | 0 | 0 | 0 |
| Inflammation1 | | | | | |
| 0 month_{—} | 0.050474 | 0.740656 | 0 | 0 | 0.053728 |
| Inflammation2 | | | | | |
| 0 month_{—} | 0.004695 | 0.345754 | 0.003337 | 0 | 0 |
| Inflammation3 | | | | | |
| 0 month_{—} | 0 | 0 | 0.705699 | 0.085736 | 0 |
| Inflammation4 | | | | | |
| 4 month_{—} | 0 | 0.079234 | 0.359588 | 0.077492 | 0 |
| Inflammation1 | | | | | |
| 4 month_{—} | 0 | 0.048306 | 0.074425 | 0.065608 | 0.014643 |
| Inflammation2 | | | | | |
| 4 month_{—} | 0 | 0.457802 | 0.069522 | 0 | 0 |
| Inflammation3 | | | | | |
| 4 month_{—} | 0 | 0.812892 | 0.024123 | 0 | 0.010993 |
| Inflammation4 | | | | | |
| 6 month_{—} | 0 | 0.591176 | 0 | 0 | 0 |
| Inflammation4 | | | | | |
| 6 month_{—} | 0 | 0.096952 | 0 | 0.280546 | 0 |
| Inflammation1 | | | | | |
| 6 month_{—} | 0.003848 | 0.108293 | 0.143612 | 0.035435 | 0.01097 |
| Inflammation2 | | | | | |
| 6 month_{—} | 0 | 0.09185 | 0.027909 | 0 | 0.017112 |
| Inflammation3 | | | | | |
| 1 year_{—} | 0 | 0.154001 | 0 | 0 | 0.043048 |
| Inflammation1 | | | | | |
| 1 year_{—} | 0 | 0.49925 | 0 | 0 | 0.044381 |
| Inflammation2 | | | | | |
| 1 year_{—} | 0.05906 | 0.05805 | 0.059538 | 0.045046 | 0.0635 |
| Inflammation3 | | | | | |
| 1 year_{—} | 0.021964 | 0.313461 | 0.052928 | 0.006709 | 0.014606 |
| Inflammation4 | | | | | |

| | | | | | | T cells CD4 |
|---|---|---|---|---|---|---|
| | B cells | B cells | | | T cells CD4 | memory |
| Mixture | naive | memory | Plasma cells | T cells CD8 | naive | resting |
| 0 month_{—} | 0 | 0.135935 | 0.069026 | 0.025815 | 0 | 0.244551 |
| Inflammation1 | | | | | | |
| 0 month_{—} | 0.10903 | 0 | 0.072073 | 0.053298 | 0.180845 | 0.094567 |
| Inflammation2 | | | | | | |
| 0 month_{—} | 0 | 0.244027 | 0.039954 | 0.028284 | 0.03958 | 0.129954 |
| Inflammation3 | | | | | | |
| 0 month_{—} | 0 | 0.138946 | 0.082125 | 0 | 0 | 0.122997 |
| Inflammation4 | | | | | | |
| 4 month_{—} | 0.017195 | 0.018895 | 0.001087 | 0 | 0.023425 | 0.118669 |
| Inflammation1 | | | | | | |
| 4 month_{—} | 0 | 0.125888 | 0.098811 | 0.065034 | 0.177784 | 0.081271 |
| Inflammation2 | | | | | | |
| 4 month_{—} | 0.069265 | 0 | 0.197857 | 0.044504 | 0 | 0.070893 |
| Inflammation3 | | | | | | |
| 4 month_{—} | 0 | 0.08014 | 0.132535 | 0.116438 | 0.003284 | 0 |
| Inflammation4 | | | | | | |
| 6 month_{—} | 0.03745 | 0.009414 | 0.000557 | 0.091163 | 0.099545 | 0 |
| Inflammation4 | | | | | | |
| 6 month_{—} | 0 | 0.149264 | 0.065971 | 0.215805 | 0 | 0.021391 |
| Inflammation1 | | | | | | |
| 6 month_{—} | 0.039509 | 0.008395 | 0.005883 | 0.103796 | 0.004294 | 0.261116 |
| Inflammation2 | | | | | | |
| 6 month_{—} | 0.011604 | 0.01468 | 0.007222 | 0.330388 | 0 | 0 |
| Inflammation3 | | | | | | |
| 1 year_{—} | 0 | 0.171011 | 0.067355 | 0.11694 | 0 | 0.050032 |
| Inflammation1 | | | | | | |
| 1 year_{—} | 0 | 0.083505 | 0.16427 | 0.072135 | 0 | 0.09067 |
| Inflammation2 | | | | | | |
| 1 year_{—} | 0 | 0.248932 | 0.012177 | 0.231435 | 0.009014 | 0.136527 |
| Inflammation3 | | | | | | |
| 1 year_{—} | 0.0122 | 0.073571 | 0.029271 | 0.148189 | 0 | 0.212209 |
| Inflammation4 | | | | | | |

| | T cells CD4 | T cells | T cells | | |
|---|---|---|---|---|---|
| | memory | follicular | regulatory | T cells | NK cells |
| Mixture | activated | helper | (Tregs) | gamma delta | resting |
| 0 month_{—} | 0 | 0 | 0.052904 | 0.029988 | 0 |
| Inflammation1 | | | | | |
| 0 month_{—} | 0.027692 | 0 | 0 | 0.020822 | 0.05917 |
| Inflammation2 | | | | | |
| 0 month_{—} | 0 | 0.028501 | 0.070179 | 0 | 0 |
| Inflammation3 | | | | | |
| 0 month_{—} | 0 | 0.085498 | 0.090646 | 0 | 0 |
| Inflammation4 | | | | | |
| 4 month_{—} | 0 | 0.000282 | 0.134374 | 0 | 0.222666 |
| Inflammation1 | | | | | |
| 4 month_{—} | 0.038786 | 0 | 0 | 0 | 0.202528 |
| Inflammation2 | | | | | |
| 4 month_{—} | 0.043473 | 0.060453 | 0.095458 | 0 | 0.044119 |
| Inflammation3 | | | | | |
| 4 month_{—} | 0.004869 | 0 | 0.050605 | 0 | 0.037781 |
| Inflammation4 | | | | | |
| 6 month_{—} | 0 | 0.052322 | 0.084455 | 0 | 0 |
| Inflammation4 | | | | | |
| 6 month_{—} | 0.067826 | 0.02235 | 0 | 0.121245 | 0 |
| Inflammation1 | | | | | |
| 6 month_{—} | 0.090039 | 0.015137 | 0 | 0 | 0.175154 |
| Inflammation2 | | | | | |
| 6 month_{—} | 0 | 0.077816 | 0.216125 | 0 | 0.018298 |
| Inflammation3 | | | | | |
| 1 year_{—} | 0 | 0.026146 | 0.161504 | 0.004771 | 0 |
| Inflammation1 | | | | | |
| 1 year_{—} | 0 | 0 | 0.121453 | 0 | 0 |
| Inflammation2 | | | | | |
| 1 year_{—} | 0 | 0.011042 | 0 | 0 | 0 |
| Inflammation3 | | | | | |
| 1 year_{—} | 0.000577 | 0.038798 | 0.148691 | 0 | 0 |
| Inflammation4 | | | | | |

| | | | | | | Dendritic |
|---|---|---|---|---|---|---|
| | NK cells | | Macrophage | Macrophages | Macrophages | cells |
| Mixture | activated | Monocytes | M0 | M1 | M2 | resting |
| 0 month_{—} | 0.110886 | 0.014104 | 0 | 0.016206 | 0.013726 | 0.017388 |
| Inflammation | | | | | | |
| 0 month_{—} | 0 | 0 | 0.004743 | 0.03364 | 0.045328 | 0 |
| Inflammation2 | | | | | | |
| 0 month_{—} | 0.086112 | 0.026717 | 0.030191 | 0.053085 | 0.030994 | 0.036692 |
| Inflammation3 | | | | | | |
| 0 month_{—} | 0.072966 | 0.024267 | 0 | 0.01638 | 0.027322 | 0 |
| Inflammation4 | | | | | | |
| 4 month_{—} | 0 | 0.049328 | 0 | 0.044795 | 0.024144 | 0.079669 |
| Inflammation1 | | | | | | |
| 4 month_{—} | 0 | 0.02296 | 0 | 0.00994 | 0.059735 | 0.011207 |
| Inflammation2 | | | | | | |
| 4 month_{—} | 0.004468 | 0.024652 | 0 | 0.0106 | 0.051938 | 0.03107 |
| Inflammation3 | | | | | | |
| 4 month_{—} | 0.009288 | 0 | 0.042796 | 0.038223 | 0.025991 | 0.024192 |
| Inflammation4 | | | | | | |
| 6 month_{—} | 0.097112 | 0 | 0.15542 | 0.174046 | 0.051735 | 0 |
| Inflammation4 | | | | | | |
| 6 month_{—} | 0 | 0 | 0 | 0.054661 | 0.089889 | 0.007392 |
| Inflammation1 | | | | | | |
| 6 month_{—} | 0 | 0 | 0.014111 | 0.056441 | 0.046914 | 0.031329 |
| Inflammation2 | | | | | | |
| 6 month_{—} | 0.076403 | 0.00977 | 0.021693 | 0.064765 | 0.043866 | 0.054626 |
| Inflammation3 | | | | | | |
| 1 year_{—} | 0.111522 | 0.017197 | 0 | 0.033421 | 0.122169 | 0.036542 |
| Inflammation1 | | | | | | |
| 1 year_{—} | 0.106705 | 0.033834 | 0 | 0.016553 | 0.05988 | 0.021114 |
| Inflammation2 | | | | | | |
| 1 year_{—} | 0.109995 | 0.030098 | 0 | 0.012888 | 0.025958 | 0.024206 |
| Inflammation3 | | | | | | |
| 1 year_{—} | 0.064389 | 0.020492 | 0.002948 | 0.010702 | 0.037732 | 0.005163 |
| Inflammation4 | | | | | | |

| | Dendritic | Mast | | | |
|---|---|---|---|---|---|
| | cells | cells | Mast cells | | |
| Mixture | activated | resting | activated | Eosinophils | Neutrophils |
| 0 month_{—} | 0.020103 | 0.24937 | 0 | 0 | 0 |
| Inflammation] | | | | | |
| 0 month_{—} | 0.01785 | 0.26194 | 0 | 0 | 0.019002 |
| Inflammation2 | | | | | |
| 0 month_{—} | 0.002067 | 0.152195 | 0.001469 | 0 | 0 |
| Inflammation3 | | | | | |
| 0 month_{—} | 0 | 0 | 0.302144 | 0.036708 | 0 |
| Inflammation4 | | | | | |
| 4 month_{—} | 0 | 0.040739 | 0.184887 | 0.039843 | 0 |
| Inflammation1 | | | | | |
| 4 month_{—} | 0 | 0.025239 | 0.038886 | 0.03428 | 0.007651 |
| Inflammation2 | | | | | |
| 4 month_{—} | 0 | 0.218125 | 0.033125 | 0 | 0 |
| Inflammation3 | | | | | |
| 4 month_{—} | 0 | 0.41589 | 0.012342 | 0 | 0.005624 |
| Inflammation4 | | | | | |
| 6 month_{—} | 0 | 0.146779 | 0 | 0 | 0 |
| Inflammation4 | | | | | |
| 6 month_{—} | 0 | 0.047301 | 0 | 0.136896 | 0 |
| Inflammation1 | | | | | |
| 6 month_{—} | 0.001883 | 0.053001 | 0.070287 | 0.017343 | 0.005369 |
| Inflammation2 | | | | | |
| 6 month_{—} | 0 | 0.035394 | 0.010755 | 0 | 0.006594 |
| Inflammation3 | | | | | |
| 1 year_{—} | 0 | 0.06361 | 0 | 0 | 0.017781 |
| Inflammation1 | | | | | |
| 1 year_{—} | 0 | 0.211112 | 0 | 0 | 0.018767 |
| Inflammation2 | | | | | |
| 1 year_{—} | 0.030593 | 0.030069 | 0.03084 | 0.023333 | 0.032893 |
| Inflammation3 | | | | | |
| 1 year_{—} | 0.010458 | 0.149259 | 0.025202 | 0.003195 | 0.006955 |
| Inflammation4 | | | | | |

Also, given the increased levels of PD-1 reported in EMPD cases, examining PD-1 and PD-L1 expression is key to understanding responses to immune checkpoint therapy. Data showed consistent PD-1 (PDCD1) expression at baseline. 4 months, 6 months, and one year. In contrast, PD-L1 (CD274) expression significantly increased at 6 months and decreased at one year, deviating from the observed normal trend (FIG. 2H). Exhausted CD8+ T cell markers HAVCR2, GZMB, TNF, and CTLA4 decreased at 4 and 6 months, but increased after one year.

Based on the results of analysis over time, it was confirmed that the expression of MHC group 1-related genes, especially HLA genes of MHC group 1, was abnormally low at 4 months (FIG. 2I). MHC group 1 molecules are essential for presenting peptide presentation to CD8+ T cells, and their low expression on tumor cells may hinder immune recognition and elimination, thereby allowing tumor evasion.

### Example 3: Hyperactivation of mTOR Pathway and Identification of SPDEF Markers in EMPD

Through this example, EMPD characteristics were defined through comparative analysis of baseline EMPD, normal, and invasive EMPD groups.

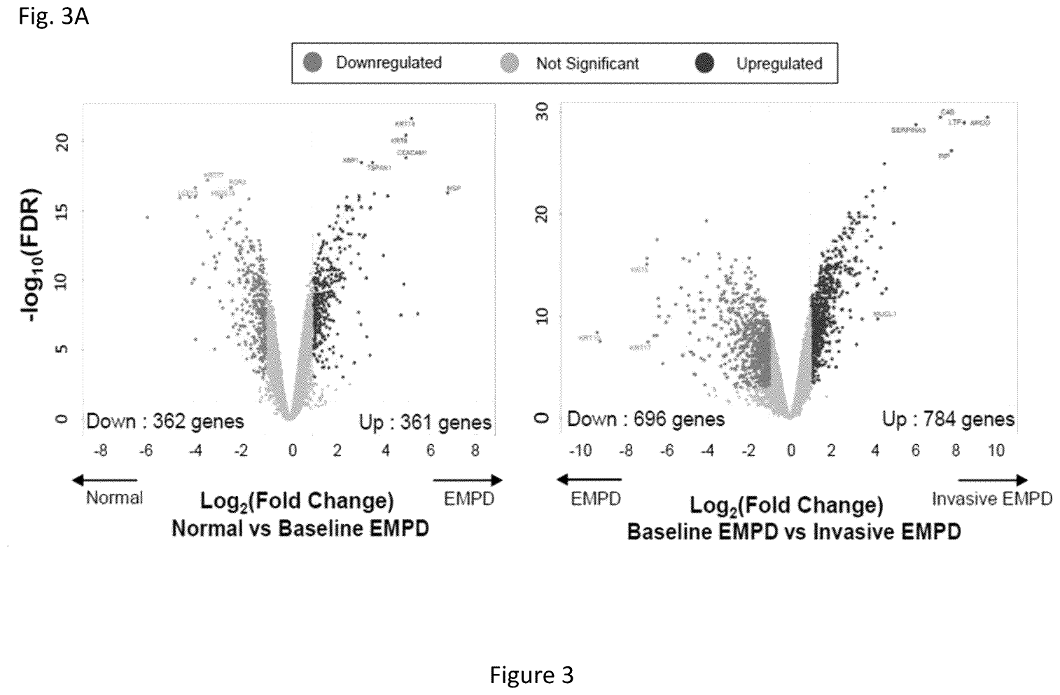

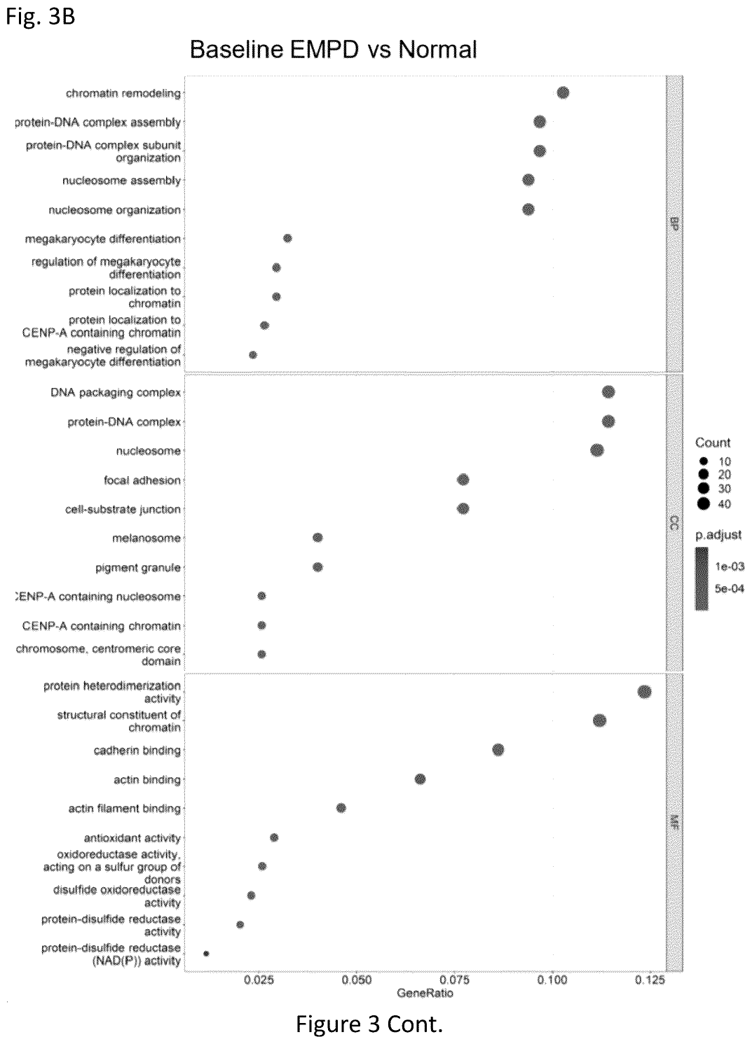

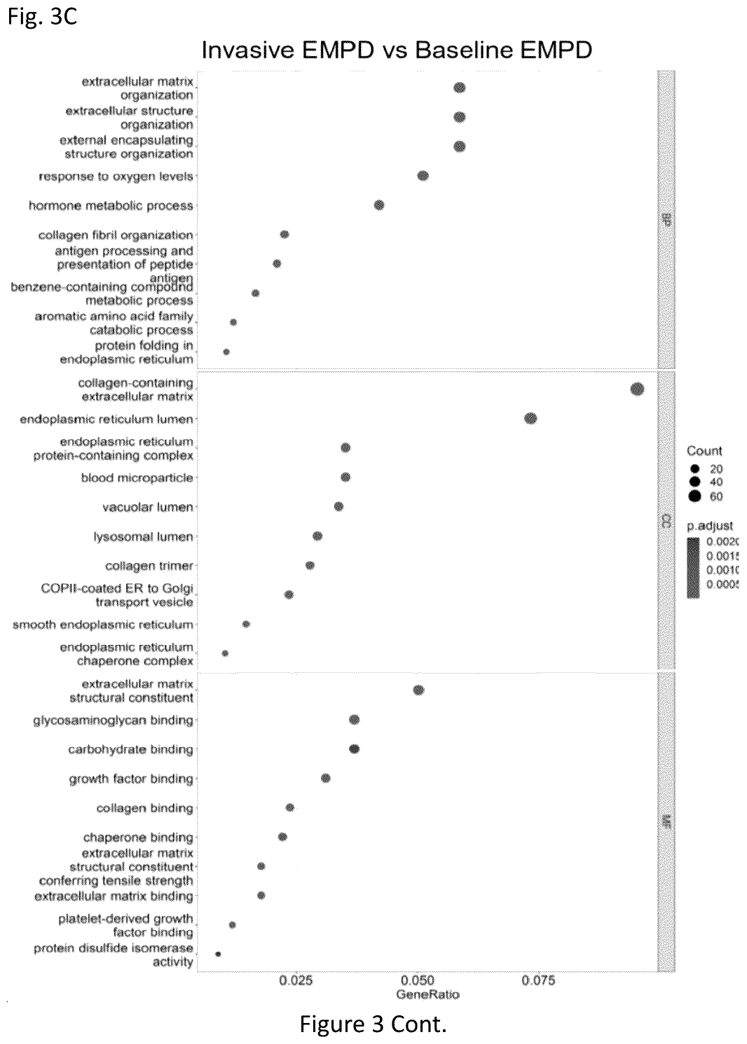

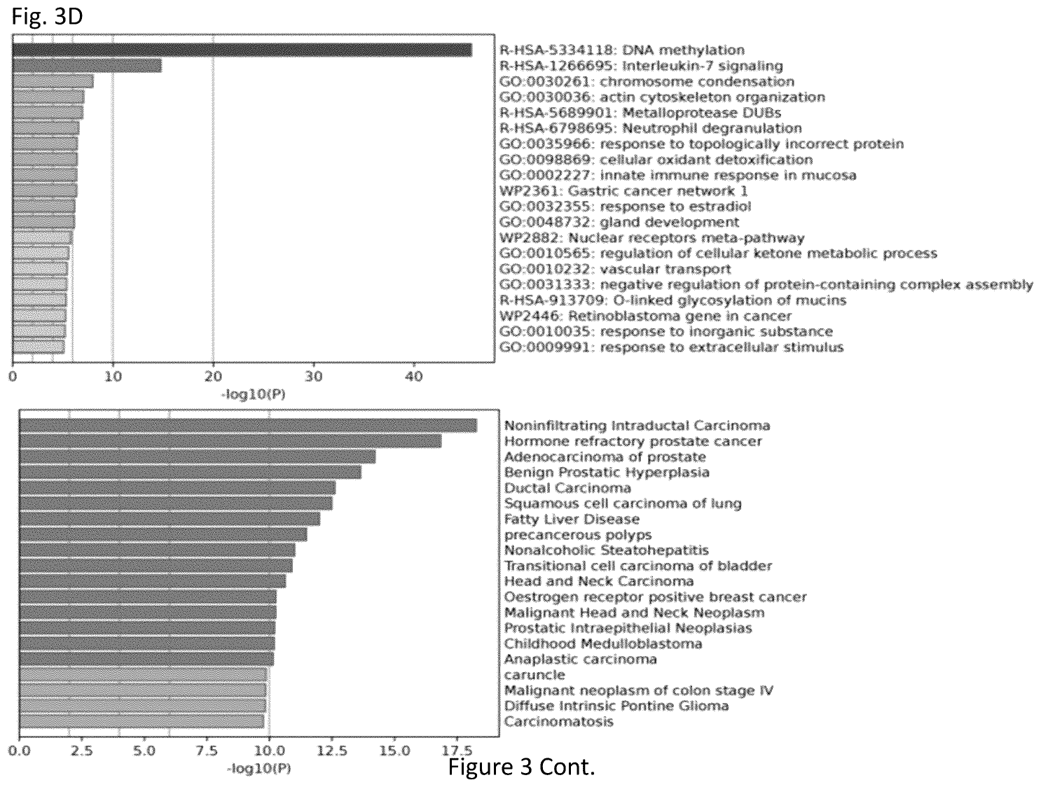

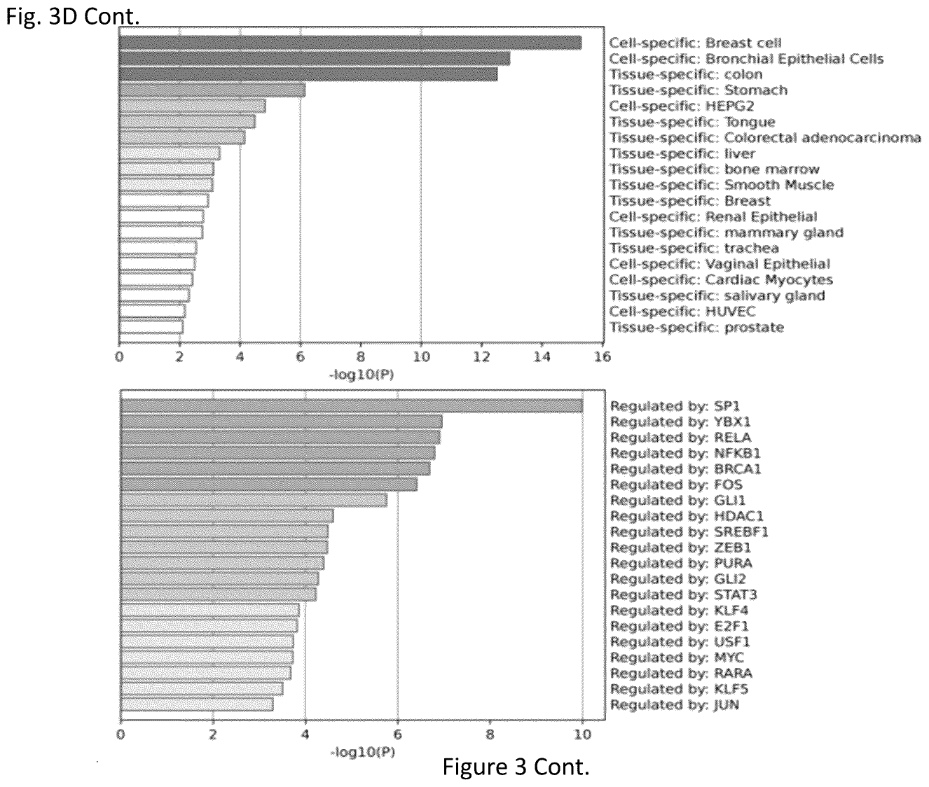

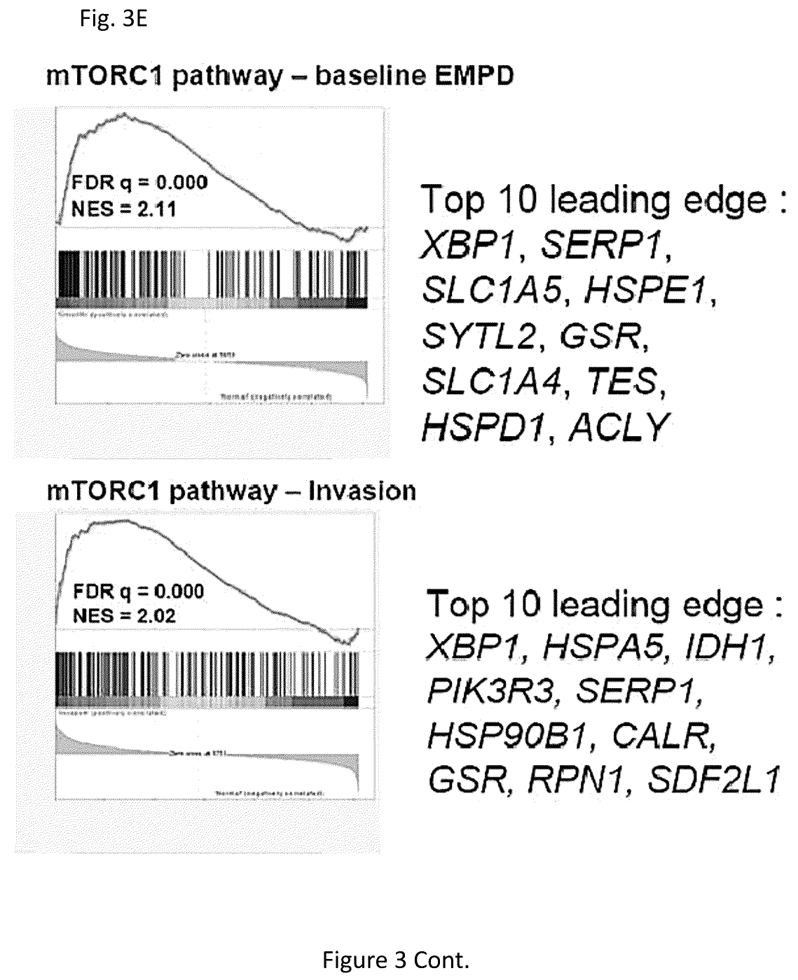

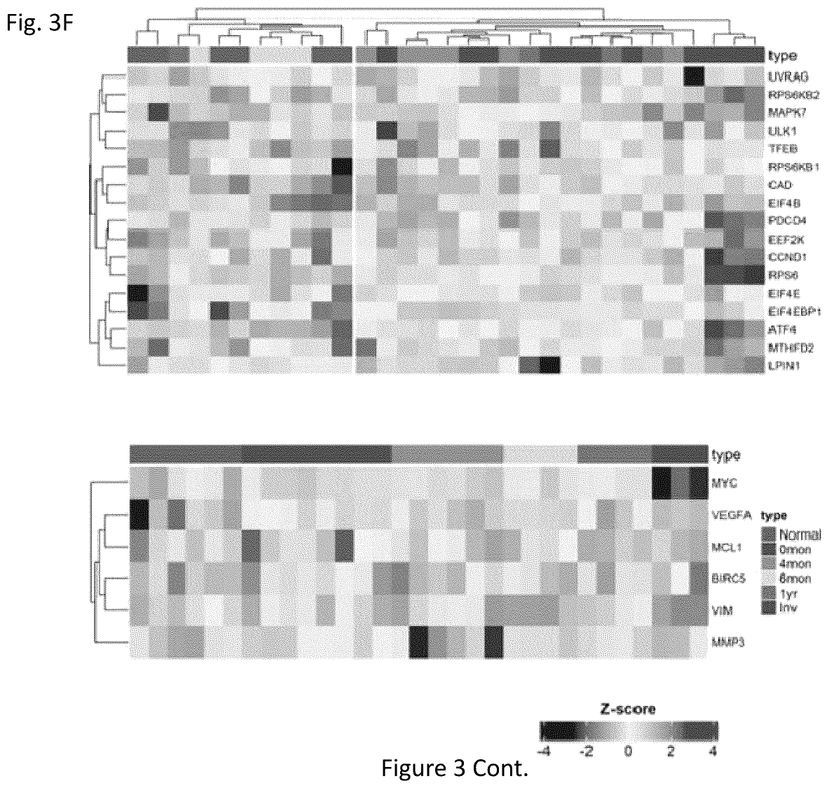

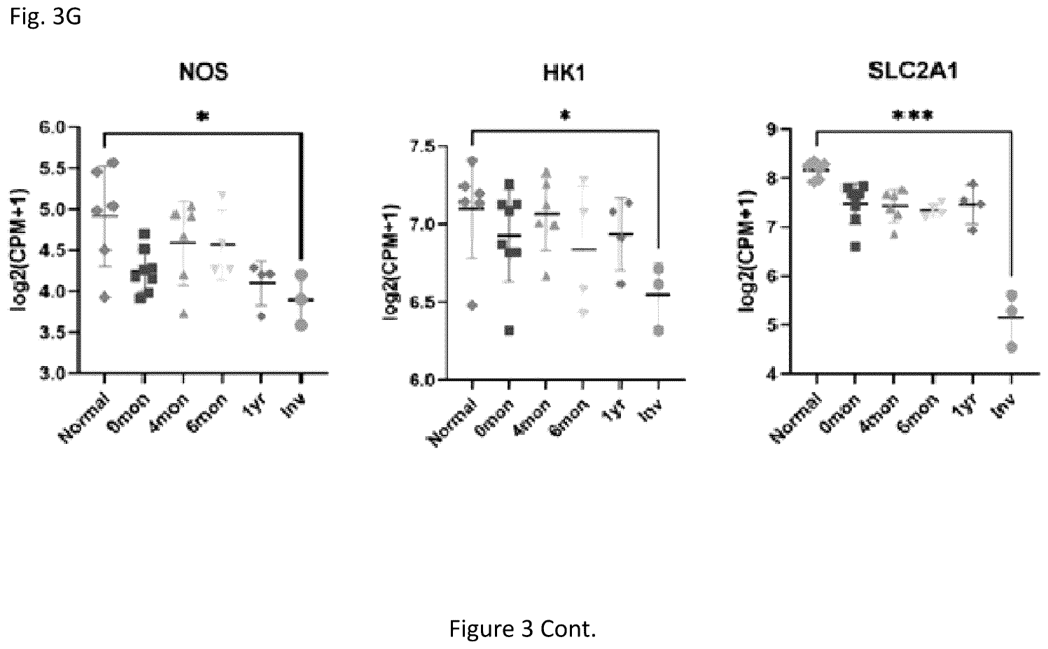

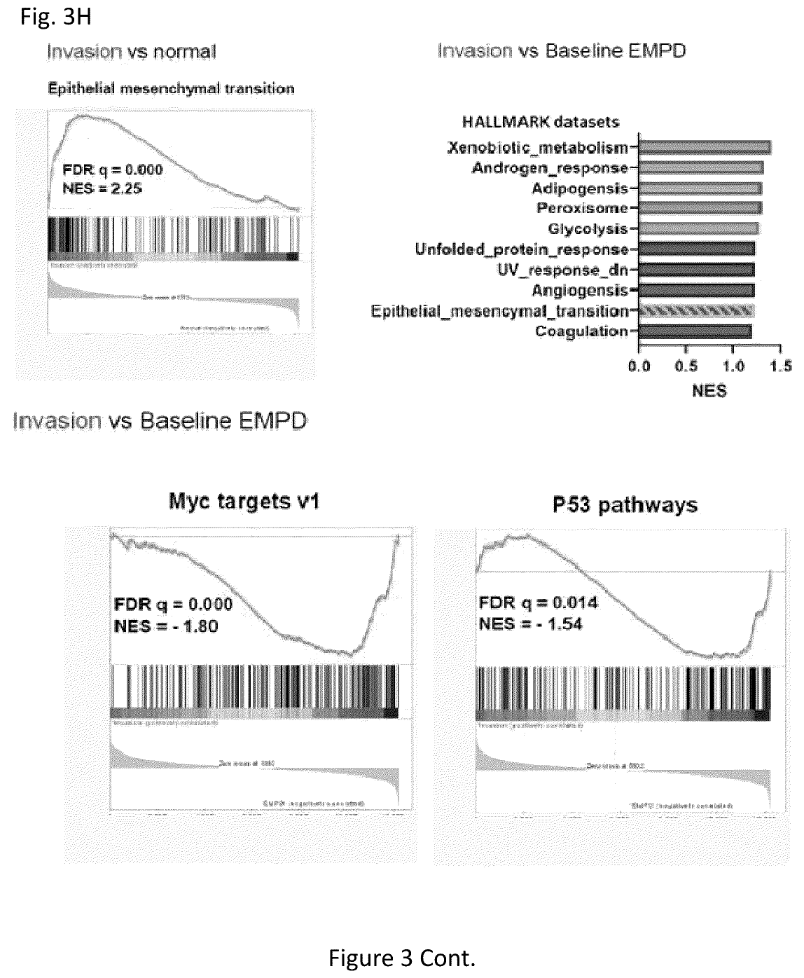

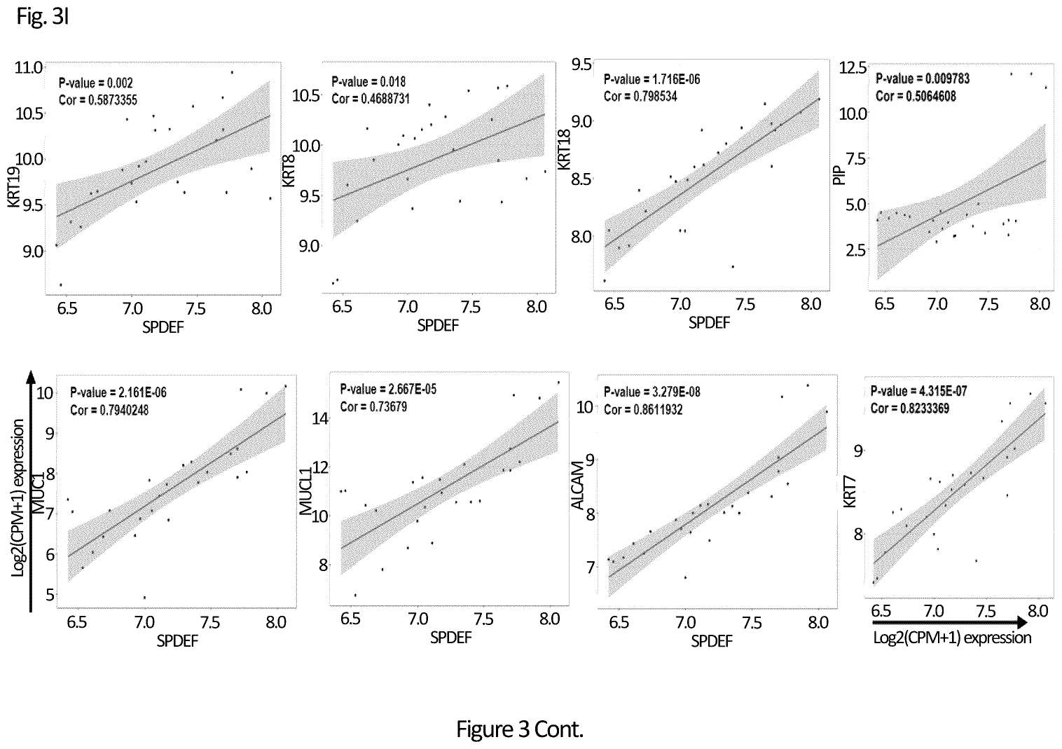

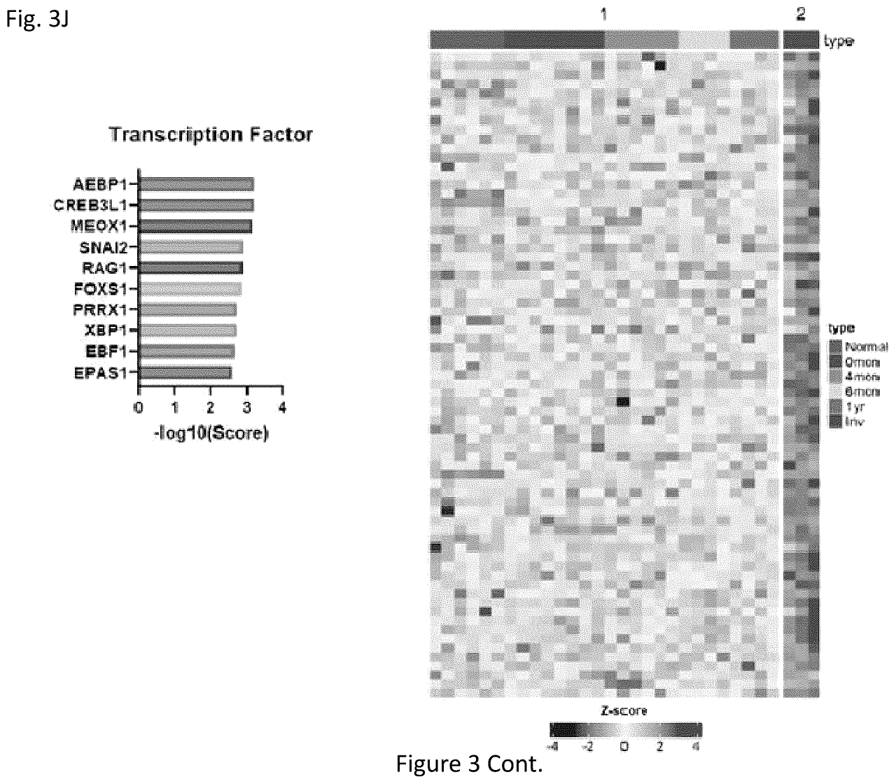

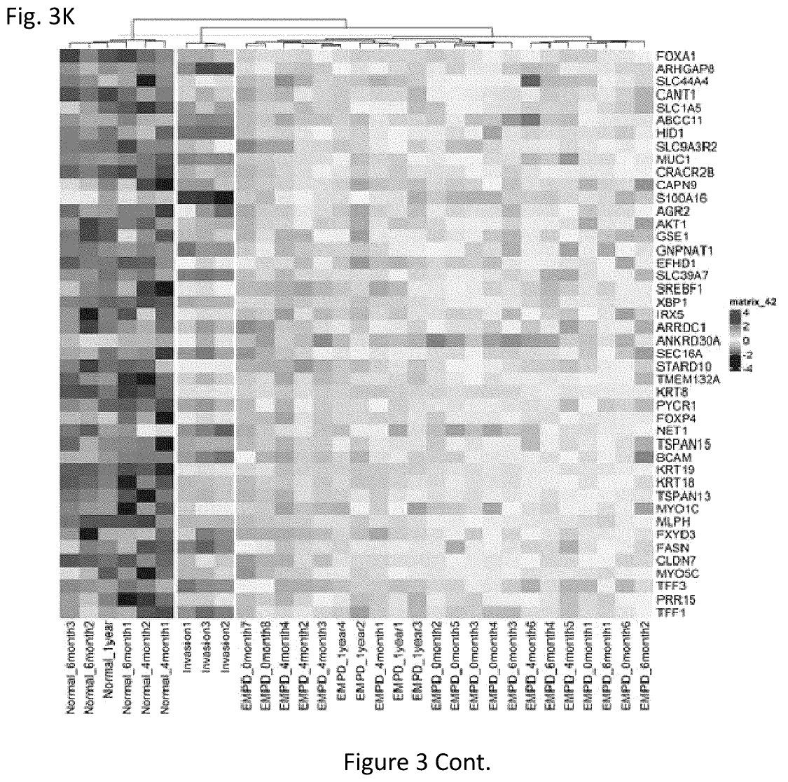

First, 723 DEGs were identified through the comparison between the normal and baseline EMPD samples. As a result, 361 of the 723 DEGs were up-regulated and 362 were down-regulated in baseline EMPD. The up-regulated genes were identified to include known Paget biomarkers including SPDEF. Also, the invasive EMPD samples showed 1,480 DEGs compared to baseline EMPD. Among them, 784 DEGs were up-regulated and 696 DEGs were down-regulated. SPDEF and AEBP1 were included in the up-regulated genes. In addition, genes linked to sweat gland cells, such as PIP, APOD, and MUCL1, were included, showing low p-values and high log₂ fold changes (FIG. 3A).

Also, GO analysis and enrichment analysis were performed on both baseline EMPD and invasive EMPD samples. In the baseline EMPD sample, low p values were observed for biological processes (BP), cellular components (CC), and molecular functions (MF) related to chromosomes, DNA, and nucleosome assembly and organization (FIG. 3B). Compared to baseline, the invasive EMPD samples showed the up-regulation of genes related to the collagen-containing extracellular matrix and endoplasmic reticulum processes, with low p-values in oxygen level-related pathways (FIG. 3C).

Based on the GO analysis, it was confirmed that the genes included in the reactive oxygen species (ROS)-related pathway (that is, a hypoxia-related pathway) and the epidermal mesenchymal transition-related pathway increased in both the baseline EMPD and invasive EMPD samples. This finding has not been previously reported.

From the functional enrichment analysis of EMPD using the Reactome database, pathways including myc, oxidative phosphorylation, cell cycle, and DNA processing were identified (Table 4). Also, to distinguish the normal EMPD samples from the entire EMPD samples using Metascape, the related GO terms and pathways were analyzed to confirm their association with DNA- and chromatin-related processes (FIG. 3D).

TABLE 4
Baseline EMPD VS Normal

| | Gene Set Details | NES | FDR Q-value |
|---|---|---|---|
| 1 | MYC targets V1 | 3.35 | 0 |
| 2 | Oxidative Phosphorylation | 3.03 | 0 |
| 3 | E2F targets | 2.85 | 0 |
| 4 | MYC targets V2 | 2.57 | 0 |
| 5 | G2M checkpoint | 2.57 | 0 |
| 6 | Unfolded Protein Response | 2.34 | 0 |
| 7 | DNA Repair | 2.28 | 0 |
| 8 | Mtorc1 signaling | 2.11 | 0 |
| 9 | Mitotic Spindle | 2.06 | 0 |
| 10 | Androgen Response | 1.92 | 0 |
| 11 | Reactive Oxygen Species Pathway | 1.92 | 0 |
| 12 | Adipogenesis | 1.88 | 0.001 |
| 13 | Glycolysis | 1.76 | 0.003 |
| 14 | Apoptosis | 1.71 | 0.004 |
| 15 | Protein Secretion | 1.7 | 0.004 |
| 16 | Fatty Acid Metabolism | 1.59 | 0.013 |
| 17 | UV Response Up | 1.53 | 0.022 |
| 18 | Cholestoerol Homeostasis | 1.49 | 0.028 |
| 19 | TGF Beta signaling | 1.46 | 0.035 |
| 20 | Xenobiotic Metabolism | 1.46 | 0.035 |

As a result, the mTORC1 signaling pathway had a high normalized enrichment score (NES) of 2.11 in baseline EMPD, indicating significant enrichment (FDR=0.000) (FIG. 3E). No significant pathways were found when comparing invasive EMPD to baseline EMPD (Table 5), but invasive EMPD also showed a higher mTORC1 pathway NES value (NES=2.02, FDR=0.000) when compared to normal.

TABLE 5
Invasion vs Baseline EMPD

| | Gene Set Details | NES | FDR Q-value |
|---|---|---|---|
| 1 | Xenobiotic Metabolism | 1.4 | 1 |
| 2 | Androgen Response | 1.32 | 1 |
| 3 | Adipogenesis | 1.3 | 0.904 |
| 4 | Peroxisome | 1.3 | 0.719 |
| 5 | Glycolysis | 1.28 | 0.653 |
| 6 | Unfolded Protein Response | 1.24 | 0.689 |
| 7 | EV response DN | 1.23 | 0.609 |
| 8 | Angiogenesis | 1.23 | 0.537 |
| 9 | Epithelial Mesenchymal Transition | 1.23 | 0.496 |
| 10 | Coagulation | 1.2 | 0.511 |
| 11 | Protein Secretion | 1.15 | 0.611 |
| 12 | Mtroc1 Signaling | 1.13 | 0.633 |
| 13 | Hedgehog Signaling | 1.13 | 0.59 |
| 14 | Kras Signaling UP | 1.12 | 0.557 |
| 15 | Myogenesis | 1.12 | 0.53 |
| 16 | Fatty Acid Metabolism | 1.11 | 0.523 |
| 17 | Bile Acid Metabolism | 1.1 | 0.499 |
| 18 | Complement | 1.07 | 0.525 |
| 19 | Reactive Oxygen Species Pathway | 1.01 | 0.617 |
| 20 | IL2 Stat5 Signaling | 0.99 | 0.621 |

Also, the present inventors examined whether the genes related to the mTORC1 pathway were up-regulated. As a result, it was found that the 6-month pattern of EMPD was very similar to that of the normal samples. Invasive EMPD showed distinct and unique patterns of up- and down-regulated genes (FIG. 3F). Invasive EMPD showed uniquely increased HIF1α expression, and the associated downstream genes SLC2A1, PDK1, PFKFB3, PFKM, PFKP, and ALDOC had significantly lower values than normal samples. This suggests a potential role for the loss of oxygen signaling (FIG. 3G).

In addition, to evaluate epithelial-mesenchymal transition (EMT) associations including VEGFA, MCL1, BIRC5, MMP3, and VIM, mTORC1-sensitive mRNA was investigated in high EMT invasive EMPD.

As a result, the initially high MYC expression in baseline EMPD decreased in invasive EMPD (FIG. 3F). GSEA analysis showed significant up-regulation of EMT and reduction of myc targets and p53 pathways in the combined dataset of EMPD and invasive EMPD over time (FIG. 3H).

ChEA3 was used to identify multiomics data used to predict transcription factors. ChEA3 uses various library references (ENCODE, chip data, enricher data, GTEx) to obtain transcription factors. At this time, top rank refers to the highest rank among all these data, and mean rank refers to the average of all these data. It means the value when calculated as a value.

As a result, it was confirmed that SPDEF was the highest-priority gene in baseline EMPD and was regulated by FOXA1 (Table 6). FOXA1 is one of the known components of EMPD at proteomic and transcriptomic levels. According to the correlation graph, it was confirmed that SPDEF is an EMPD biomarker and has a positive correlation with other EMPD markers (FIG. 3I, Table 7). That is, SPDEF was confirmed to be an up-regulated biomarker in baseline EMPD. In the analysis of transcription factors, the unique up-expression pattern of AEBP1 expression distinguished the invasive EMPD group from the EMPD group (FIG. 3J), highlighting the unique characteristics of the EMPD and invasive EMPD groups despite the shared hyperactivation of the mTORC1 pathway.

TABLE 6

| | | Transcription | Integrated | Overlapping | |
|---|---|---|---|---|---|
| — | Rank | Factor | Scaled Rank | Genes | Library |
| Top | 1 | SPDEF | 6.14E−04 | 46 | ARCHS4 Coexpression |
| Rank | 2 | MECOM | 6.22E−04 | 41 | GTEx Coexpression |
| | 3 | CREB3L4 | 7.12E−04 | 50 | Enrichr Queries |
| | 4 | XBP1 | 0.001229 | 46 | ARCHS4 Coexpression |
| | 5 | CREB3L1 | 0.001245 | 41 | GTEx Coexpression |
| | 6 | FOXA1 | 0.001867 | 39 | GTEx Coexpression |
| | 7 | MYC | 0.002137 | 44 | Enrichr Queries |
| | 8 | ASCL3 | 0.002489 | 38 | GTEx Coexpression |
| | 9 | HMGA1 | 0.002849 | 44 | Enrichr Queries |
| | 10 | GRHL2 | 0.003071 | 36 | ARCHS4 Coexpression |

| | | Transcription | Mean | Overlapping | |
|---|---|---|---|---|---|
| — | Rank | Factor | Rank | Genes | Library |
| Mean | 1 | SPDEF | 3.333 | 94 | ARCHS4 Coexpression, 1; |
| Rank | | | | | Enrichr Queries, 2; |
| | | | | | GTEx Coexpression, 7 |
| | 2 | CREB3L4 | 5.667 | 89 | ARCHS4 Coexpression, 3; |
| | | | | | Enrichr Queries, 1; |
| | | | | | GTEx Coexpression, 13 |
| | 3 | ELF3 | 6.75 | 106 | ARCHS4 Coexpression, 7; |
| | | | | | Enrichr Queries, 6; |
| | | | | | ReMap ChIP-seq, 8; |
| | | | | | GTEx Coexpression, 6 |
| | 4 | FOXA1 | 22.83 | 149 | Literatrue ChIP-seq, 65; |
| | | | | | ARCHS4 Coexpression, 4; |
| | | | | | ENCODE ChIP-seq, 45; |
| | | | | | Enrichr Queries, 9; |
| | | | | | ReMap ChIP-seq, 11; |
| | | | | | GTEx Coexpression, 3 |
| | 5 | FOSL1 | 23.6 | 95 | ARCHS4 Coexpreesion, 15; |
| | | | | | ENCODE ChIP-seq, 16; |
| | | | | | Enrichr Queries, 18; |
| | | | | | ReMap ChIP-seq, 38; |
| | | | | | GTEx Coexpression, 31 |
| | 6 | CDX1 | 33.67 | 49 | ARCHS4 Coexpression, 34; |
| | | | | | Enrichr Queries, 49; |
| | | | | | GTEx Coexpression, 18 |
| | 7 | ZBTB42 | 36.5 | 40 | ARCHS4 Coexpression, 12; |
| | | | | | GTEx Coexpression, 61 |
| | 8 | ELF5 | 38.6 | 97 | Literature ChIP-seq, 40; |
| | | | | | ARCHS4 Coexpression, 36; |
| | | | | | Enrichr Queries, 21; |
| | | | | | ReMap ChIP-seq, 91; |
| | | | | | GTEx Coexpression, 5 |
| | 9 | CENPA | 48 | 22 | ARCHS4 Coexpression, 58; |
| | | | | | GTEx Coexpression, 38 |
| | 10 | ARNTL2 | 50.33 | 60 | ARCHS4 Coexpression, 17; |
| | | | | | Enrichr Queries, 38; |
| | | | | | GTEx Coexpression, 96 |

In addition, heatmap analysis was performed to confirm the relationship between SPDEF, baseline EMPD, and invasive EMPD. As a result, according to the heatmap in FIG. 3K, the expression of genes related to SPDEF was found at a high level in both EMPD and invasive EMPD, confirming that SPDEF is a major marker of EMPD and invasive EMPD.

In addition, according to FIG. 3I, when the correlation between most of the markers seen in EMPD and SPDEF was confirmed, they were all observed to be correlated, proving once again that SPDEF is a factor associated with EMPD.

TABLE 7

| | B cells | B cells | | | T cells CD4 | T cells CD4 |
|---|---|---|---|---|---|---|
| Mixture | naive | memory | Plasma cells | T cells CD8 | naive | memory |
| 0 month_{—} | 0 | 0.324804 | 0.164931 | 0.061683 | 0 | 0.584333 |
| Inflammation1 | | | | | | |
| 0 month_{—} | 0.30829 | 0 | 0.203791 | 0.150704 | 0.511354 | 0.267396 |
| Inflammation2 | | | | | | |
| 0 month_{—} | 0 | 0.554378 | 0.090767 | 0.064255 | 0.089916 | 0.295228 |
| Inflammation3 | | | | | | |
| 0 month_{—} | 0 | 0.324526 | 0.191815 | 0 | 0 | 0.287277 |
| Inflammation4 | | | | | | |
| 4 month_{—} | 0.033442 | 0.036749 | 0.002114 | 0 | 0.045559 | 0.2308 |
| Inflammation1 | | | | | | |
| 4 month_{—} | 0 | 0.240938 | 0.189115 | 0.124469 | 0.340262 | 0.155546 |
| Inflammation2 | | | | | | |
| 4 month_{—} | 0.145374 | 0 | 0.415262 | 0.093404 | 0 | 0.148791 |
| Inflammation3 | | | | | | |
| 4 month_{—} | 0 | 0.15664 | 0.259052 | 0.227588 | 0.006419 | 0 |
| Inflammation4 | | | | | | |
| 6 month_{—} | 0.150837 | 0.037918 | 0.002243 | 0.367175 | 0.400935 | 0 |
| Inflammation4 | | | | | | |
| 6 month_{—} | 0 | 0.305893 | 0.135197 | 0.442257 | 0 | 0.043837 |
| Inflammation1 | | | | | | |
| 6 month_{—} | 0.080726 | 0.017153 | 0.012021 | 0.212079 | 0.008773 | 0.533524 |
| Inflammation2 | | | | | | |
| 6 month_{—} | 0.030113 | 0.038095 | 0.018742 | 0.85738 | 0 | 0 |
| Inflammation3 | | | | | | |
| 1 year_{—} | 0 | 0.414023 | 0.163068 | 0.283116 | 0 | 0.121129 |
| Inflammation1 | | | | | | |
| 1 year_{—} | 0 | 0.197477 | 0.388474 | 0.17059 | 0 | 0.214422 |
| Inflammation2 | | | | | | |
| 1 year_{—} | 0 | 0.480573 | 0.023508 | 0.446793 | 0.017402 | 0.263571 |
| Inflammation3 | | | | | | |
| 1 year_{—} | 0.025621 | 0.154508 | 0.061471 | 0.311214 | 0 | 0.445662 |
| Inflammation4 | | | | | | |

| | T cells CD4 | T cells | T cells | T cells | NK cells |
|---|---|---|---|---|---|
| Mixture | memory | follicular | regulatory | gamma delta | resting |
| 0 month_{—} | 0 | 0 | 0.12641 | 0.071654 | 0 |
| Inflammation1 | | | | | |
| 0 month_{—} | 0.078301 | 0 | 0 | 0.058875 | 0.167308 |
| Inflammation2 | | | | | |
| 0 month_{—} | 0 | 0.064749 | 0.159431 | 0 | 0 |
| Inflammation3 | | | | | |
| 0 month_{—} | 0 | 0.199692 | 0.211717 | 0 | 0 |
| Inflammation4 | | | | | |
| 4 month_{—} | 0 | 0.000549 | 0.261345 | 0 | 0.433064 |
| Inflammation1 | | | | | |
| 4 month_{—} | 0.074232 | 0 | 0 | 0 | 0.38762 |
| Inflammation2 | | | | | |
| 4 month_{—} | 0.091241 | 0.126879 | 0.200348 | 0 | 0.092597 |
| Inflammation3 | | | | | |
| 4 month_{—} | 0.009517 | 0 | 0.098911 | 0 | 0.073847 |
| Inflammation4 | | | | | |
| 6 month_{—} | 0 | 0.210735 | 0.340157 | 0 | 0 |
| Inflammation4 | | | | | |
| 6 month_{—} | 0.138999 | 0.045803 | 0 | 0.248472 | 0 |
| Inflammation1 | | | | | |
| 6 month_{—} | 0.183971 | 0.030929 | 0 | 0 | 0.357882 |
| Inflammation2 | | | | | |
| 6 month_{—} | 0 | 0.201939 | 0.560859 | 0 | 0.047486 |
| Inflammation3 | | | | | |
| 1 year_{—} | 0 | 0.063301 | 0.391007 | 0.011551 | 0 |
| Inflammation1 | | | | | |
| 1 year_{—} | 0 | 0 | 0.287218 | 0 | 0 |
| Inflammation2 | | | | | |
| 1 year_{—} | 0 | 0.021318 | 0 | 0 | 0 |
| Inflammation3 | | | | | |
| 1 year_{—} | 0.001212 | 0.081481 | 0.312267 | 0 | 0 |
| Inflammation4 | | | | | |

| | | | | | | Dendritic |
|---|---|---|---|---|---|---|
| | NK cells | | Macrophage | Macrophages | Macrophages | cells |
| Mixture | activated | Monocytes | M0 | M1 | M2 | resting |
| 0 month_{—} | 0.264951 | 0.033701 | 0 | 0.038722 | 0.032796 | 0.041548 |
| Inflammation1 | | | | | | |
| 0 month_{—} | 0 | 0 | 0.013412 | 0.095119 | 0.128169 | 0 |
| Inflammation2 | | | | | | |
| 0 month_{—} | 0.195629 | 0.060695 | 0.068587 | 0.120597 | 0.070413 | 0.083357 |
| Inflammation3 | | | | | | |
| 0 month_{—} | 0.170421 | 0.056678 | 0 | 0.038259 | 0.063815 | 0 |
| Inflammation4 | | | | | | |
| 4 month_{—} | 0 | 0.095939 | 0 | 0.087122 | 0.046958 | 0.154949 |
| Inflammation1 | | | | | | |
| 4 month_{—} | 0 | 0.043942 | 0 | 0.019025 | 0.114327 | 0.02145 |
| Inflammation2 | | | | | | |
| 4 month_{—} | 0.009378 | 0.05174 | 0 | 0.022247 | 0.109007 | 0.06521 |
| Inflammation3 | | | | | | |
| 4 month_{—} | 0.018155 | 0 | 0.083649 | 0.07471 | 0.050803 | 0.047286 |
| Inflammation4 | | | | | | |
| 6 month_{—} | 0.391134 | 0 | 0.625977 | 0.700996 | 0.208371 | 0 |
| Inflammation4 | | | | | | |
| 6 month_{—} | 0 | 0 | 0 | 0.112019 | 0.184214 | 0.015149 |
| Inflammation1 | | | | | | |
| 6 month_{—} | 0 | 0 | 0.028833 | 0.115322 | 0.095857 | 0.064012 |
| Inflammation2 | | | | | | |
| 6 month_{—} | 0.19827 | 0.025355 | 0.056295 | 0.16807 | 0.113836 | 0.141757 |
| Inflammation3 | | | | | | |
| 1 year_{—} | 0.269998 | 0.041634 | 0 | 0.080913 | 0.295775 | 0.08847 |
| Inflammation1 | | | | | | |
| 1 year_{—} | 0.252342 | 0.080014 | 0 | 0.039145 | 0.141608 | 0.049931 |
| Inflammation2 | | | | | | |
| 1 year_{—} | 0.212349 | 0.058105 | 0 | 0.024881 | 0.050113 | 0.04673 |
| Inflammation3 | | | | | | |
| 1 year_{—} | 0.135224 | 0.043035 | 0.006191 | 0.022475 | 0.079242 | 0.010844 |
| Inflammation4 | | | | | | |

| | | Mast | | | |
|---|---|---|---|---|---|
| | Dendritic | cells | Mast cells | | |
| Mixture | cells | resting | activated | Eosinophils | Neutrophils |
| 0 month_{—} | 0.048034 | 0.59584 | 0 | 0 | 0 |
| Inflammation1 | | | | | |
| 0 month_{—} | 0.050474 | 0.740656 | 0 | 0 | 0.053728 |
| Inflammation2 | | | | | |
| 0 month_{—} | 0.004695 | 0.345754 | 0.003337 | 0 | 0 |
| Inflammation3 | | | | | |
| 0 month_{—} | 0 | 0 | 0.705699 | 0.085736 | 0 |
| Inflammation4 | | | | | |
| 4 month_{—} | 0 | 0.079234 | 0.359588 | 0.077492 | 0 |
| Inflammation1 | | | | | |
| 4 month_{—} | 0 | 0.048306 | 0.074425 | 0.065608 | 0.014643 |
| Inflammation2 | | | | | |
| 4 month_{—} | 0 | 0.457802 | 0.069522 | 0 | 0 |
| Inflammation3 | | | | | |
| 4 month_{—} | 0 | 0.812892 | 0.024123 | 0 | 0.010993 |
| Inflammation4 | | | | | |
| 6 month_{—} | 0 | 0.591176 | 0 | 0 | 0 |
| Inflammation4 | | | | | |
| 6 month_{—} | 0 | 0.096952 | 0 | 0.280546 | 0 |
| Inflammation1 | | | | | |
| 6 month_{—} | 0.003848 | 0.108293 | 0.143612 | 0.035435 | 0.01097 |
| Inflammation2 | | | | | |
| 6 month_{—} | 0 | 0.09185 | 0.027909 | 0 | 0.017112 |
| Inflammation3 | | | | | |
| 1 year_{—} | 0 | 0.154001 | 0 | 0 | 0.043048 |
| Inflammation1 | | | | | |
| 1 year_{—} | 0 | 0.49925 | 0 | 0 | 0.044381 |
| Inflammation2 | | | | | |
| 1 year_{—} | 0.05906 | 0.05805 | 0.059538 | 0.045046 | 0.0635 |
| Inflammation3 | | | | | |
| 1 year_{—} | 0.021964 | 0.313461 | 0.052928 | 0.006709 | 0.014606 |
| Inflammation4 | | | | | |

### Example 4: Confirmation of scRNA Analysis Characteristics of Paired EMPD Samples

### Example 4-1: Results of Single-Cell RNA Transcriptomic Analysis of Primary Extramammary Paget Disease (EMPD)

The results of single-cell RNA transcriptomic analysis of primary extramammary Paget disease (EMPD) are as follows.

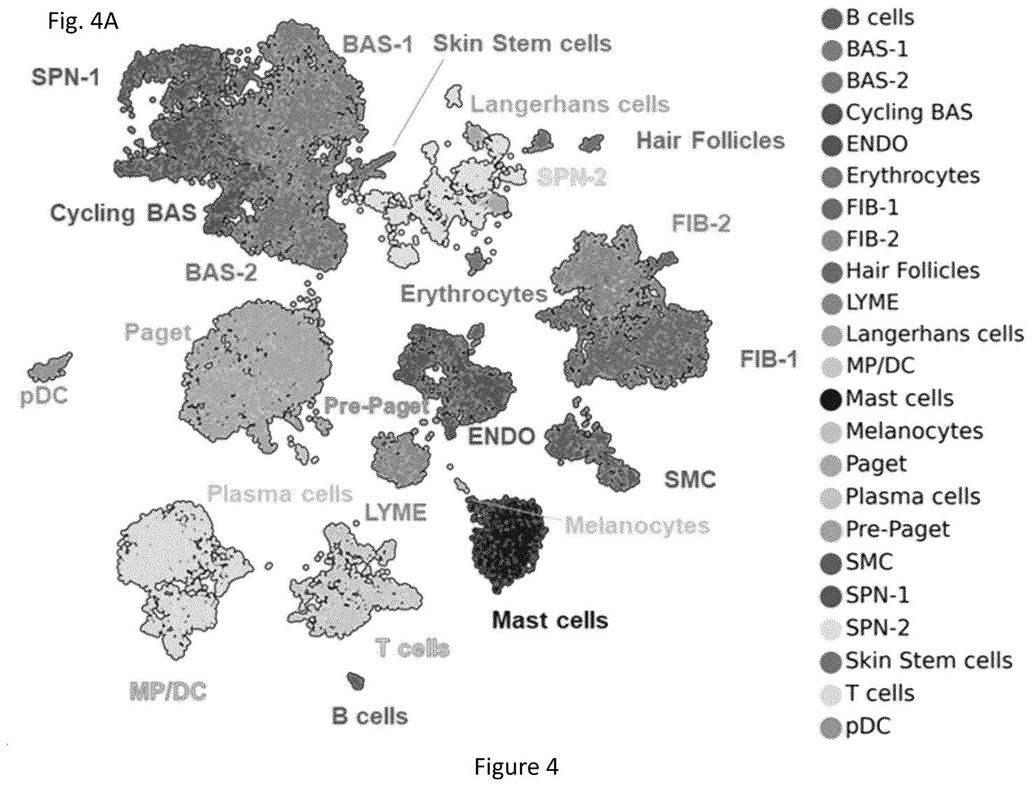

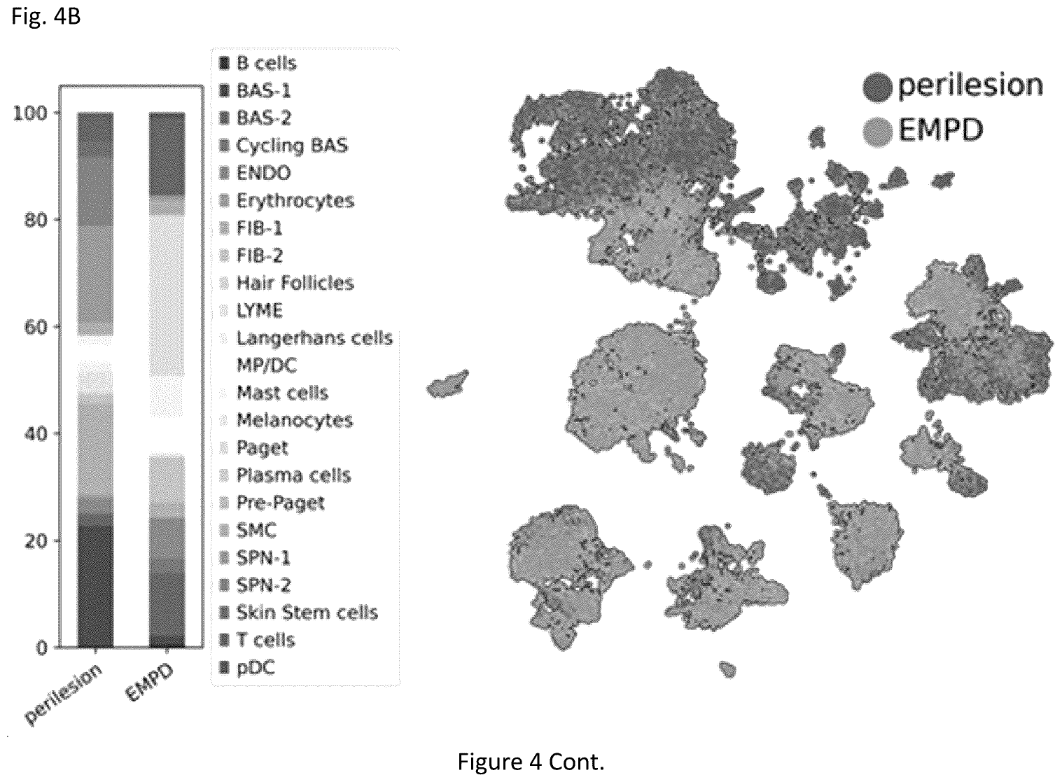

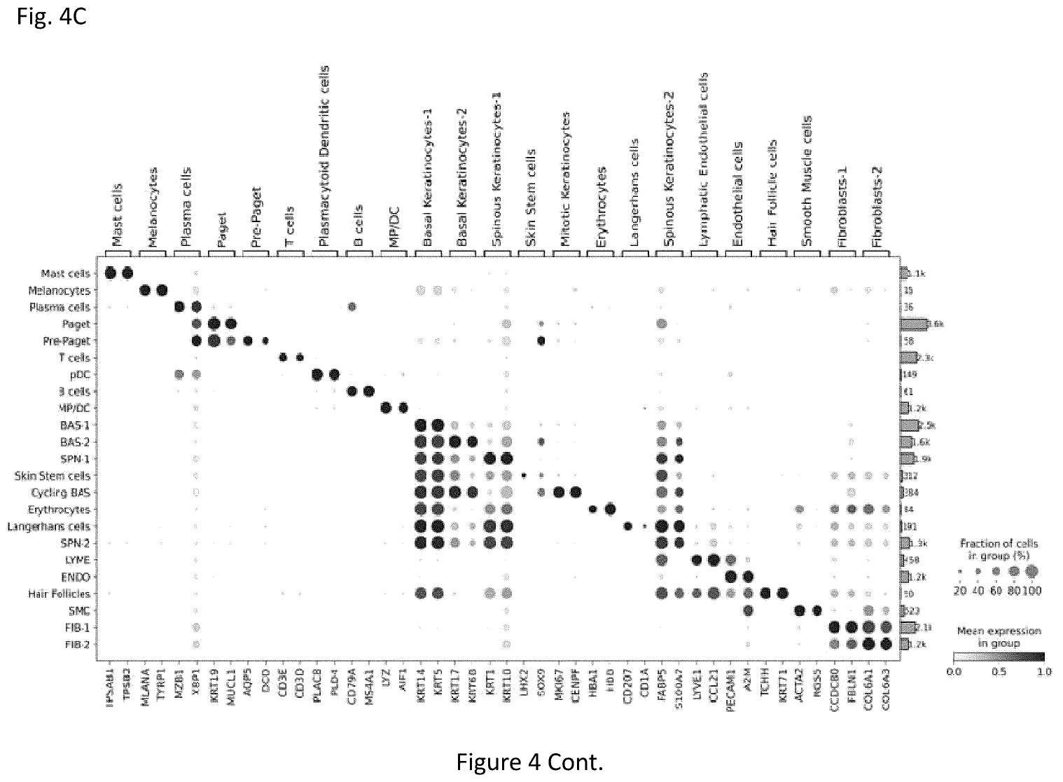

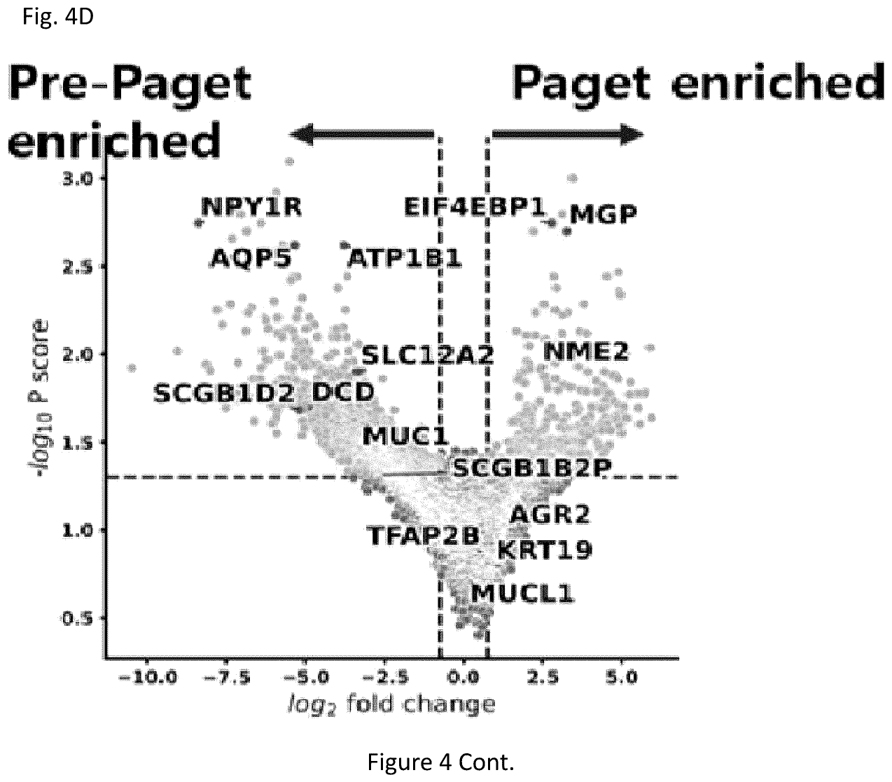

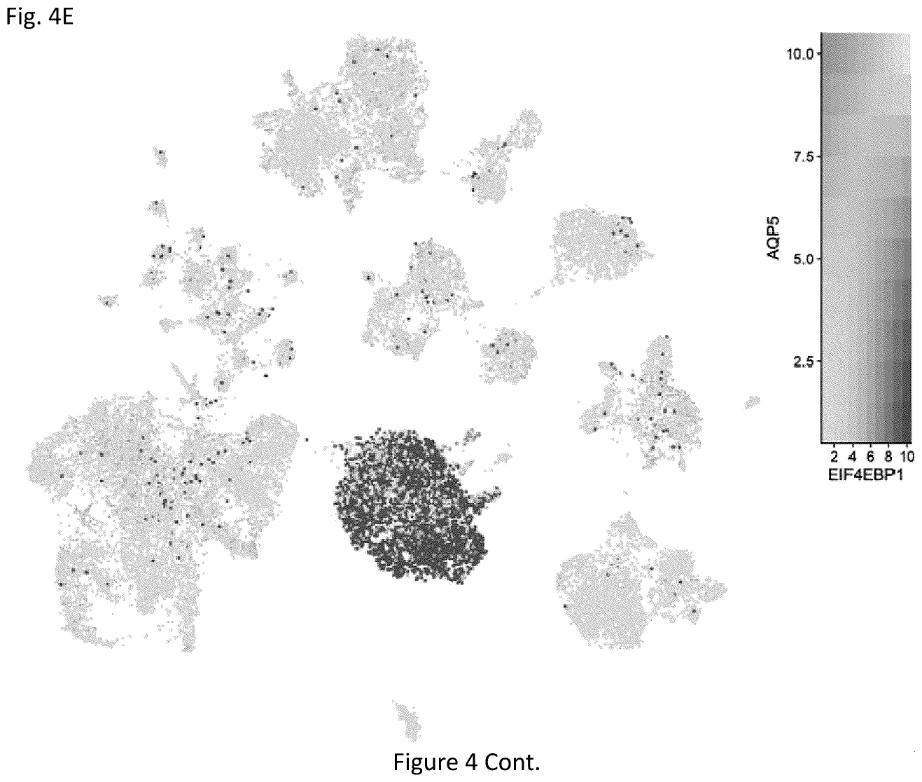

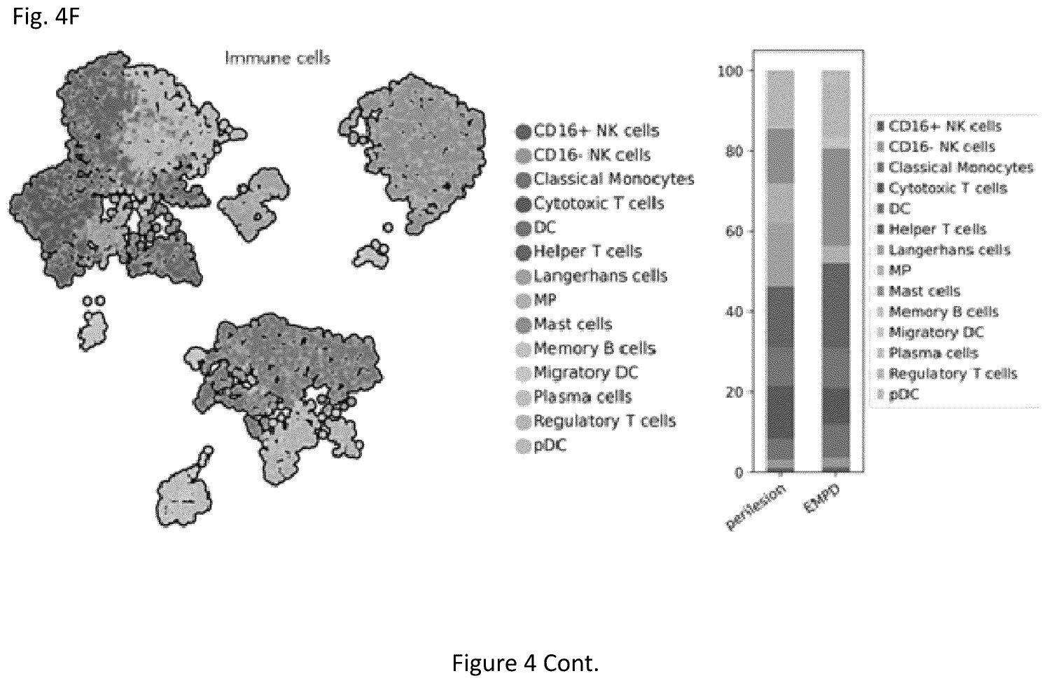

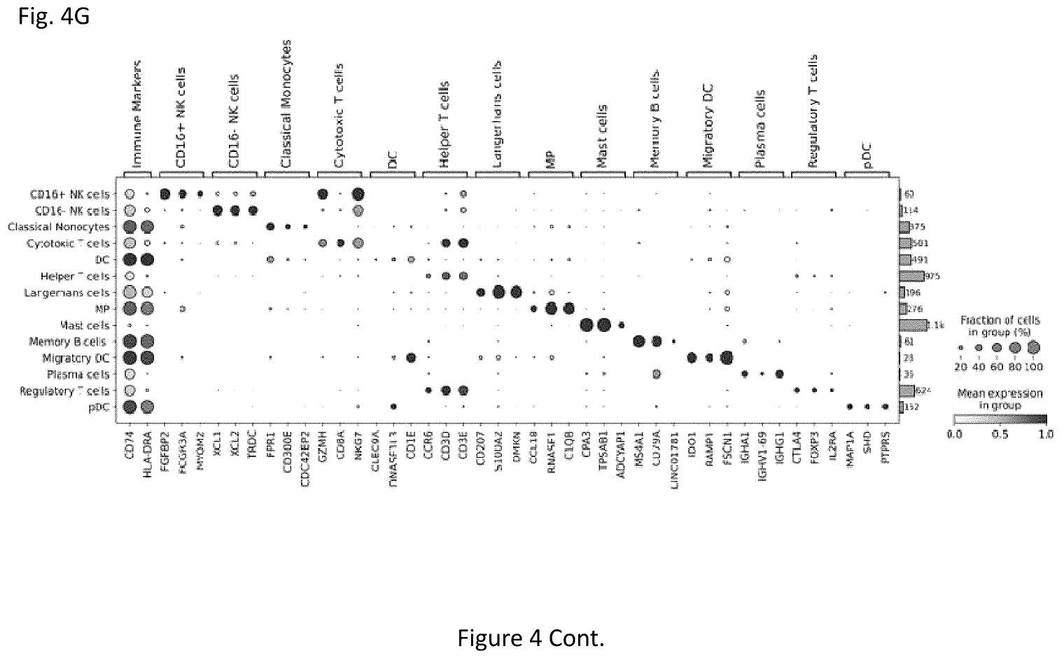

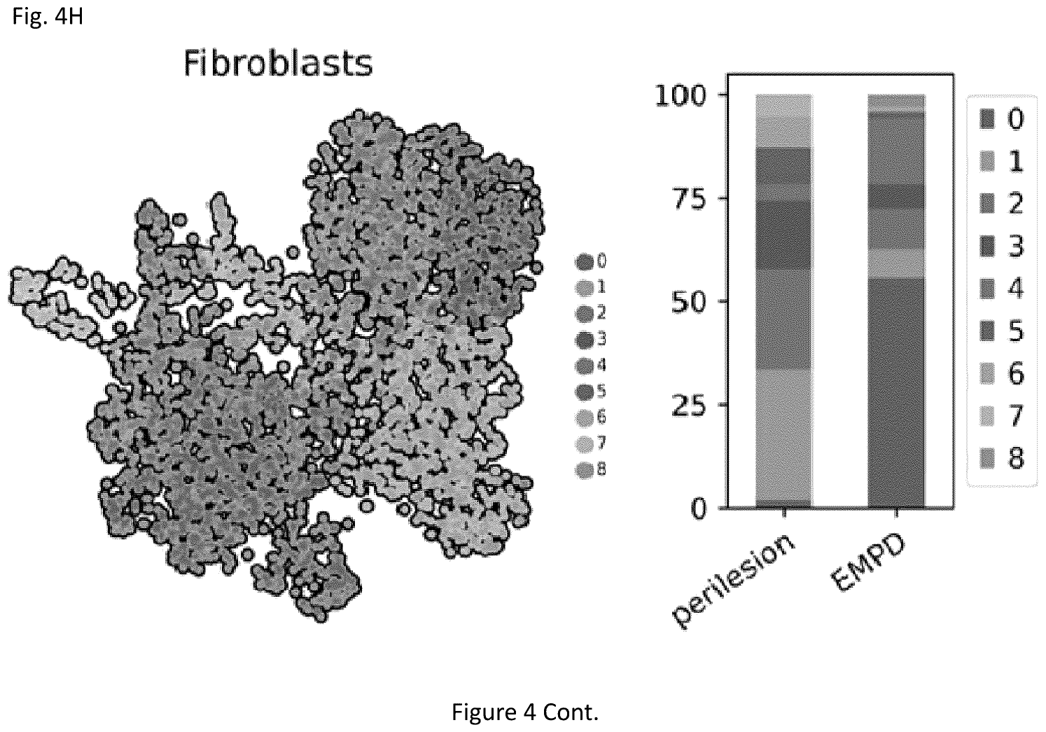

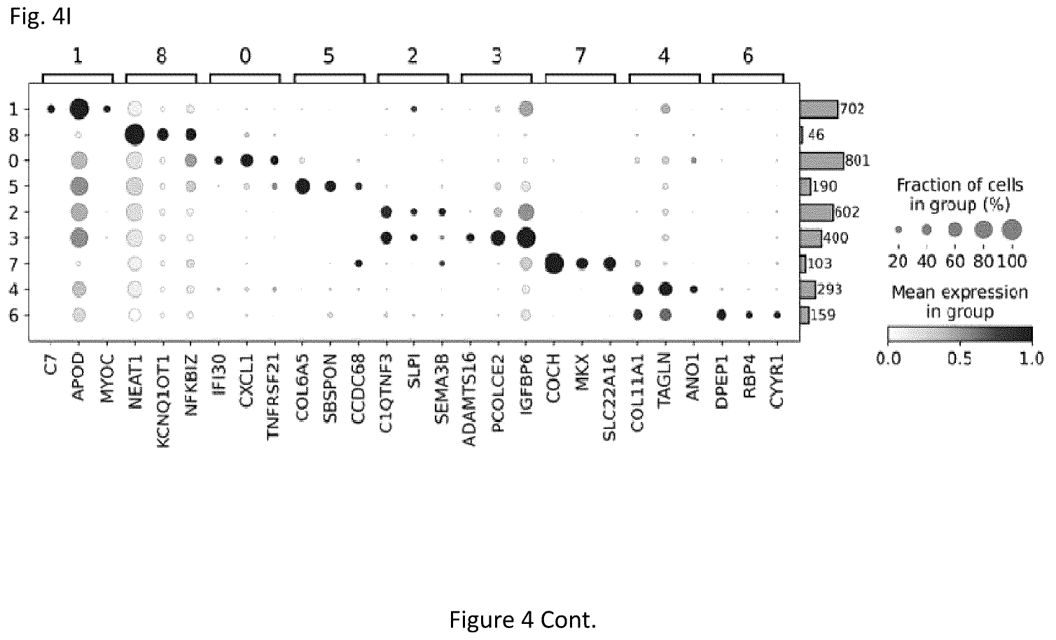

First, UMAP visualization analysis of EMPD cell types and perilesional skin samples is shown in FIG. 4A. Also, as shown in FIG. 4B, it was confirmed that 3,637 Paget cells accounted for approximately 30% of the total cells in the EMPD samples. Each sample contained 12,185 EMPD cells and 10,255 perilesional cells.

Also, according to the dot plot of genetic markers in FIG. 4C, each cell type was defined by standard skin genetic markers. According to the volcano plot of differential gene expression between Paget cells and pre-Paget cells in FIG. 4D, it was confirmed that the pre-Paget cells had unique expression of genes such as AQP5 and DCD, indicating unique properties compared to the Paget cells. In particular, AGR2 was confirmed to be a gene that appeared not only in the Paget cells but also in the pre-Paget cells. Meanwhile, according to the UMAP in FIG. 4E, it was confirmed that the co-expression pattern of AQP5 (Pre-Paget marker) and EIF4EBP1 (Paget marker) was distinct.

Additionally, to confirm the relationship between SPDEF and pre-Paget cells, the top expressed genes in pre-Paget cells were examined.

As a result, SPDEF had scores of 5.851562, p-value of 4.87E-09, pvals_adj of 2.09E-06, and logfoldchanges of 3.04298, confirming that it is a significant marker in pre-Paget cells.

FIGS. 4F and 4G show the cell types of immune subpopulations and genetic markers of immune subpopulation cell types, respectively. Also, FIGS. 4H and 4I show the subclusters for fibroblasts and genetic markers for fibroblast subtypes. According to FIG. 4H, the EMPD and perilesional samples were confirmed to have a heterogeneous cell ratio between subpopulations. Also, according to FIG. 4I, it was confirmed that differences in genetic markers and cell type ratios for fibroblast subtypes was indicative of cancer-related fibroblast characteristics.

### Example 4-2: Inference of Chromosome Copy Number Variations

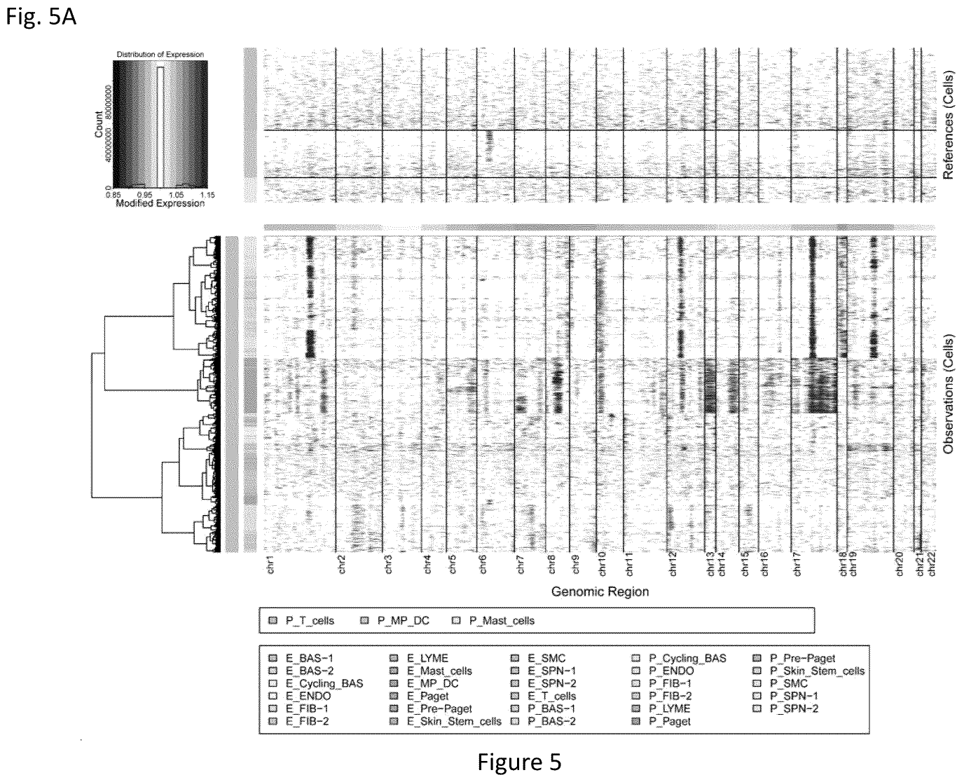

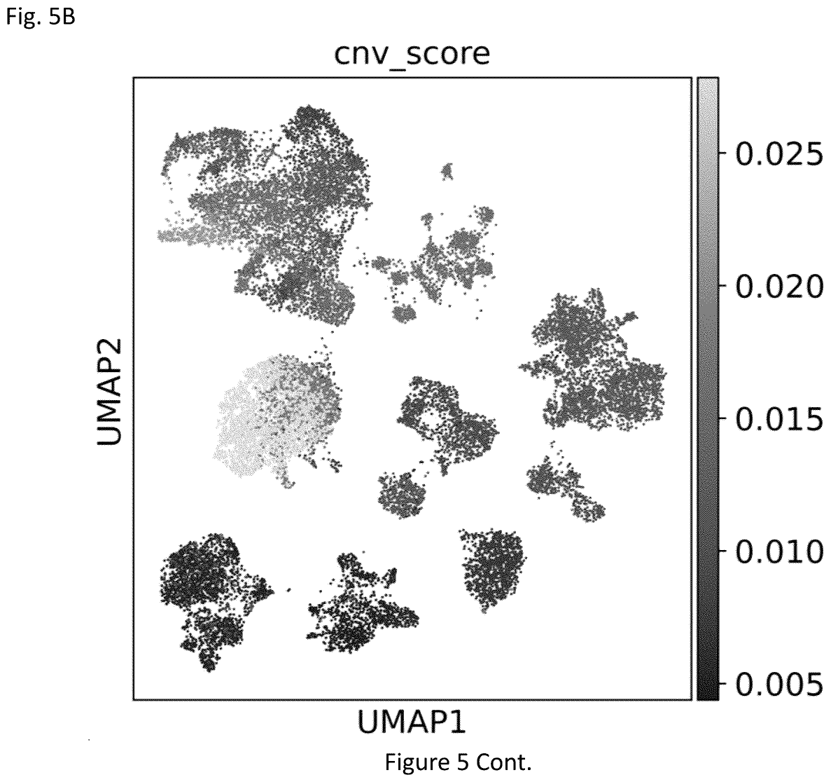

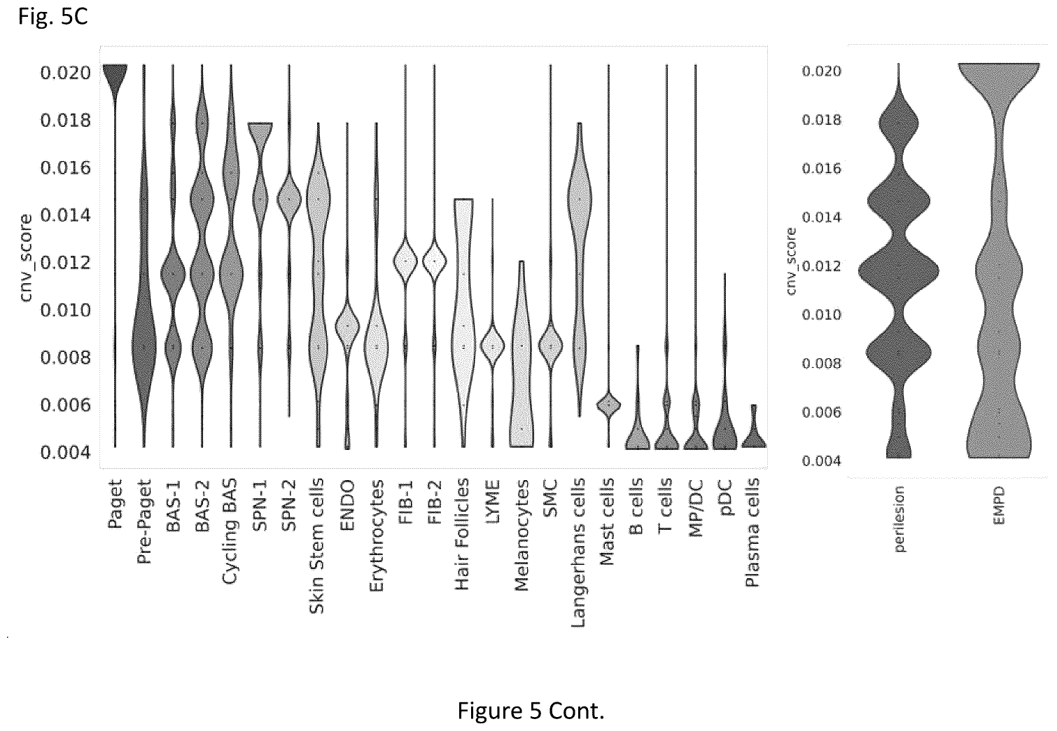

First, the InferCNV heatmap of cell types divided by sample source is shown in FIG. 5A. T cells, dendritic cells, and mast cells from perilesional samples were used as standards, and Paget cells were found to show a large increase in chromosome 17.

FIG. 5B shows the copy number variations displayed in UMAP.

Also, FIG. 5C shows the copy number variation scores displayed as violin plots by cell type and sample source. Here, it was confirmed that the Paget cells had a relatively higher copy number variation scores compared to other cell types.

### Example 4-3: Cell Trajectory Analysis of EMPD Epithelial Cells

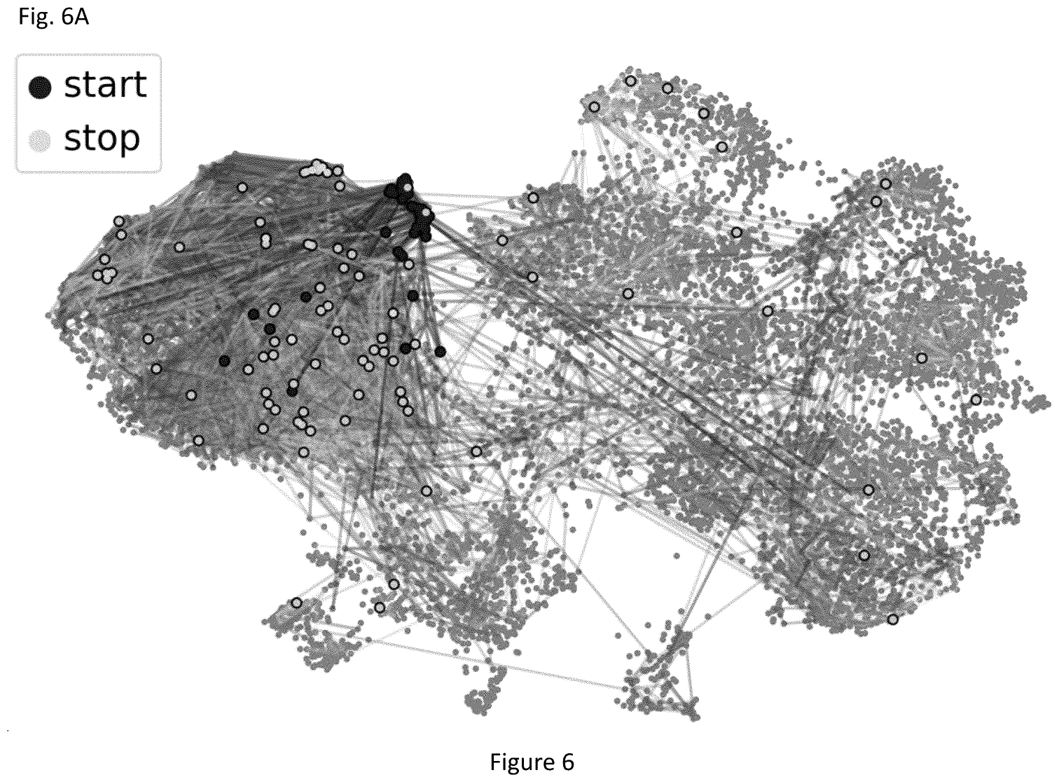

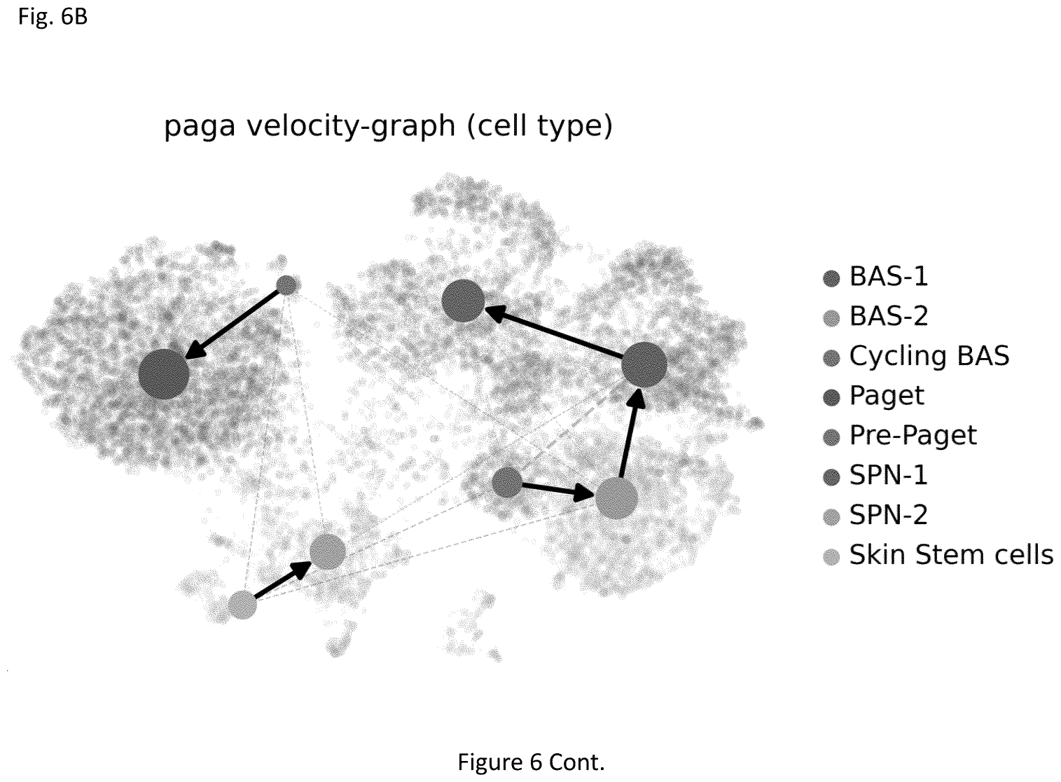

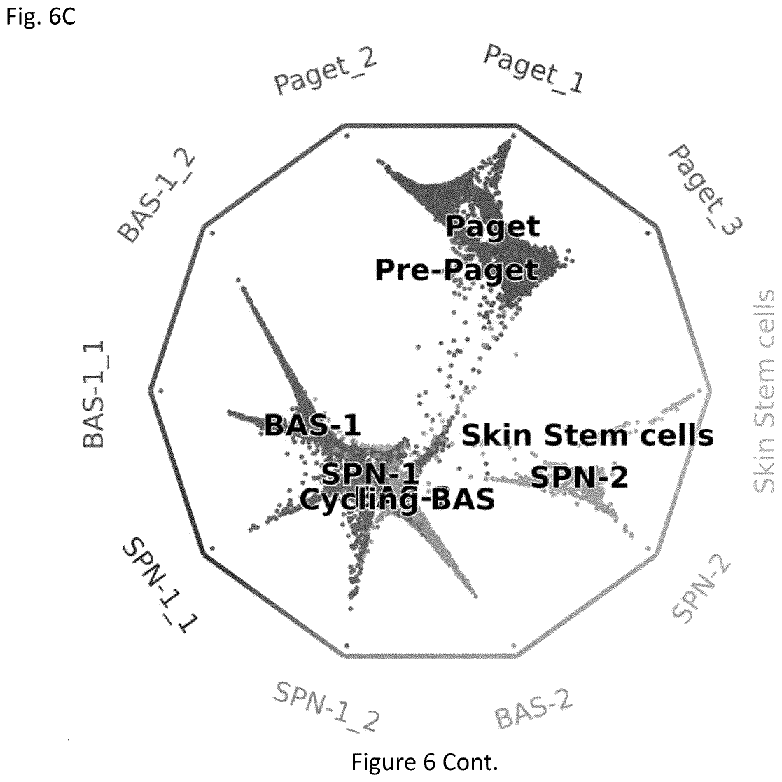

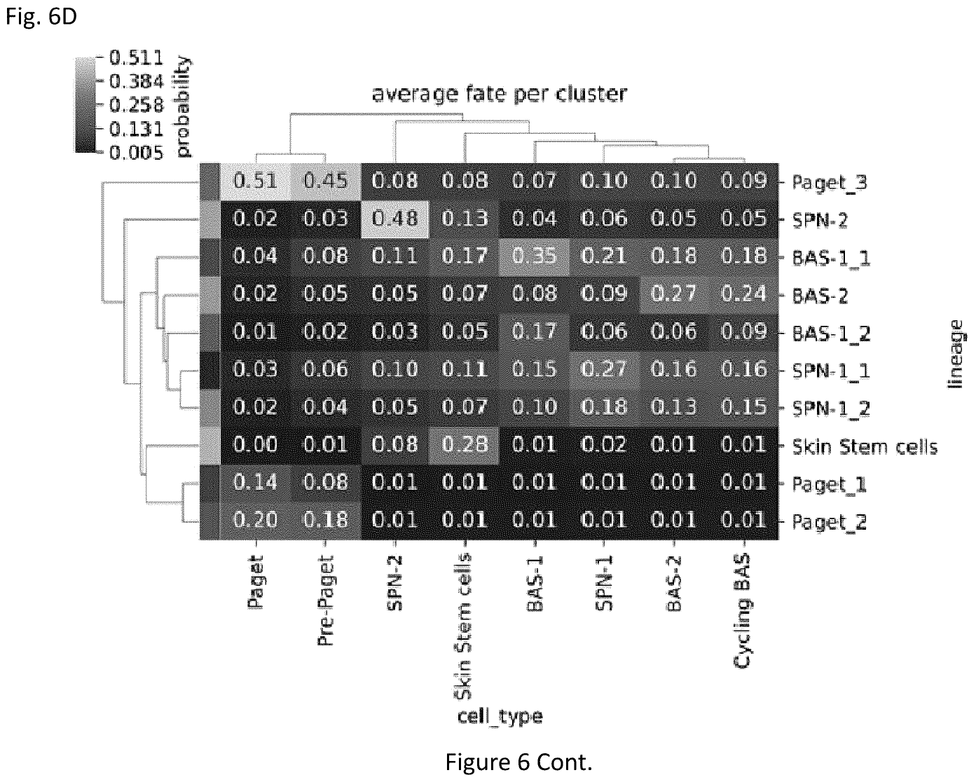

FIG. 6A shows a random walk plot, confirming that cell maturation ended in Paget cells when pre-Paget cell seeds are provided. Also, FIG. 6B shows the UMAP of a PAGA speed graph. Here, it was confirmed that the basal keratinocytes in the epithelial layer normally matured into spinous keratinocytes.

In a similar context, it was examined whether the pre-Paget cells are connected to Paget cells. FIG. 6C shows a circular projection plot according to the fate probability for pre-Paget cells leading to Paget cells.

FIG. 6D shows a heatmap of fate probabilities. Here, each cell type displayed in the lineage represents a different final state within each cluster.

### Example 4-4: Top 20 Lineage Driver Genes in Paget Cells

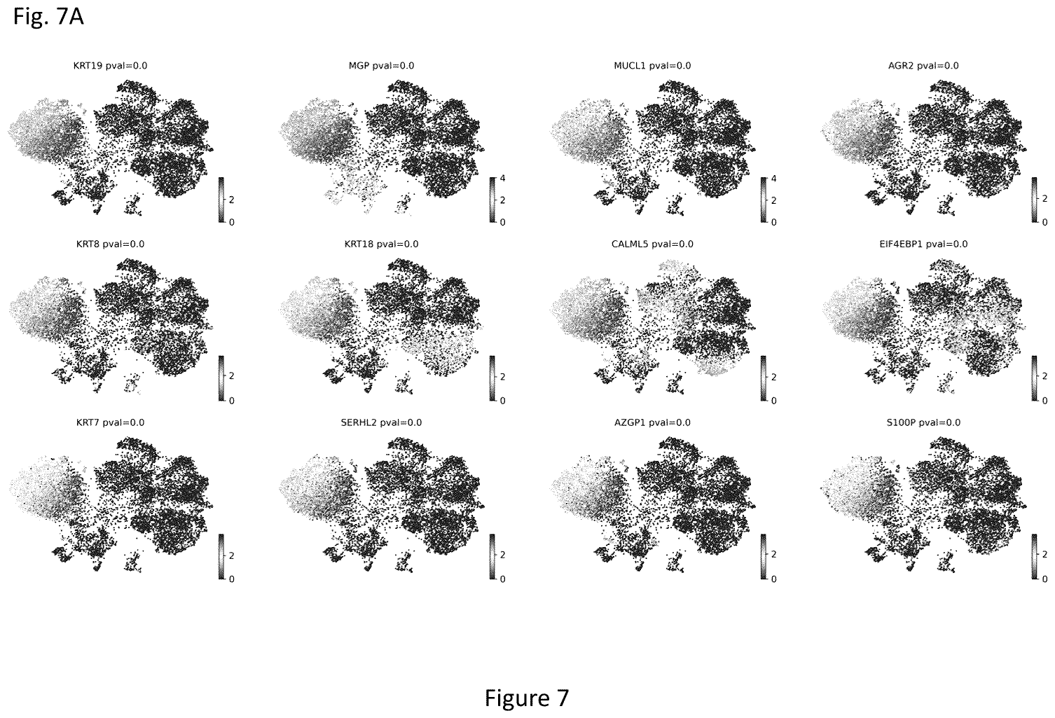

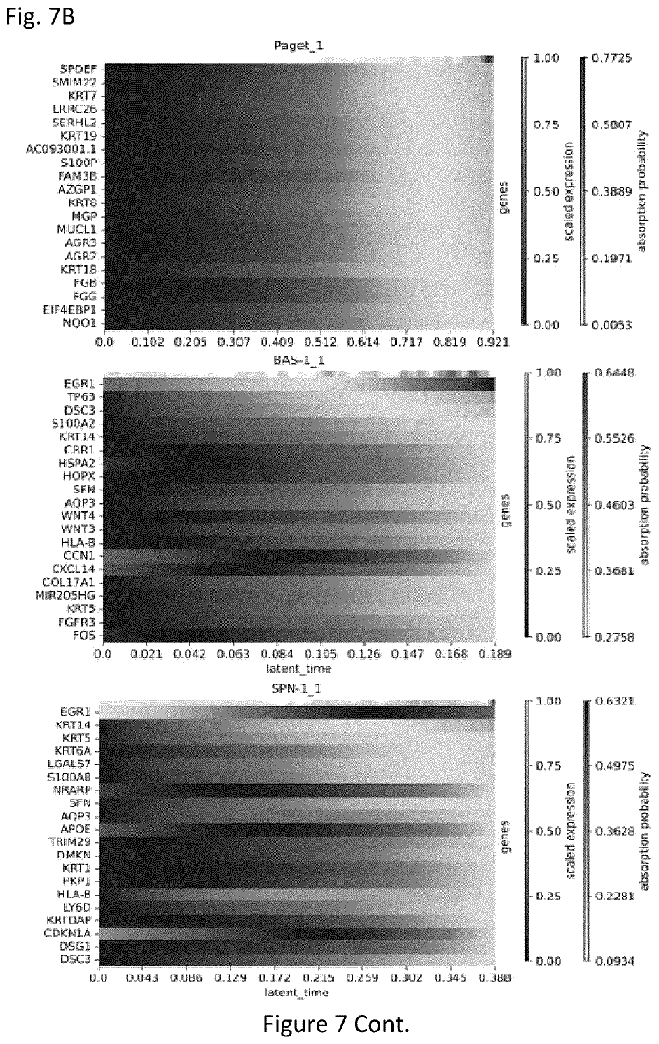

According to FIG. 7A, the top 12 lineage driving genes for Paget cells were identified. In particular, AGR2 was discovered as a new Paget marker as it was found to be expressed in Paget cells and pre-Paget cells at similar or higher levels than other markers.

Also, FIG. 7B shows lineage driver gene expression according to latent time. Based on these results, the genes were sequentially expressed in basal cells and spiny cells according to cell maturation. Although the relatively early latent phase in Paget cells has not been elucidated, distinct driver genes were observed in the Paget cells.

That is, it was confirmed that AGR2 is a novel gene highly expressed in Paget cells, and not only was it identified along with previously known genes, but AGR2 also was verified to exhibit the characteristics of EMPD in that it is a gene associated with EMT.

### Example 4-5: Cell-to Cell Communication Analysis

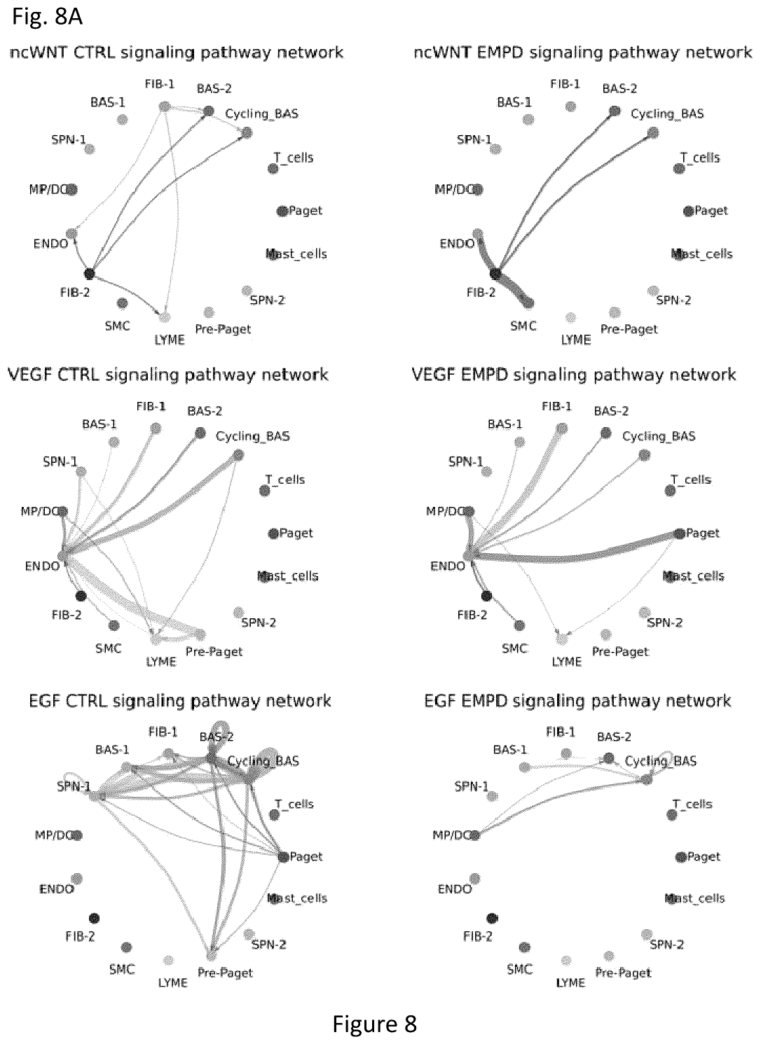

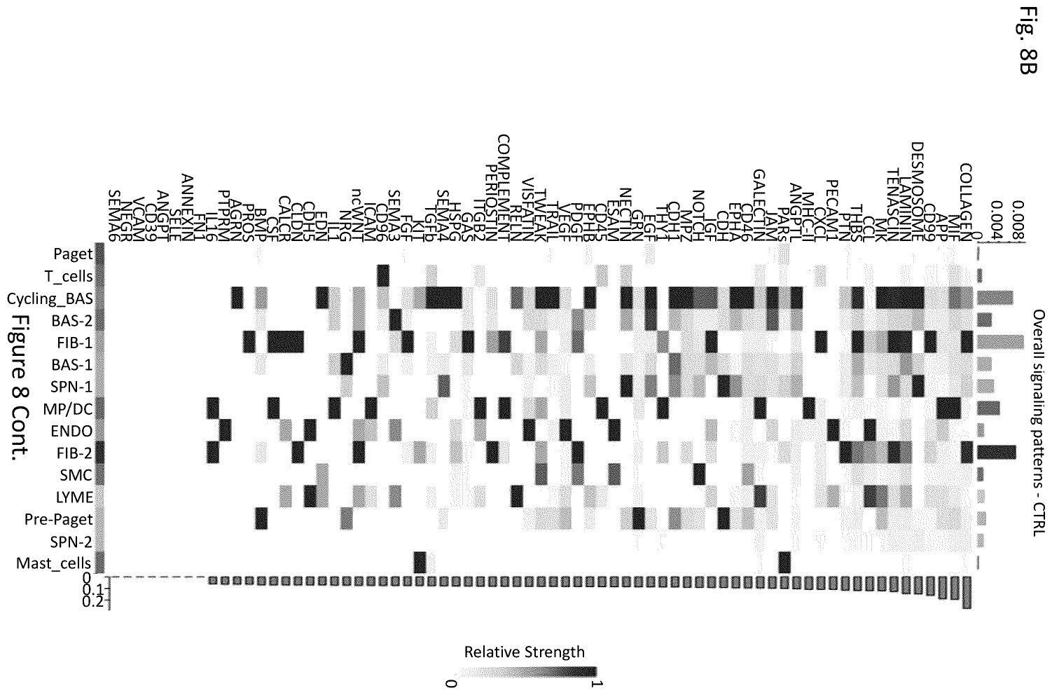

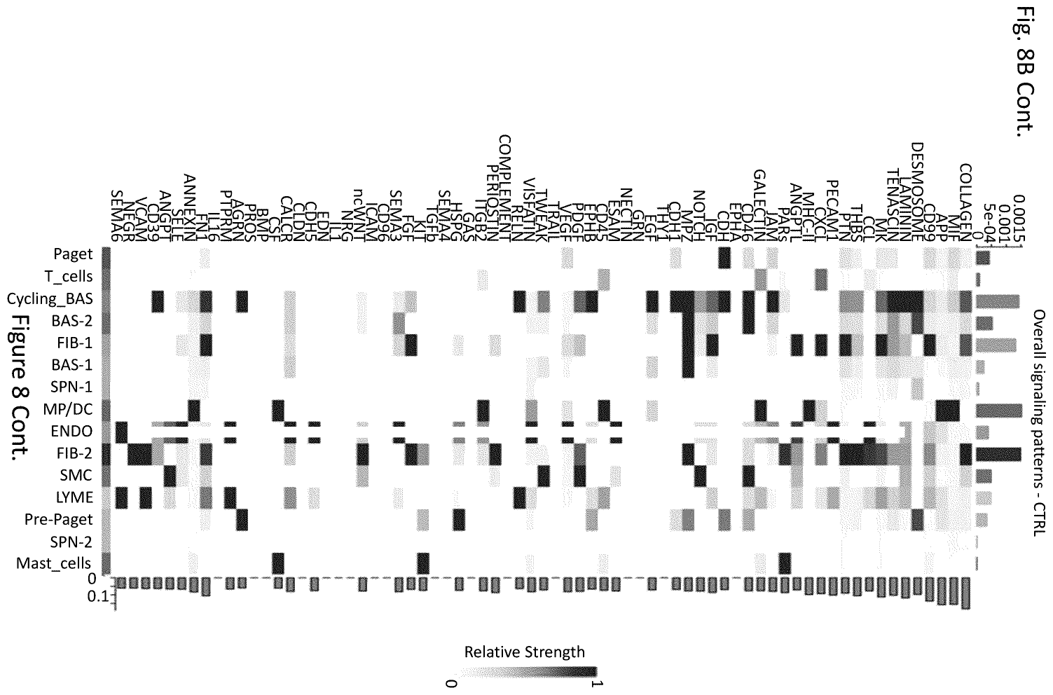

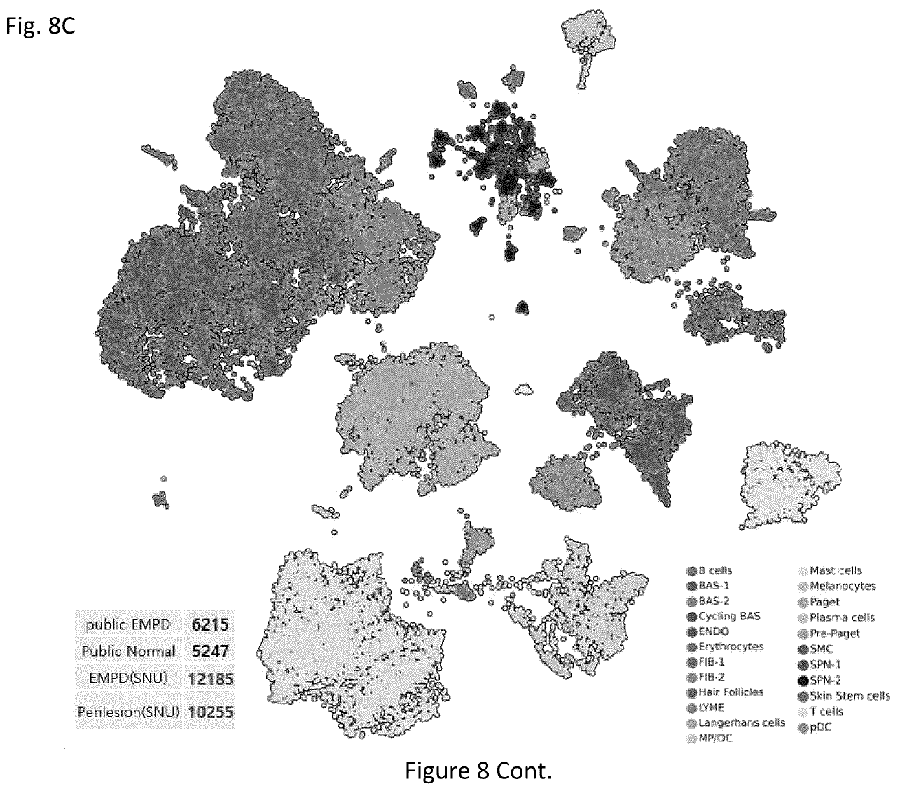

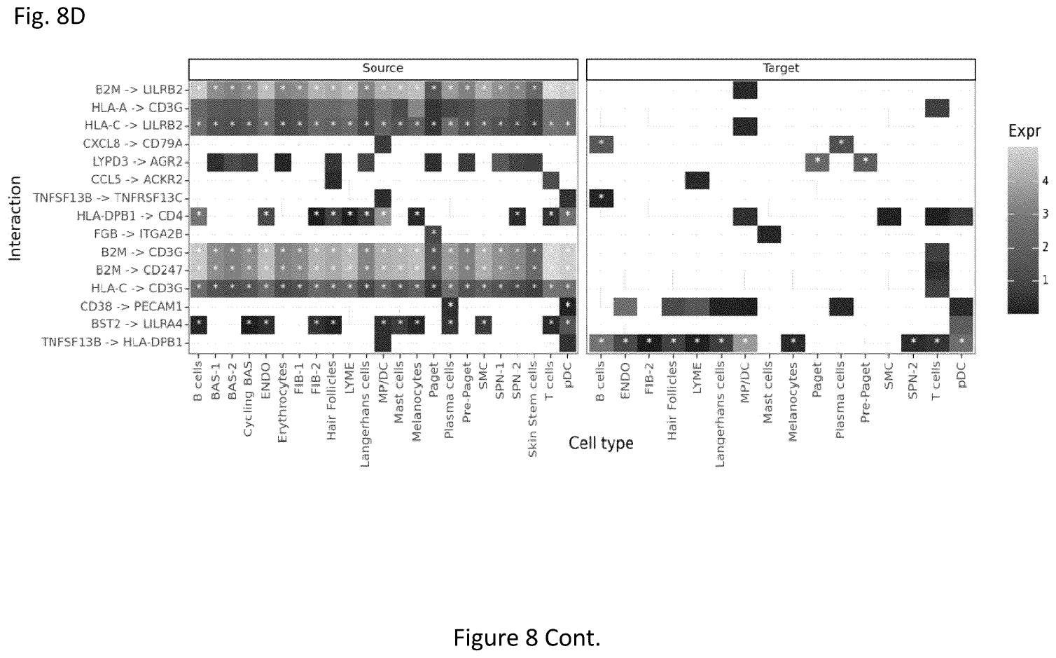

FIG. 8A shows important differential signaling pathways between sample conditions. Here, non-canonical WNT signals, VEGF signals, and EGF signals are indicated. Specifically, Paget cells affecting endothelial cells have been described in the VEGF signaling pathway.

FIG. 8B shows the overall signal patterns regarding the state. It was confirmed that most signaling patterns were concentrated in fibroblasts at risk, and EMPD had a higher macrophage/dendritic cell interaction intensity than at risk.

FIG. 8C shows integration2 with public EMPD data. Here, the cell types were annotated with cell type names using the Celltypist modeling function.

FIG. 8D shows the analysis of the top 15 influential receptor-ligands of the integrated EMPD atlas for EMPD cell types. Based on these results, AGR2 ligand signaling appears to be a specific interaction between Paget cells and pre-Paget cells.

### Example 4-6: Integration of Public DBs and Prediction of Potential Drugs by Signaling Pathways

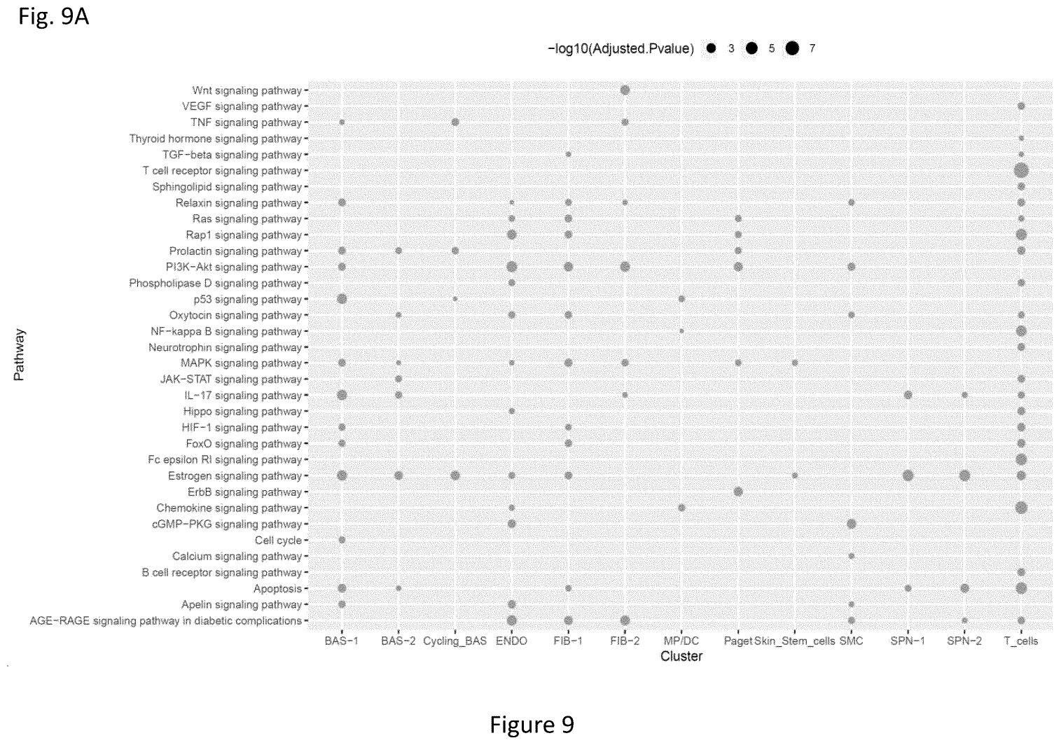

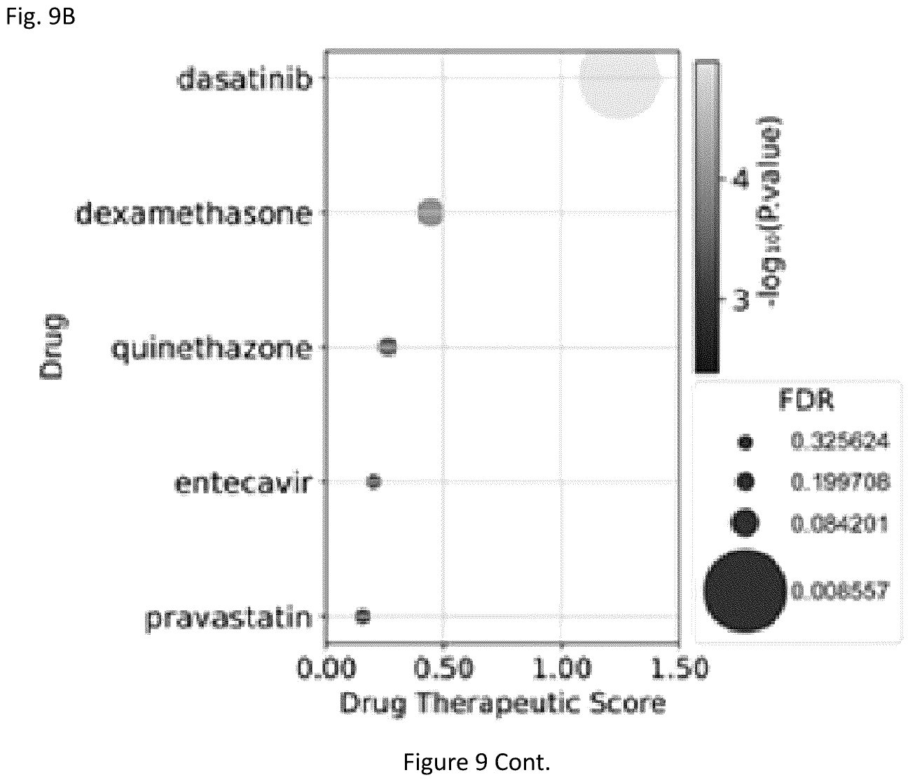

FIG. 9A shows canonical pathways within cell types. Here, ErbB signaling was labeled as a Paget-specific pathway.

FIG. 9B shows the prediction of potential drug efficacy. Here, the gene expression in pre-Paget cells was compared with that of the related Paget cells. Dasatinib, a known ErbB2 (HER-2)/Src inhibitor, showed the highest therapeutic score.

Through sc-RNA analysis, a novel genetic marker AGR2 was identified, which was consistently observed in DEG analysis, trajectory driver gene analysis, and cell-to-cell communication analysis.

The description of the present invention described above is for illustrative purposes, and it should be understood that those of ordinary skill in the art to which the present invention pertains can easily modify embodiments into other specific forms without changing the technical idea or essential features described in this specification. Therefore, it should be understood that all the embodiments described above are illustrative in all respects and not restrictive.

What is claimed is:

1. A method of preventing or treating extramammary Paget disease, comprising the following steps:
measuring the level of one or more selected from the group consisting of an SAM pointed domain containing ETS transcription factor (SPDEF), arginase 2 (ARG2), and adipocyte enhancer-binding protein 1 (ABEP1) from a biological sample isolated from a subject;
comparing the measured level of one or more selected from the group consisting of SPDEF, ARG2, and ABEP1 with the level in a biological sample isolated from the control;
when the level of any one or more selected from the group consisting of SPDEF, ARG2, and ABEP1 in the biological sample isolated from the subject is higher than the level in the biological sample isolated from the control, determining this case to be extramammary Paget disease; and
administering an agent for preventing or treating extramammary Paget disease to a subject determined to have extramammary Paget disease.

2. The method of claim 1, wherein the extramammary Paget's disease is non-invasive extramammary Paget's disease, or invasive extramammary Paget's disease.

3. The method of claim 2, wherein the non-invasive extramammary Paget's disease is early extramammary Paget's disease, or non-early extramammary Paget's disease.

4. The method of claim 1, further comprising:
when the level of ARG2 in the biological sample isolated from the subject is higher than the level in the biological sample isolated from the control, determining this case to be non-invasive extramammary Paget disease; or
when the level of ABEP1 in the biological sample isolated from the subject is higher than the level in the biological sample isolated from the control, determining this case to be invasive extramammary Paget disease,
administering an agent for preventing or treating non-invasive extramammary Paget disease, or invasive extramammary Paget disease to a subject determined to have non-invasive extramammary Paget disease, or invasive extramammary Paget disease respectively.

5. The method of claim 1, further comprising:
When the biological sample isolated from the subject is a pre-Paget cell,
when the level of any one or more selected from the group consisting of SPDEF and AGR2 in the biological sample isolated from the subject is higher than the level in the biological sample isolated from the control, determining this case to early extramammary Paget's disease;
administering an agent for preventing or treating early extramammary Paget disease to a subject determined to have early extramammary Paget disease.

6. The method of claim 1, wherein the biological sample is any one selected from the group consisting of tissue, blood, serum, whole blood, plasma, urine, saliva, cells, organs, bone marrow, a fine needle aspiration specimen, a core needle biopsy specimen, and a vacuum-assisted suction biopsy specimen.

7. The method of claim 2, wherein the invasive extramammary Paget disease drug is extramammary Paget disease that persists despite the administration of an extramammary Paget disease drug.

8. A method of screening for an agent for preventing or treating extramammary Paget disease, comprising the following steps:
measuring the level of any one or more selected from the group consisting of an SAM pointed domain containing ETS transcription factor (SPDEF), arginase 2 (ARG2), and adipocyte enhancer-binding protein 1 (ABEP1) from a biological sample isolated from an animal model of extramammary Paget disease administered a candidate material; and
when the level of any one or more selected from the group consisting of SPDEF, ARG2, and AEBP1 in the biological sample isolated from the animal model decreases, selecting the candidate material as an agent for preventing or treating extramammary Paget disease.

9. The method of claim 8, further comprising:
when the level of AGR2 in the biological sample isolated from the animal model decreases, selecting the candidate material as an agent for preventing or treating non-invasive extramammary Paget disease; or
when the level of ABEP1 in the biological sample isolated from the animal model decreases, selecting the candidate material as an agent for preventing or treating invasive extramammary Paget disease.

10. A kit for screening for an agent for preventing or treating extramammary Paget disease, comprising:
i) a composition comprising, as an active ingredient, an agent for measuring the level of one or more selected from the group consisting of an SAM pointed domain containing ETS transcription factor (SPDEF), arginase 2 (ARG2), and adipocyte enhancer-binding protein 1 (ABEP1); and
ii) instructions.
